(19) United States
(12) Patent Application Publication
(10) Pub. No.: US 2012/0040977 A1
(43) Pub. Date: Feb. 16, 2012

(54) INHIBITORS OF HCV NS5A

(75) Inventors: Leping Li, Burlingame, CA (US); Min Zhong, Palo Alto, CA (US)

(73) Assignee: PRESIDIO PHARMACEUTICALS, INC., San Francisco, CA (US)

(21) Appl. No.: 13/202,726

(22) PCT Filed: Feb. 22, 2010
(86) PCT No.: PCT/US10/24946

§ 371 Date: Nov. 1, 2011

Related U.S. Application Data
(60) Provisional application No. 61/154,738, filed on Feb. 23, 2009.

(51) Int. Cl. A61K 31/5377; C07D 413/14; C07D 401/14; C07D 405/14; A61P 31/14; A61K 31/496; A61K 31/4439; A61K 31/4184; A61K 31/4709; C07D 403/14; A61K 31/4178

(52) U.S. Cl. 514/232.2; 548/314.7; 544/82; 544/357; 548/313.1; 544/139; 544/370; 546/274.1; 548/306.1; 546/272.7; 548/302.1; 546/167; 514/397; 514/252.11; 514/235.8; 514/254.05; 514/341; 514/394; 514/393; 514/314

(57) ABSTRACT

Provided herein are compounds, pharmaceutical compositions and combination therapies for inhibition of hepatitis C.

### STATEMENT OF RELATED APPLICATIONS

This application claims the benefit of U.S. provisional applications 61/154,738 filed Feb. 23, 2009.

### FIELD OF THE INVENTION

The invention relates to compounds useful for inhibiting hepatitis C virus (“HCV”) replication, particularly functions of the non-structural 5A (“NS5A”) protein of HCV.

### BACKGROUND OF THE INVENTION

HCV is a single-stranded RNA virus that is a member of the Flaviviridae family. The virus shows extensive genetic heterogeneity as there are currently seven identified genotypes and more than 50 identified subtypes. In HCV infected cells, viral RNA is translated into a polyprotein that is cleaved into ten individual proteins. At the amino terminus are structural proteins: the core (C) protein and the envelope glycoproteins, E1 and E2, and p′7, an integral membrane protein that follows E1 and E2. Additionally, there are six non-structural proteins, NS2, NS3, NS4A, NS4B, NS5A and NS5B, which play a functional role in the HCV lifecycle. (see, for example, Lindenbach, B. D. and Rice, C. M. Nature. 436:933-938, 2005).

Infection by HCV is a serious health issue. It is estimated that 170 million people worldwide are chronically infected with HCV. HCV infection can lead to chronic hepatitis, cirrhosis, liver failure and hepatocellular carcinoma. Chronic HCV infection is thus a major worldwide cause of liver-related premature mortality.

The present standard of care treatment regimen for HCV infection involves interferon-alpha, alone or in combination with ribavirin. The treatment is cumbersome and sometimes has debilitating and severe side effects and many patients do not durably respond to treatment. New and effective methods of treating HCV infection are urgently needed.

### SUMMARY OF THE INVENTION

Essential features of the NS5A protein of HCV make it an ideal target for inhibitors. The present disclosure describes a class of compounds targeting the NS5A protein and methods of their use to treat HCV infection in humans.

In a first aspect, compounds of formula I are provided:

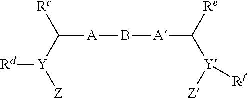

wherein,
A and A′ are independently selected from the group consisting of a single bond, —(CR₂)ₙ—C(O)—(CR₂)ₚ—, —(CR₂)ₙ—O—(CR₂)ₚ—, —(CR₂)ₙ—N(R^{N})—(CR₂)ₚ—, —(CR₂)ₙ—S(O)ₖ—(CR₂)ₚ—, —(CR₂)ₙ—S(O)ₖ—N(R^{N})—(CR₂)ₚ—, —(CR₂)ₙ—C(O)—N(R^{N})—(CR₂)ₚ—, —(CR₂)ₙ—N(R^{N})—C(O)—N(R^{N})—(CR₂)ₚ—, —(CR₂)ₙ—C(O)—O—(CR₂)ₚ—, —(CR₂)ₙ—N(R^{N})—S(O)ₖ—N(R^{N})—(CR₂)ₚ— and —(CR₂)ₙ—N(R^{N})—C(O)—O—(CR₂)ₚ— and a heteroaryl group selected from the group consisting of

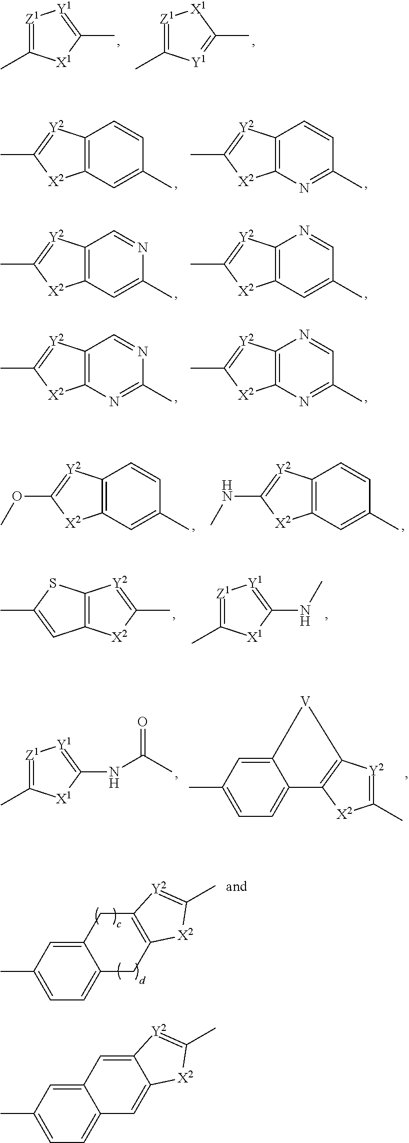

wherein:
   X¹ is CH₂, NH, O or S,
   Y¹, Y² and Z¹ are each independently CH or N,
   X² is NH, O or S,
   V is —CH₂—CH₂—, —CH═CH—, —N═CH—, (CH₂)ₐ—N(R^{N})—(CH₂)_{b}— or —(CH₂)ₐ—O—(CH₂)_{b}—, wherein a and b are independently 0, 1, 2 or 3 with the proviso that a and b are not both 0,

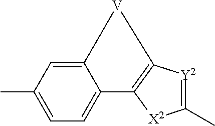

optionally includes 1 or 2 nitrogens as heteroatoms on the phenyl residue,
the carbons of the heteroaryl group are each independently optionally substituted with a substituent selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
the nitrogens, if present, of the heteroaryl group are each independently optionally substituted with a substituent selected from the group consisting of —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
a and b are independently 1, 2 or 3.
c and d are independently 1 or 2,
n and p are independently 0, 1, 2 or 3,
k is 0, 1 or 2,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
each R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
wherein for each A and A′, B may be attached to either side of A and A′ so that in the example of A or A′ being

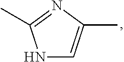

the A-B-A′ can be any of:

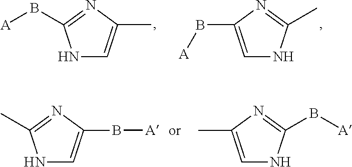

and wherein only one of A and A′ is a 5-membered heteroaryl ring if B is W—W;
B is W—W or W—X″—W wherein:
   each W is an aryl group or a heteroaryl group and X″ is selected from the group consisting of —O—, —S(O)ₖ, —N(R^{N})— and —CR′₂—,
   each R′ is independently selected from the group consisting of hydrogen, —OH, —CN, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino and the two R′ are optionally joined to form a 3- to 8-membered ring, and
   each W is independently optionally substituted with one or more substituents each independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
R^{c}, R^{d}, R^{e} and R^{f} are each independently selected from the group consisting of: hydrogen, C₁ to C₈ alkyl, C₁ to C₈ heteroalkyl, aralkyl and a 4- to 8-membered ring which may be cycloalkyl, heterocycle, heteroaryl or aryl, wherein,
   each hetero atom, if present, is independently N, O or S,
   each of R^{c}, R^{d}, R^{e} and R^{f} may optionally be substituted by C₁ to C₈ alkyl, C₁ to C₈ heteroalkyl, aralkyl or a 4- to 8-membered ring which may be cycloalkyl, heterocycle, heteroaryl or aryl and wherein each heteroatom, if present, is independently N, O or S,
R^{c} and R^{d} are optionally joined to form a 4- to 8-membered heterocycle which is optionally fused to another 3- to 6-membered heterocycle or heteroaryl ring, and
R^{e} and R^{f} are optionally joined to form a 4- to 8-membered heterocycle which is optionally fused to another 3- to 6-membered heterocycle or heteroaryl ring;
Y and Y′ are independently carbon or nitrogen; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the first aspect, B is W—W.

In a second embodiment of the first aspect, B is selected from the group consisting of

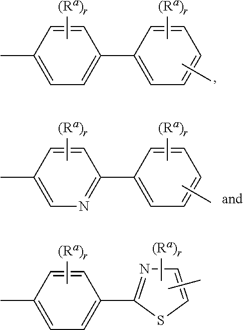

wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

In a third embodiment of the first aspect, B is W—X″—W.

In a fourth embodiment of the first aspect, B is W—S—W.

In a fifth embodiment of the first aspect, B is W—O—W.

In a sixth embodiment of the first aspect, B is selected from the group consisting of

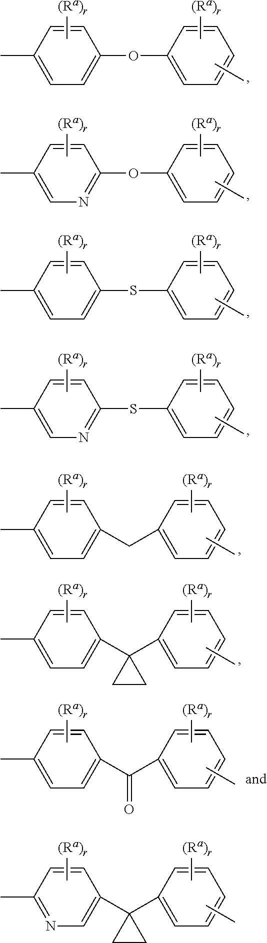

wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

In a seventh embodiment of the first aspect, A is selected from the group consisting of a single bond, —(CR₂)ₙ—O—(CR₂)ₚ—, —(CR₂)ₙ—N(R^{N})—(CR₂)ₚ—, —(CR₂)ₙ—C(O)—N(R^{N})—(CR₂)ₚ—, —(CR₂)ₙ—N(R^{N})—C(O)—N(R^{N})—(CR₂)_{P}—, —(CR₂)ₙ—S(O)ₖ—(CR₂)ₚ— and —(CR₂)ₙ—N(R^{N})—C(O)—O—(CR₂)ₚ—.

In an eighth embodiment of the first aspect, A is —(CR₂)ₙ—O—(CR₂)ₚ— or —(CR₂)ₙ—C(O)—N(R^{N})—(CR₂)ₚ—.

In a ninth embodiment of the first aspect, A′ is selected from the group consisting

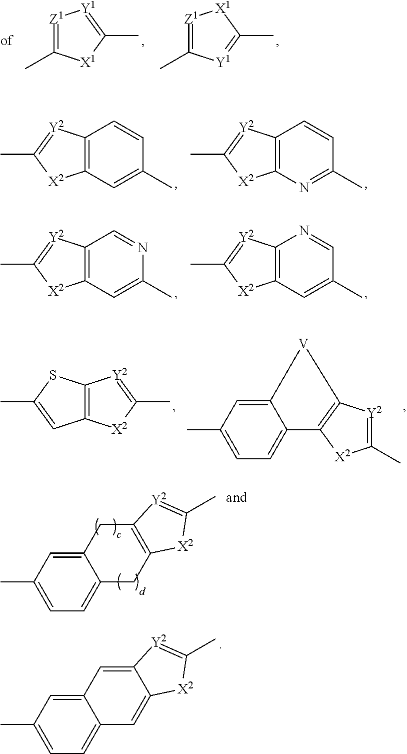

In a tenth embodiment of the first aspect, A′ is selected from the group consisting

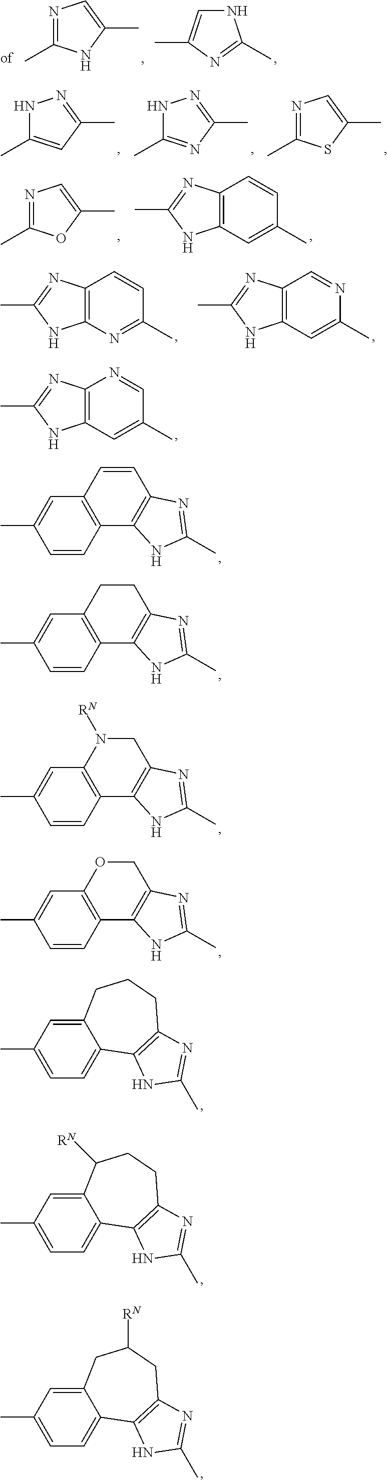

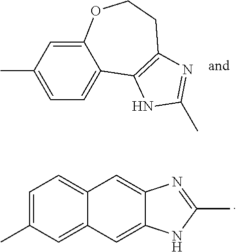

In an eleventh embodiment of the first aspect, A′ is selected from the group consisting of

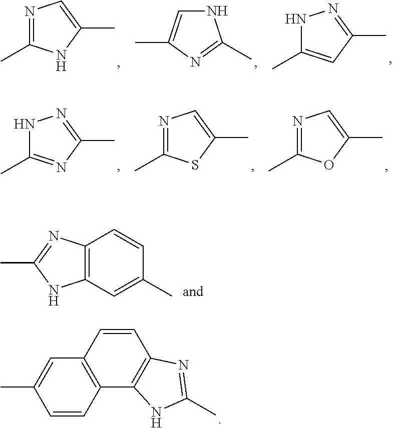

In a twelfth embodiment of the first aspect each W is independently optionally substituted with —CN, —OCF₃, —OCHF₂, —CF₃ or —F.

In a thirteenth embodiment of the first aspect, R^{c}, R^{d}, R^{e} and R^{f} are each independently selected from the group consisting of: hydrogen, C₁ to C₈ alkyl and C₁ to C₈ heteroalkyl, wherein,
each hetero atom, if present, is independently N, O or S,
R^{c} and R^{d} are optionally joined to form a 4- to 8-membered heterocycle which is optionally fused to another 3- to 6-membered heterocycle, and
R^{e} and R^{f} are optionally joined to form a 4- to 8-membered heterocycle which is optionally fused to another 3- to 6-membered heterocycle.

In a fourteenth embodiment of the first aspect one or both of R^{c} and R^{d} or R^{e} and R^{f} are optionally joined to form a 4- to 8-membered heterocycle which is optionally fused to another 3- to 6-membered heterocycle.

In a fifteenth embodiment of the first aspect R^{c} and R^{d} are joined and form a heterocyclic fused ring system selected from the group consisting of:

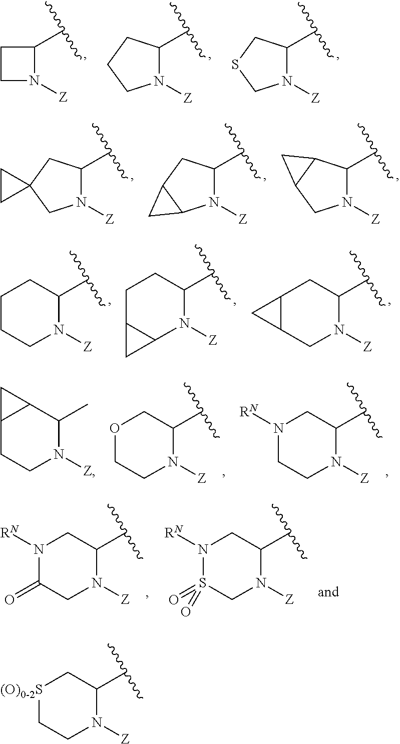

wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide.

In a sixteenth embodiment of the first aspect R^{c} and R^{d} are joined and form one of

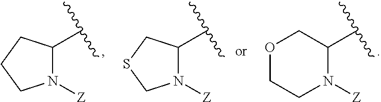

In a seventeenth embodiment of the first aspect R^{e} and R^{f} are joined and form a heterocyclic fused ring system selected from the group consisting of:

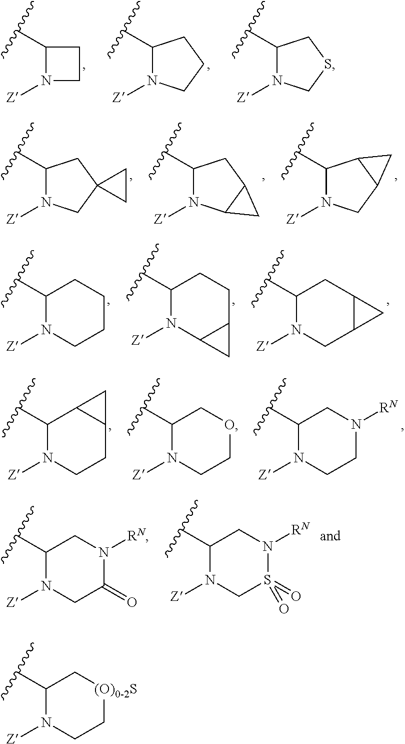

wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide.

In an eighteenth embodiment of the first aspect R^{e} and R^{f} are joined and form

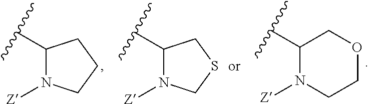

In a nineteenth embodiment of the first aspect one of Y and Y′ is N.

In a twentieth embodiment of the first aspect both Y and Y′ are N.

In a second aspect of the invention, compounds of formula II are provided:

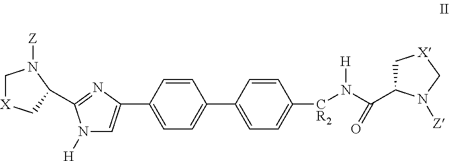

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the second aspect, one R is hydrogen and one R is —CH₃.

In a third aspect of the invention, compounds of formula III are provided:

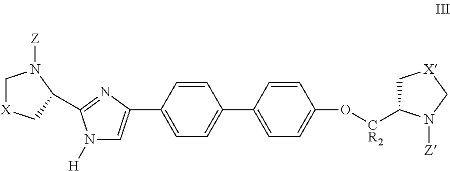

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the third aspect, one R is hydrogen and one R is —CH₃.

In a fourth aspect compounds formula IV are provided:

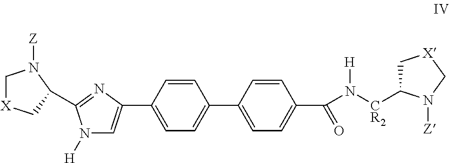

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the fourth aspect one R is hydrogen and one R is —CH₃.

In a fifth aspect of the invention, compounds of formula V are provided:

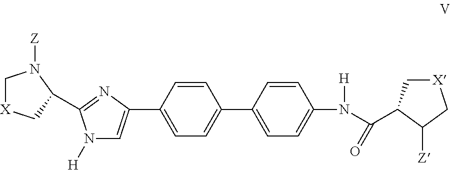

wherein,
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a sixth aspect of the invention, compounds of formula VI:

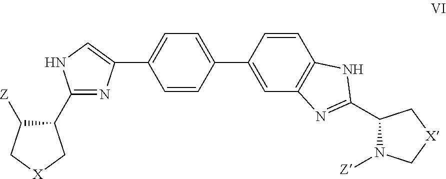

wherein,
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C_{8 alkyl, C}₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a seventh aspect of the invention, compounds of the following formulae are provided:

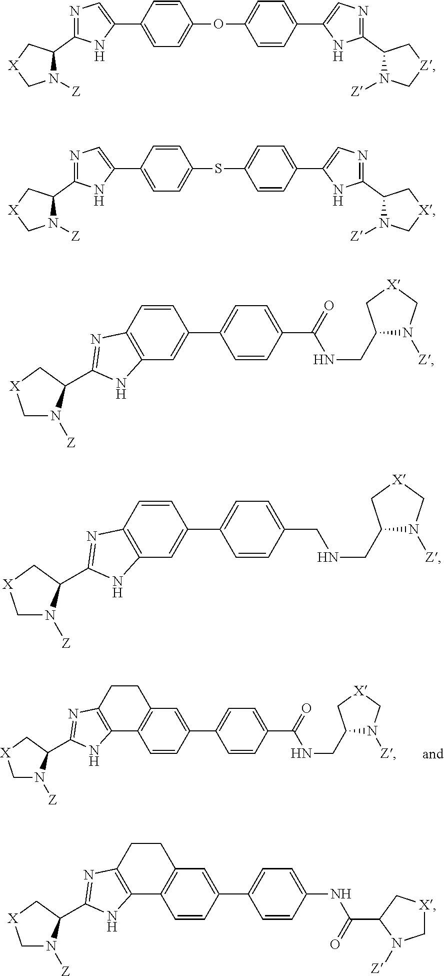

wherein:
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In an eighth aspect of the invention Z and Z′ in any of the previous aspects are each 1-3 amino acids.

In a first embodiment of the eighth aspect, the amino acids are in the D configuration.

In a ninth aspect of the invention, Z and Z′ are each independently selected from the group consisting of —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸.

In a first embodiment of the ninth aspect, one or both of Z and Z′ are —[U—(CR⁴₂)ₜ—NR⁵—(CR⁴₂)ₜ]ᵤ—U—(CR⁴₂)ₜ—NR⁷—(CR⁴₂)ₜ—R⁸.

In a second embodiment of the ninth aspect, one or both of Z and Z′ are —U—(CR⁴₂)ₜ—NR⁵—(CR⁴₂)ₜ—U—(CR⁴₂)ₜ—NR⁷—(CR⁴₂)ₜ—R⁸.

In a third embodiment of the ninth aspect, one or both of Z and Z′ are —U—(CR⁴₂)ₜ—NR⁷—(CR⁴₂)ₜ—R⁸.

In a fourth embodiment of the ninth aspect, one or both of Z and Z′ are —[C(O)—(CR⁴₂)ₜ—NR⁵—(CR⁴₂)ₜ]ᵤ—U—(CR⁴₂)ₜ—NR⁷—(CR⁴₂)ₜ—R⁸.

In a fifth embodiment of the ninth aspect, one or both of Z and Z′ are —C(O)—(CR⁴₂)ₜ—NR⁵—(CR⁴₂)ₜ—U—(CR⁴₂)ₜ—NR⁷—(CR⁴₂)ₜ—R⁸.

In a sixth embodiment of the ninth aspect, one or both of Z and Z′ are —[C(O)—(CR⁴₂)ₜ—NR⁵—(CR⁴₂)ₜ]ᵤ—C(O)—(CR⁴₂)ₜ—NR⁷—(CR⁴₂)ₜ—R⁸.

In a seventh embodiment of the ninth aspect, one or both of Z and Z′ are —C(O)—(CR⁴₂)ₜ—NR⁵—(CR⁴₂)ₜ—C(O)—(CR⁴₂)ₜ—NR⁷—(CR⁴₂)ₜ—R⁸.

In an eighth embodiment of the ninth aspect, one or both of Z and Z′ are —C(O)—(CR⁴₂)ₜ—NR⁷—(CR⁴₂)ₜ—R⁸.

In a ninth embodiment of the ninth aspect, one or both of Z and Z′ are —C(O)—(CR⁴₂)ₙ—NR⁷—(CR⁴₂)ₙ—C(O)—R⁸¹.

In a tenth embodiment of the ninth aspect, one or both of Z and Z′ are —C(O)—(CR⁴₂)ₙ—NR⁷—C(O)—R⁸¹.

In an eleventh embodiment of the ninth aspect, one or both of Z and Z′ are —C(O)—(CR⁴₂)ₙ—NR⁷—(CR⁴₂)ₙ—C(O)—O—R⁸¹.

In a twelfth embodiment of the ninth aspect, one or both of Z and Z′ are —C(O)—(CR⁴₂)ₙ—NR⁷—C(O)—O—R⁸¹.

In a thirteenth embodiment of the ninth aspect, one or both of Z and Z′ are —U—(CR⁴₂)ₜ—R⁸.

In a fourteenth embodiment of the ninth aspect, one or both of Z and Z′ are —C(O)—(CR⁴₂)ₜ—R⁸.

In a fifteenth embodiment of the ninth aspect, one or both of Z and Z′ are —[U—(CR⁴₂)ₜ—NR⁵—(CR⁴₂)ₜ]ᵤ—U—(CR⁴₂)ₜ—O—(CR⁴₂)ₜ—R⁸.

In a sixteenth embodiment of the ninth aspect, one or both of Z and Z′ are —U—(CR⁴₂)ₜ—NR⁵—(CR⁴₂)ₜ—U—(CR⁴₂)ₜ—O—(CR⁴₂)ₜ—R⁸.

In a seventeenth embodiment of the ninth aspect, one or both of Z and Z′ are —C(O)—(CR⁴₂)ₜ—NR⁵—(CR⁴₂)ₜ—C(O)—(CR⁴₂)ₜ—O—(CR⁴₂)ₜ—R⁸.

In an eighteenth embodiment of the ninth aspect, one or both of Z and Z′ are —U—(CR⁴₂)ₜ—O—(CR⁴₂)ₜ—R⁸.

In a nineteenth embodiment of the ninth aspect, one or both of Z and Z′ are —C(O)—(CR⁴₂)ₜ—O—(CR⁴₂)ₜ—R⁸.

In a twentieth embodiment of the ninth aspect, one or both of Z and Z′ are —C(O)—(CR⁴₂)ₙ—NR⁷—R⁸ wherein R⁷ and R⁸ together form a 4-7 membered ring.

A tenth aspect of the invention provides a pharmaceutical composition comprising the compounds of the invention.

An eleventh aspect of the invention provides use of the compounds of the invention in the manufacture of a medicament.

In a first embodiment of the eleventh aspect the medicament is for the treatment of hepatitis C.

A twelfth aspect of the invention provides a method of treating hepatitis C comprising administering to a subject in need thereof, a therapeutically effective amount of any one of the compounds of the invention.

### DETAILED DESCRIPTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (2007) “Advanced Organic Chemistry 5^{th} Ed.” Vols. A and B, Springer Science+Business Media LLC, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology.

The term “alkanoyl” as used herein contemplates a carbonyl group with a lower alkyl group as a substituent.

The term “alkenyl” as used herein contemplates substituted or unsubstituted, straight and branched chain alkene radicals, including both the E- and Z-forms, containing from two to eight carbon atoms. The alkenyl group may be optionally substituted with one or more substituents selected from the group consisting of halogen, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —S(O)R, —SO₂R, —SO₃R, —S(O)₂N(R^{N})₂, phosphate, phosphonate, cycloalkyl, cycloalkenyl, aryl and heteroaryl.

The term “alkoxy” as used herein contemplates an oxygen with a lower alkyl group as a substituent and includes methoxy, ethoxy, butoxy, trifluoromethoxy and the like. It also includes divalent substituents linked to two separated oxygen atoms such as, without limitation, —O—(CH₂)₁₋₄—O—, —O—CF₂—O—, —O—(CH₂)₁₋₄—O—(CH₂CH₂—O)₁₋₄— and —(O—CH₂CH₂—O)₁₋₄—.

The term “alkoxycarbonyl” as used herein contemplates a carbonyl group with an alkoxy group as a substituent.

The term “alkyl” as used herein contemplates substituted or unsubstituted, straight and branched chain alkyl radicals containing from one to fifteen carbon atoms. The term “lower alkyl” as used herein contemplates both straight and branched chain alkyl radicals containing from one to six carbon atoms and includes methyl, ethyl, propyl, isopropyl, butyl, isobutyl, tert-butyl and the like. The alkyl group may be optionally substituted with one or more substituents selected from halogen, —CN, —NO₂, —C(O)₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The term “alkylene,” “alkenylene” and “alkynylene” as used herein refers to the groups “alkyl,” “alkenyl” and “alkynyl” respectively, when they are divalent, ie, attached to two atoms.

The term “alkylsulfonyl” as used herein contemplates a sulfonyl group which has a lower alkyl group as a substituent.

The term “alkynyl” as used herein contemplates substituted or unsubstituted, straight and branched carbon chain containing from two to eight carbon atoms and having at least one carbon-carbon triple bond. The term alkynyl includes, for example ethynyl, 1-propynyl, 2-propynyl, 1-butynyl, 3-methyl-1-butynyl and the like. The alkynyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The term “amino” as used herein contemplates a group of the structure —NR^{N}₂.

The term “amino acid” as used herein contemplates a group of the structure

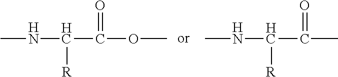

in either the D or the L configuration and includes but is not limited to the twenty “standard” amino acids: isoleucine, leucine, lysine, methionine, phenylalanine, threonine, tryptophan, valine, alanine, asparagine, aspartate, cysteine, glutamate, glutamine, glycine, proline, serine, tyrosine, arginine and histidine. The present invention also includes, without limitation, D-configuration amino acids, beta-amino acids, amino acids having side chains as well as all non-natural amino acids known to one skilled in the art.

The term “aralkyl” as used herein contemplates a lower alkyl group which has as a substituent an aromatic group, which aromatic group may be substituted or unsubstituted. The aralkyl group may be optionally substituted with one or more substituents selected from halogen, —CN, —NO₂, —CO₂R, —C(O)R, —O—R, —N(R^{N})₂, —N(R^{N})C(O)R, —N(R^{N})S(O)₂R, —SR, C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The terms “aryl,” “aromatic group” or “aromatic ring” as used herein contemplates substituted or unsubstituted single-ring and multiple aromatic groups (for example, phenyl, pyridyl and pyrazole, etc.) and polycyclic ring systems (naphthyl and quinolinyl, etc.). The polycyclic rings may have two or more rings in which two atoms are common to two adjoining rings (the rings are “fused”) wherein at least one of the rings is aromatic, e.g., the other rings can be cycloalkyls, cycloalkenyls, aryl, heterocycles and/or heteroaryls. The aryl group may be optionally substituted with one or more substituents selected from halogen, alkyl, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, —SiR₃, —P(O)R, phosphate, phosphonate, cycloalkyl, cycloalkenyl, aryl and heteroaryl.

The term “arylsulfonyl” as used herein contemplates a sulfonyl group which has as a substituent an aryl group. The term is meant to include, without limitation, monovalent as well as multiply valent aryls (eg, divalent aryls).

The term “carbamoyl” as used herein contemplates a group of the structure

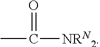

The term “carbonyl” as used herein contemplates a group of the structure

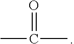

The term “carboxyl” as used herein contemplates a group of the structure

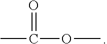

The term “cycloalkyl” as used herein contemplates substituted or unsubstituted cyclic alkyl radicals containing from three to twelve carbon atoms and includes cyclopropyl, cyclopentyl, cyclohexyl and the like. The term “cycloalkyl” also includes polycyclic systems having two rings in which two or more atoms are common to two adjoining rings (the rings are “fused”). The cycloalkyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —S(O)₂N(R^{N})₂, phosphate, phosphonate, alkyl, cycloalkenyl, aryl and heteroaryl.

The term “cycloalkenyl” as used herein contemplates substituted or unsubstituted cyclic alkenyl radicals containing from four to twelve carbon atoms in which there is at least one double bond between two of the ring carbons and includes cyclopentenyl, cyclohexenyl and the like. The term “cycloalkenyl” also includes polycyclic systems having two rings in which two or more atoms are common to two adjoining rings (the rings are “fused”). The cycloalkenyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —S(O)₂N(R^{N})₂, phosphate, phosphonate, alkyl, cycloalkenyl, aryl and heteroaryl.

The term “halo” or “halogen” as used herein includes fluorine, chlorine, bromine and iodine.

The term “heteroalkyl” as used herein contemplates an alkyl with one or more heteroatoms.

The term “heteroatom”, particularly within a ring system, refers to N, O and S.

The term “heterocyclic group,” “heterocycle” or “heterocyclic ring” as used herein contemplates substituted or unsubstituted aromatic and non-aromatic cyclic radicals having at least one heteroatom as a ring member. Preferred heterocyclic groups are those containing five or six ring atoms which includes at least one hetero atom and includes cyclic amines such as morpholino, piperidino, pyrrolidino and the like and cyclic ethers, such as tetrahydrofuran, tetrahydropyran and the like. Aromatic heterocyclic groups, also termed “heteroaryl” groups, contemplates single-ring hetero-aromatic groups that may include from one to three heteroatoms, for example, pyrrole, furan, thiophene, imidazole, oxazole, thiazole, triazole, pyrazole, oxodiazole, thiadiazole, pyridine, pyrazine, pyridazine, pyrimidine and the like. The term heteroaryl also includes polycyclic hetero-aromatic systems having two or more rings in which two or more atoms are common to two adjoining rings (the rings are “fused”) wherein at least one of the rings is a heteroaryl, e.g., the other rings can be cycloalkyls, cycloalkenyls, aryl, heterocycles and/or heteroaryls. Examples of polycyclic heteroaromatic systems include quinoline, isoquinoline, cinnoline, tetrahydroisoquinoline, quinoxaline, quinazoline, benzimidazole, benzofuran, benzothiophene, benzoxazole, benzothiazole, indazole, purine, benzotriazole, pyrrolepyridine, pyrrazolopyridine and the like. The heterocyclic group may be optionally substituted with one or more substituents selected from the group consisting of halo, alkyl, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, —SiR₃, —P(O)R, phosphate, phosphonate, cycloalkyl, cycloalkenyl, aryl and heteroaryl.

The term “oxo” as used herein contemplates an oxygen atom attached with a double bond.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

“Pharmaceutically acceptable salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19).

The terms “phosphate” and “phosphonate” as used herein refer to the moieties having the following structures, respectively:

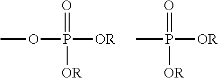

The terms “salts” and “hydrates” refers to the hydrated forms of the compound that would favorably affect the physical or pharmacokinetic properties of the compound, such as solubility, palatability, absorption, distribution, metabolism and excretion. Other factors, more practical in nature, which those skilled in the art may take into account in the selection include the cost of the raw materials, ease of crystallization, yield, stability, solubility, hygroscopicity, flowability and manufacturability of the resulting bulk drug.

The term sulfonamide as used herein contemplates a group having the structure

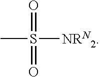

The term “sulfonate” as used herein contemplates a group having the structure

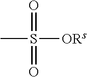

wherein R^{s} is selected from the group consisting of hydrogen, C₁-C₁₀ alkyl, C₂-C₁₀ alkenyl, C₂-C₁₀ alkynyl, C₁-C₁₀ alkanoyl or C₁-C₁₀ alkoxycarbonyl.

The term “sulfonyl” as used herein contemplates a group having the structure

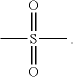

“Substituted sulfonyl” as used herein contemplates a group having the structure

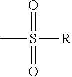

including, but not limited to alkylsulfonyl and arylsulfonyl.

The term “thiocarbonyl,” as used herein, means a carbonyl wherein an oxygen atom has been replaced with a sulfur.

Each R is independently selected from hydrogen, —OH, —CN, —NO₂, halogen, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, substituted sulfonyl, sulfonate, sulfonamide, amino and oxo.

Each R^{N} is independently selected from the group consisting of hydrogen, —OH, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, substituted sulfonyl, sulfonate and sulfonamide. Two R^{N} may be taken together with C, O, N or S to which they are attached to form a five to seven membered ring which may optionally contain a further heteroatom.

The compounds of the present invention may be used to inhibit or reduce the activity of HCV, particularly HCV's NS5A protein. In these contexts, inhibition and reduction of activity of the NS5A protein refers to a lower level of the measured activity relative to a control experiment in which the cells or the subjects are not treated with the test compound. In particular aspects, the inhibition or reduction in the measured activity is at least a 10% reduction or inhibition. One of skill in the art will appreciate that reduction or inhibition of the measured activity of at least 20%, 50%, 75%, 90% or 100% or any number in between, may be preferred for particular applications.

In a first aspect, compounds of formula I are provided:

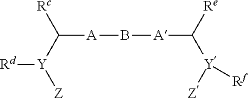

wherein,
A and A′ are independently selected from the group consisting of a single bond, —(CR₂)ₙ—C(O)—(CR₂)ₚ—, —(CR₂)ₙ—O—(CR₂)ₚ—, —(CR₂)ₙ—N(R^{N})—(CR₂)ₚ—, —(CR₂)ₙ—S(O)ₖ—(CR₂)ₚ—, —(CR₂)ₙ—S(O)ₖ—N(R^{N})—(CR₂)ₚ—, —(CR₂)ₙ—C(O)—N(R^{N})—(CR₂)ₚ—, —(CR₂)ₙ—N(R^{N})—C(O)—N(R^{N})—(CR₂)ₚ—, —(CR₂)ₙ—C(O)—O—(CR₂)ₚ—, —(CR₂)ₙ—N(R^{N})—S(O)ₖ—N(R^{N})—(CR₂)ₚ— and —(CR₂)ₙ—N(R^{N})—C(O)—O—(CR₂)ₚ— and a heteroaryl group selected from the group consisting of

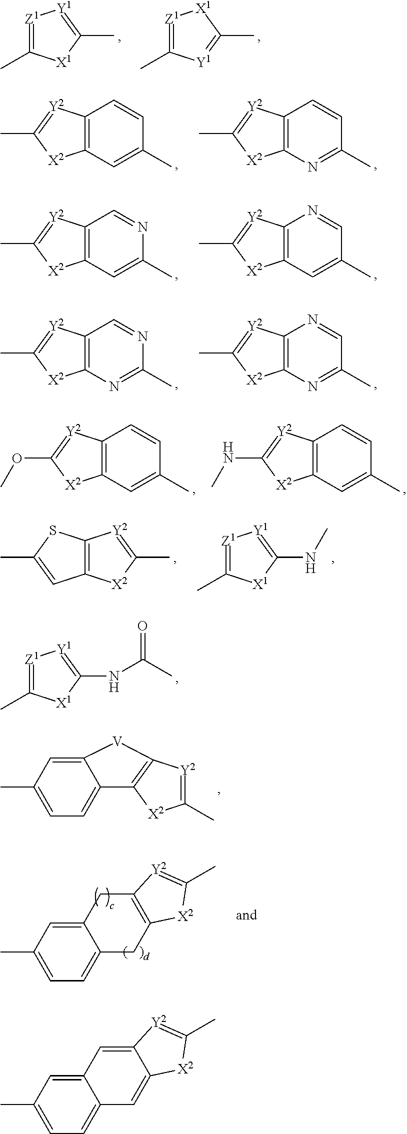

wherein:
   Xⁱ is CH₂, NH, O or S,
   Y¹, Y² and Z¹ are each independently CH or N,
   X² is NH, O or S,
   V is —CH₂—CH₂—, —CH═CH—, —N═CH—, (CH₂)ₐ—N(R^{N})—(CH₂)_{b}— or —(CH₂)ₐ—O—(CH₂)_{b}—, wherein a and b are independently 0, 1, 2 or 3 with the proviso that a and b are not both 0,

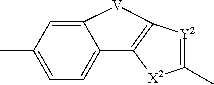

optionally includes 1 or 2 nitrogens as heteroatoms on the phenyl residue,
the carbons of the heteroaryl group are each independently optionally substituted with a substituent selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
the nitrogens, if present, of the heteroaryl group are each independently optionally substituted with a substituent selected from the group consisting of —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
a and b are independently 1, 2 or 3.
c and d are independently 1 or 2,
n and p are independently 0, 1, 2 or 3,
k is 0, 1 or 2,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
each R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
wherein for each A and A′, B may be attached to either side of A and A′ so that in the example of A or A′ being

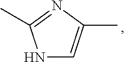

the A-B-A′ can be any of:

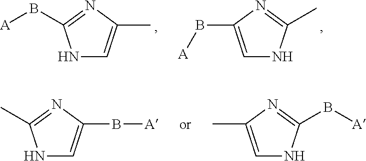

and wherein only one of A and A′ is a 5-membered heteroaryl ring if B is W—W;
B is W—W or W—X″—W wherein:
   each W is an aryl group or a heteroaryl group and X″ is selected from the group consisting of —O—, —S(O)ₖ, —N(R^{N})— and —CR′₂—,
   each R′ is independently selected from the group consisting of hydrogen, —OH, —CN, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino and the two R′ are optionally joined to form a 3- to 8-membered ring, and
   each W is independently optionally substituted with one or more substituents each independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
R^{c}, R^{d}, R^{e} and R^{f} are each independently selected from the group consisting of: hydrogen, C₁ to C₈ alkyl, C₁ to C₈ heteroalkyl, aralkyl and a 4- to 8-membered ring which may be cycloalkyl, heterocycle, heteroaryl or aryl, wherein,
   each hetero atom, if present, is independently N, O or S,
each of R^{c}, R^{d}, R^{e} and R^{f} may optionally be substituted by C₁ to C₈ alkyl, C₁ to C₈ heteroalkyl, aralkyl or a 4- to 8-membered ring which may be cycloalkyl, heterocycle, heteroaryl or aryl and wherein each heteroatom, if present, is independently N, O or S,
   R^{c} and R^{d} are optionally joined to form a 4- to 8-membered heterocycle which is optionally fused to another 3- to 6-membered heterocycle or heteroaryl ring, and
   R^{e} and R^{f} are optionally joined to form a 4- to 8-membered heterocycle which is optionally fused to another 3- to 6-membered heterocycle or heteroaryl ring;
Y and Y′ are independently carbon or nitrogen; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the first aspect, B is W—W.

In a second embodiment of the first aspect, B is selected from the group consisting of

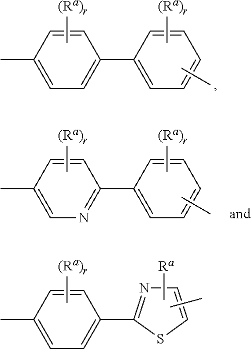

wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

In a third embodiment of the first aspect, B is W—X″—W.

In a fourth embodiment of the first aspect, B is W—S—W.

In a fifth embodiment of the first aspect, B is W—O—W. In a sixth embodiment of the first aspect, B is selected from the group consisting of

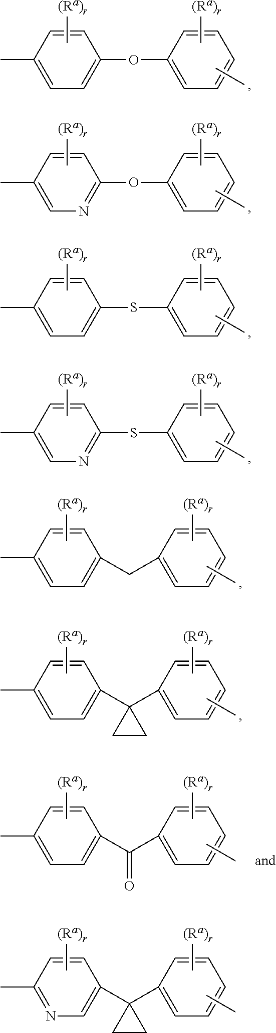

wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

In a seventh embodiment of the first aspect, A is selected from the group consisting of a single bond, —(CR₂)ₙ—O—(CR₂)ₚ—, —(CR₂)ₙ—N(R^{N})—(CR₂)ₚ—, —(CR₂)ₙ—C(O)—N(R^{N})—(CR₂)ₚ—, —(CR₂)ₙ—N(R^{N})—C(O)—N(R^{N})—(CR₂)ₚ—, —(CR₂)ₙ—S(O)ₖ—(CR₂)ₚ— and —(CR₂)ₙ—N(R^{N})—C(O)—O—(CR₂)ₚ—.

In an eighth embodiment of the first aspect, A is —(CR₂)ₙ—O—(CR₂)ₚ— or —(CR₂)ₙ—C(O)—N(R^{N})—(CR₂)ₚ—.

In a ninth embodiment of the first aspect, A′ is selected from the group consisting of

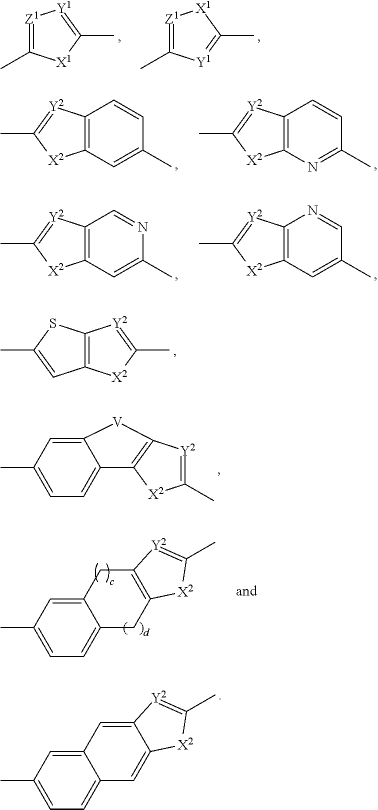

In a tenth embodiment of the first aspect, A′ is selected from the group consisting of

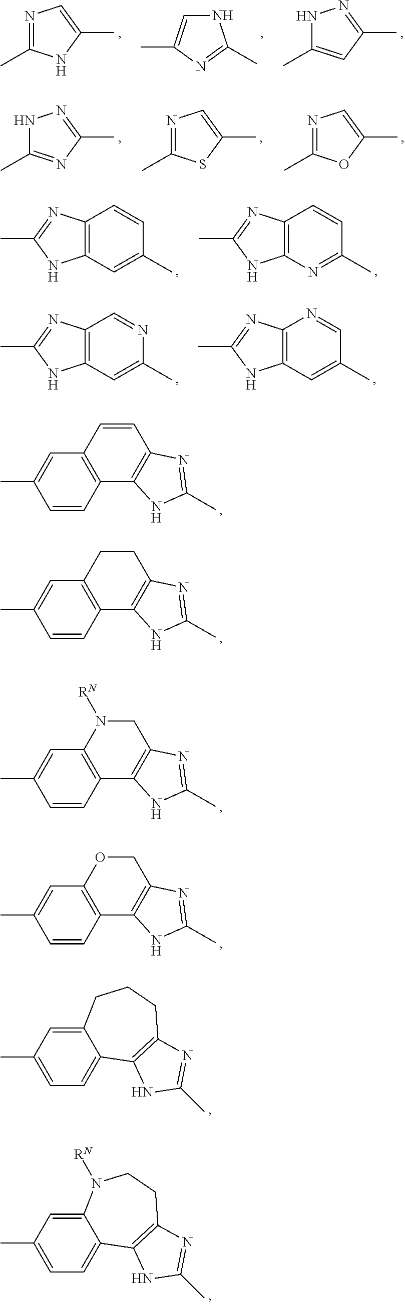

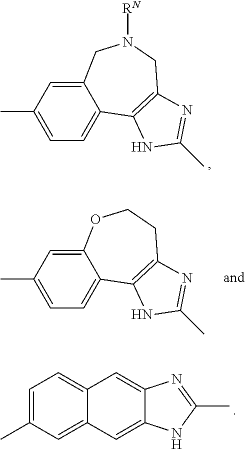

In an eleventh embodiment of the first aspect, A′ is selected from the group consisting of

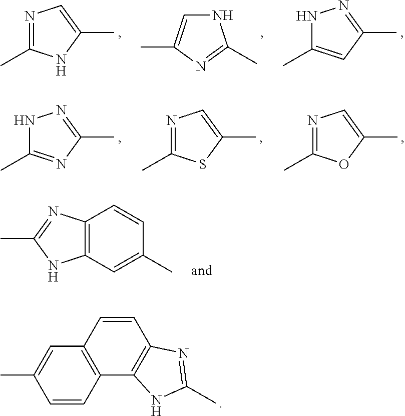

In a twelfth embodiment of the first aspect each W is independently optionally substituted with —CN, —OCF₃, —OCHF₂, —CF₃ or —F.

In a thirteenth embodiment of the first aspect, R^{c}, R^{d}, R^{e} and R^{f} are each independently selected from the group consisting of: hydrogen, C₁ to C₈ alkyl and C₁ to C₈ heteroalkyl, wherein,
each hetero atom, if present, is independently N, O or S,
R^{c} and R^{d} are optionally joined to form a 4- to 8-membered heterocycle which is optionally fused to another 3- to 6-membered heterocycle, and
R^{e} and R^{f} are optionally joined to form a 4- to 8-membered heterocycle which is optionally fused to another 3- to 6-membered heterocycle.

In a fourteenth embodiment of the first aspect one or both of R^{e} and R^{d} or R^{e} and R^{f} are optionally joined to form a 4- to 8-membered heterocycle which is optionally fused to another 3- to 6-membered heterocycle.

In a fifteenth embodiment of the first aspect R^{e} and R^{d} are joined and form a heterocyclic fused ring system selected from the group consisting of:

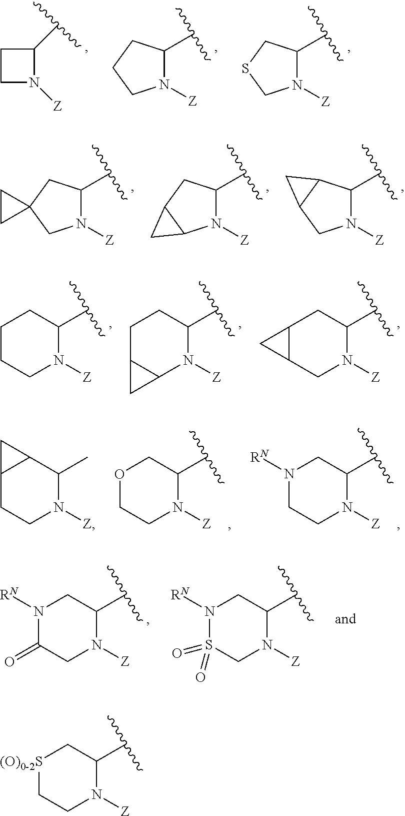

wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide.

In a sixteenth embodiment of the first aspect R^{c} and R^{d} are joined and form one of

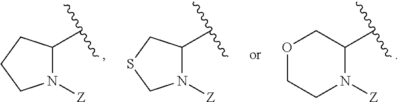

In a seventeenth embodiment of the first aspect R^{e} and R^{f} are joined and form a heterocyclic fused ring system selected from the group consisting of:

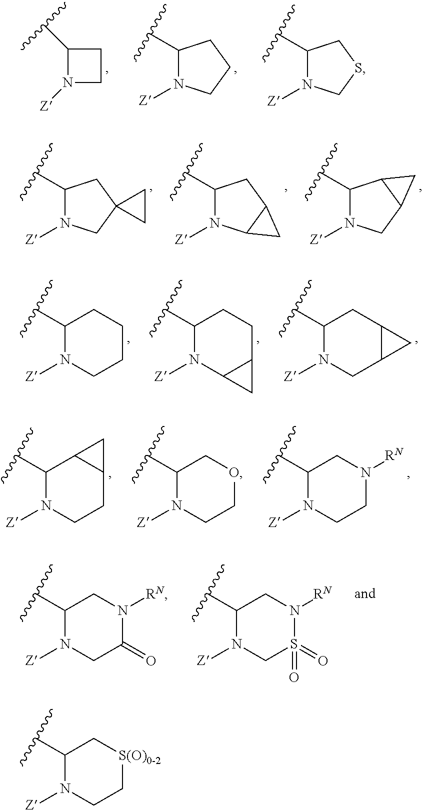

wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide.

In an eighteenth embodiment of the first aspect R^{e} and R^{f} are joined and form

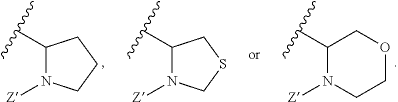

In a nineteenth embodiment of the first aspect one of Y and Y′ is N.

In a twentieth embodiment of the first aspect both Y and Y′ are N.

In a second aspect of the invention, compounds of formula II are provided:

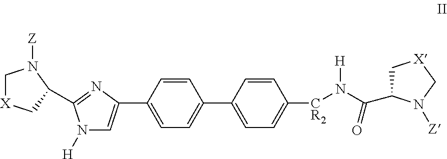

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the second aspect, one R is hydrogen and one R is —CH₃.

In a third aspect of the invention, compounds of formula III are provided:

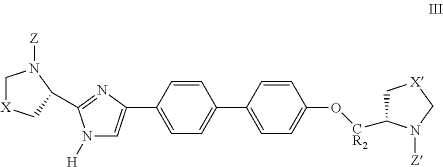

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the third aspect, one R is hydrogen and one R is —CH₃.

In a fourth aspect compounds formula IV are provided:

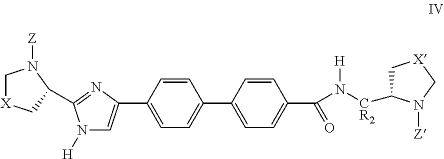

wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a first embodiment of the fourth aspect one R is hydrogen and one R is —CH₃.

In a fifth aspect of the invention, compounds of formula V are provided:

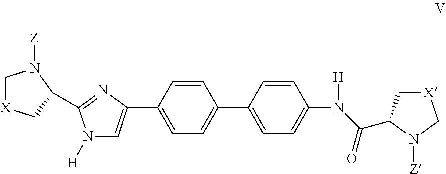

wherein,
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C_{8 alkyl, C}₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a sixth aspect of the invention, compounds of formula VI:

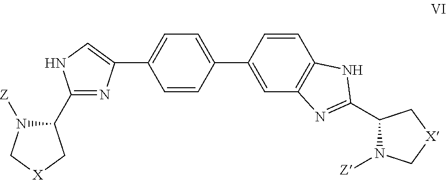

wherein,
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In a seventh aspect of the invention, compounds of the following formulae are provided:

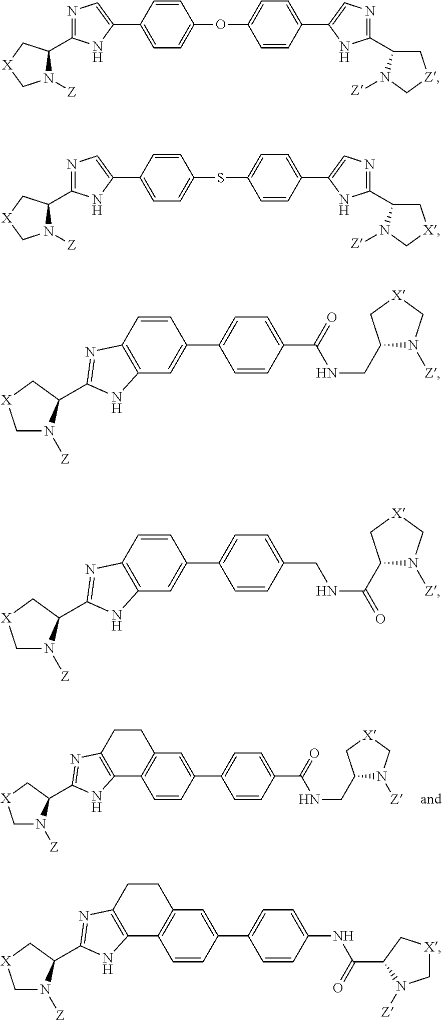

wherein:
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
   U is selected from the group consisting of —C(O)—, —C(S)— and —S(O)₂—,
   each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
   R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
   optionally, R⁷ and R⁸ together form a 4-7 membered ring,
   each t is independently 0, 1, 2, 3 or 4, and
   u is 0, 1 or 2.

In an eighth aspect of the invention Z and Z′ in any of the previous aspects are each 1-3 amino acids.

In a first embodiment of the eighth aspect, the amino acids are in the D configuration.

In a ninth aspect of the invention, Z and Z′ are each independently selected from the group consisting of —[U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸.

In a first embodiment of the ninth aspect, one or both of Z and Z′ are —[U—(CR⁴₂)ₜ—NR⁵—(CR⁴₂)ₜ]ᵤ—U—(CR⁴₂)ₜ—NR⁷—(CR⁴₂)ₜ—R⁸.

In a second embodiment of the ninth aspect, one or both of Z and Z′ are —U—(CR⁴₂)ₜ—NR⁵—(CR⁴₂)ₜ—U—(CR⁴₂)ₜ—NR⁷—(CR⁴₂)ₜ—R⁸.

In a third embodiment of the ninth aspect, one or both of Z and Z′ are —U—(CR⁴₂)ₜ—NR'—(CR⁴₂)ₜ—R⁸.

In a fourth embodiment of the ninth aspect, one or both of Z and Z′ are —[C(O)—(CR⁴₂)ₜ—NR⁵—(CR⁴₂)ₜ]ᵤ—U—(CR⁴₂)ₜ—NR⁷—(CR⁴₂)ₜ—R⁸.

In a fifth embodiment of the ninth aspect, one or both of Z and Z′ are —C(O)—(CR⁴₂)ₜ—NR⁵—(CR⁴₂)ₜ—U—(CR⁴₂)ₜ—NR⁷—(CR⁴₂)ₜ—R⁸.

In a sixth embodiment of the ninth aspect, one or both of Z and Z′ are —[C(O)—(CR⁴₂)ₜ—NR⁵—(CR⁴₂)ₜ]ᵤ—C(O)—(CR⁴₂)ₜ—NR⁷—(CR⁴₂)ₜ—R⁸.

In a seventh embodiment of the ninth aspect, one or both of Z and Z′ are —C(O)—(CR⁴₂)ₜ—NR⁵—(CR⁴₂)ₜ—C(O)—(CR⁴₂)ₜ—NR⁷—(CR⁴₂)ₜ—R⁸.

In an eighth embodiment of the ninth aspect, one or both of Z and Z′ are —C(O)—(CR⁴₂)ₜ—NR⁷—(CR⁴₂)ₜ—R⁸.

In a ninth embodiment of the ninth aspect, one or both of Z and Z′ are —C(O)—(CR⁴₂)ₙ—NR⁷—(CR⁴₂)ₙ—C(O)—R⁸¹.

In a tenth embodiment of the ninth aspect, one or both of Z and Z′ are —C(O)—(CR⁴₂)ₙ—NR⁷—C(O)—R⁸¹.

In an eleventh embodiment of the ninth aspect, one or both of Z and Z′ are —C(O)—(CR⁴₂)ₙ—NR⁷—(CR⁴₂)ₙ—C(O)—O—R⁸¹.

In a twelfth embodiment of the ninth aspect, one or both of Z and Z′ are —C(O)—(CR⁴₂)ₙ—NR⁷—C(O)—O—R⁸¹.

In a thirteenth embodiment of the ninth aspect, one or both of Z and Z′ are —U—(CR⁴₂)ₜ—R⁸.

In a fourteenth embodiment of the ninth aspect, one or both of Z and Z′ are —C(O)—(CR⁴₂)ₜ—R⁸.

In a fifteenth embodiment of the ninth aspect, one or both of Z and Z′ are —[U—(CR⁴₂)ₜ—NR⁵—(CR⁴₂)ₜ]ᵤ—U—(CR⁴₂)ₜ—O—(CR⁴₂)ₜ—R⁸.

In a sixteenth embodiment of the ninth aspect, one or both of Z and Z′ are —U—(CR⁴₂)ₜ—NR⁵—(CR⁴₂)ₜ—U—(CR⁴₂)ₜ—O—(CR⁴₂)ₜ—R⁸.

In a seventeenth embodiment of the ninth aspect, one or both of Z and Z′ are —C(O)—(CR⁴₂)ₜ—NR⁵—(CR⁴₂)ₜ—C(O)—(CR⁴₂)ₜ—O—(CR⁴₂)ₜ—R⁸.

In an eighteenth embodiment of the ninth aspect, one or both of Z and Z′ are —U—(CR⁴₂)ₜ—O—(CR⁴₂)ₜ—R⁸.

In a nineteenth embodiment of the ninth aspect, one or both of Z and Z′ are —C(O)—(CR⁴₂)ₜ—O—(CR⁴₂)ₜ—R⁸.

In a twentieth embodiment of the ninth aspect, one or both of Z and Z′ are —C(O)—(CR⁴₂)ₙ—NR⁷—R⁸ wherein R⁷ and R⁸ together form a 4-7 membered ring.

A tenth aspect of the invention provides a pharmaceutical composition comprising the compounds of the invention.

An eleventh aspect of the invention provides use of the compounds of the invention in the manufacture of a medicament.

In a first embodiment of the eleventh aspect the medicament is for the treatment of hepatitis C.

A twelfth aspect of the invention provides a method of treating hepatitis C comprising administering to a subject in need thereof, a therapeutically effective amount of any one of the compounds of the invention.

General Synthesis

The compounds of the invention are prepared by synthetic techniques as they are illustrated in the various synthetic schemes outlined below. In general, the synthesis started with the construction of a central core, which was followed by further elaboration of the two ends in parallel or individually. The preparation of the central biaryl system typically employs crossing coupling techniques such as Suzuki-Miyaura or Stille coupling for connecting aryl-aryl bonds.

The following abbreviations are used throughout this application:
ACN Acetonitrile
AcOH Acetic acid
aq Aqueous
Bn Benzyl
BnOH Benzyl alcohol
Boc t-butoxycarbonyl
DCE Dichloroethane
DCM Dichloromethane
DIEA(DIPEA) Diisopropylethylamine
DMA N,N-Dimethylacetamide
DME 1,2-Dimethoxyethane
DMF N,N-Dimethylformamide
DMSO Dimethylsulfoxide
DMTMM 4-(4,6-Dimethoxy-1,3,5-triazin-2-yl)-4-methylmorpholinium chloride
DPPA Diphenylphosphoryl azide
DTT Dithiothreitol
EDCI 1-Ethyl-3-[3-(dimethylamino) propyl]carbodiimide hydrochloride
EDTA Ethylene diamine tetraacetic acid
ESI Electrospray Ionization
Et₃N, TEA Triethylamine
EtOAc, EtAc Ethyl acetate
EtOH Ethanol
g Gram(s)
h Hour(s)
HBTU O-Benzotriazol-1-yl-N,N,N′,N′-tetramethyluronium hexafluorophosphate
HOBt 1-Hydroxybenzotriazole
IC₅₀ The concentration of an inhibitor that causes a 50% reduction in a measured activity
LAH Lithium aluminum hydride
LDA Lithium diisopropylamide
LCMS Liquid Chromatography Mass Spectrometry
MeI Methyl Iodide
MeOH Methanol
min Minute(s)
mmol Millimole(s)
NMM 4-Methylmorpholine
NMP N-methylpyrrolidinone
PG Protective Group
PTT Phenyl trimethyl tribromide
Py Pyridine
rt Room temperature
TEA Triethylamine
Tf Trifluoromethanesulfonate
TFA Trifluoroacetic acid
TFAA Trifluoroacetic anhydride
THF Tetrahydrofuran
TLC Thin Layer Chromatography

Reagents and solvents used below can be obtained from commercial sources such as Aldrich Chemical Co. (Milwaukee, Wis., USA). ¹HNMR spectra were recorded on a Bruker 400 MHz or 500 MHz NMR spectrometer. Significant peaks are tabulated in the order: chemical shift, multiplicity (s, singlet; d, doublet; t, triplet; q, quartet; m, multiplet; br s, broad singlet), coupling constant(s) in Hertz (Hz) and number of protons.

The following examples are provided by way of illustration only and not by way of limitation. Those of skill in the art will readily recognize a variety of noncritical parameters that could be changed or modified to yield essentially similar results. Efforts have been made to ensure accuracy with respect to numbers used (e.g., amounts, temperatures, etc.), but some experimental error and deviation should, of course, be allowed for.

LC-MS data were obtained as follows: Aglient Prep-C₁₈ Scalar, 5 μm (4.6×50 mm, flow rate 2.5 mL/min) eluting with a H₂O-MeCN gradient containing 0.1% v/v ammonia over 7 min with UV detection at 215 and 254 nm. Gradient information: 0.0-0.1 min: 95% H₂O-5% MeCN; 0.1-5.0 min; Ramp from 95% H₂O-5% MeCN to 5% H₂O-95% MeCN; 5.0-5.5 min: Hold at 5% H₂O-95% MeCN; 5.5-5.6 min: Hold at 5% H₂O-95% MeCN, flow rate increased to 3.5 mL/min; 5.6-6.6 min: Hold at 5% H₂O-95% MeCN, flow rate 3.5 mL/min; 6.6-6.75 min: Return to 95% H₂O-5% MeCN, flow rate 3.5 mL/min; 6.75-6.9 min: Hold at 95% H₂O-5% MeCN, flow rate 3.5 mL/min; 6.9-7.0 min: Hold at 95% H₂O-5% MeCN, flow rate reduced to 2.5 mL/min. Mass spectra were obtained using an electrospray ionization (ESI) source in either the positive or negative mode.

The compounds were named using ChemDraw program from Cambridge Soft Inc.

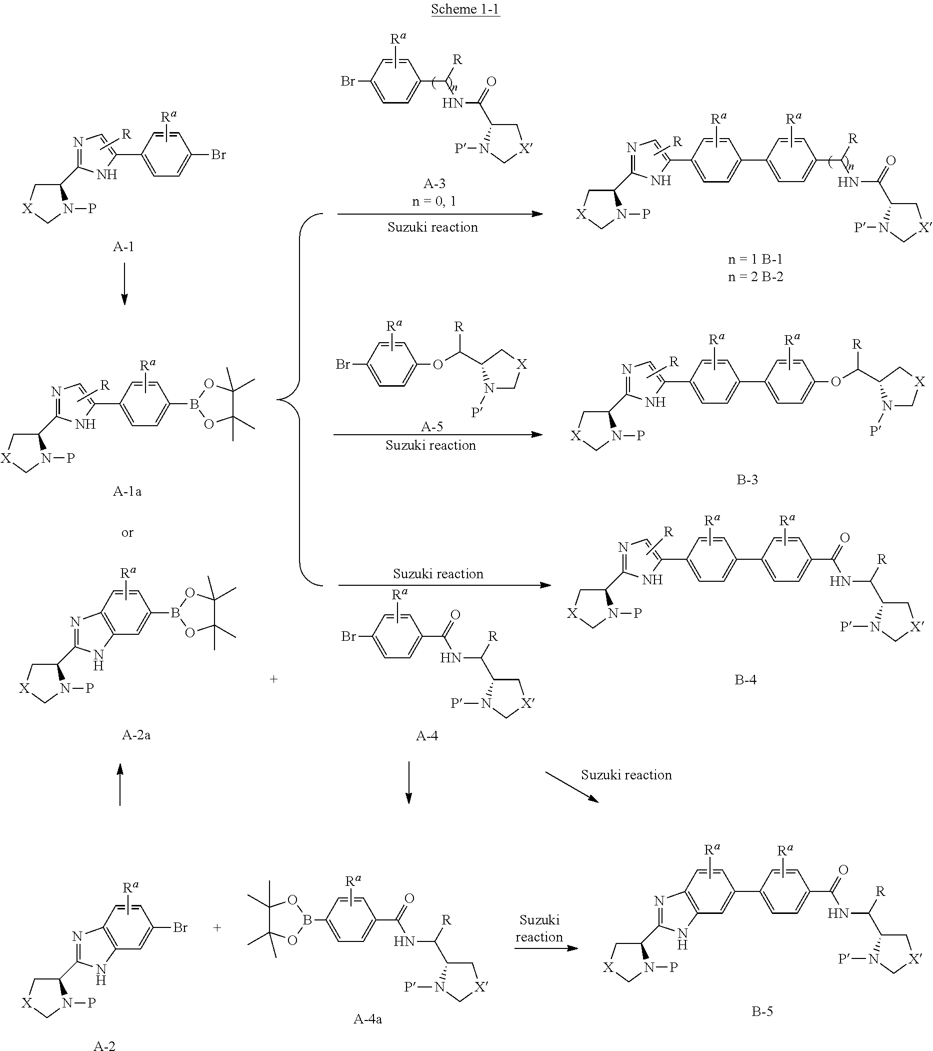

### Example 1

### Preparation of Biphenyl Core Structures

Scheme 1-1 depicts the general synthesis of a number of representative core structures that contain a biaryl unit. For illustrative purposes, a substituted phenyl ring is used to represent an aryl group. The phenylimidazole intermediate A-1, prepared by modifying reported procedures and detailed later, is converted to its corresponding borate by treatment with a diborane agent such as 4,4,4′,4′,5,5,5′,5′-octamethyl-2,2′-bi(1,3,2-dioxaborolane) in the presence of a palladium catalyst, typically Pd(dppf)Cl₂, and a base such as triethylamine to give the arylborate intermediate A-1a (borates, A-2a, A-4a and others can be prepared similarly and used in similar fashion as A-1a in the following step). Under the similar cross coupling conditions (Suzuki reaction), compound A-1a (or A-2a or A-4-a) reacts with an aryl bromide or iodide such as A-3, A-4 or A-5 to give the respective cross-coupled product B-1, B-2, B-3 or B-4. Using the preparation of B-5 as an example, the scheme is intended to show that such a cross coupling can be achieved in multiple ways. The coupling of A-2a and A-4 will lead to the biaryl compound B-5. Alternatively, reacting A-2 and A-4-a can also afford B-5.

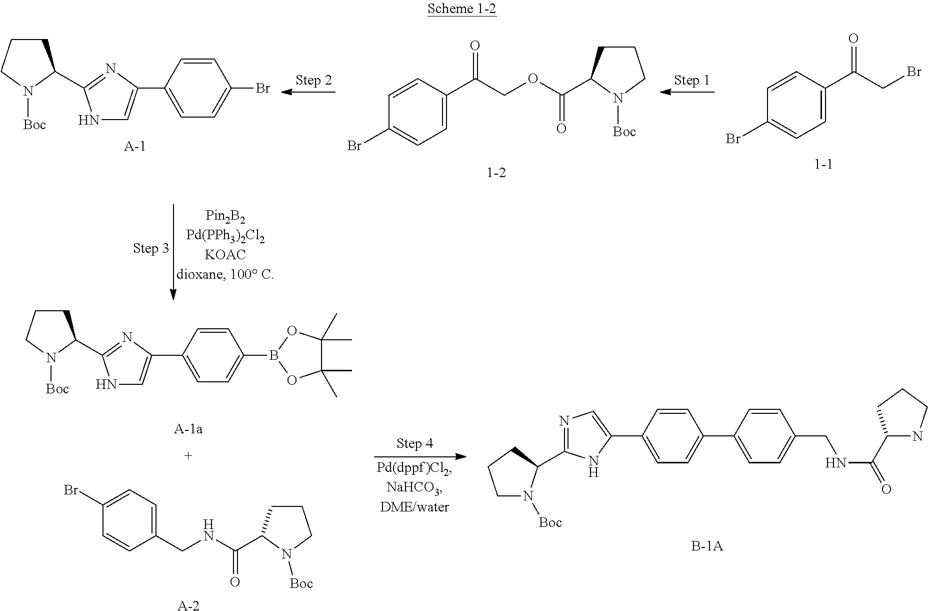

Scheme 1-2 and the procedures described below details a typical method of preparing a biphenyl structure employing the Suzuki coupling reaction.

Step 1. (S)—N-Boc-Pro-OH (97.0 g, 0.45 mol) and Et₃N (130 g, 1.29 mol) were added to a solution of 2-bromo-1-(4-bromophenyl)ethanone 1-1 (120 g, 0.43 mol) in CH₃CN (300 mL). After stirring at rt for 2 h, the mixture was concentrated under reduced pressure to afford (S)-2-(2-(4-bromophenyl)-2-oxoethyl) 1-tert-butyl pyrrolidine-1,2-dicarboxylate, 1-2. The crude product was used for next step without further purification.

Step 2. NH₄OAc (300 g, 3.90 mol) was added to a solution of (S)-2-(2-(4-bromophenyl)-2-oxoethyl) 1-tert-butyl pyrrolidine-1,2-dicarboxylate (159 g, 0.39 mol) in xylene (250 mL). The mixture was stirred at 140° C. overnight. The mixture was concentrated under reduced pressure, the residue was purified by silica gel column chromatography (10:1 petroleum ether/EtOAc) to afford (S)-tert-butyl 2-(4-(4-bromophenyl)-1H-imidazol-2-yl)pyrrolidine-1-carboxylate, A-1, (105 g, 70%) as a white solid: ¹H NMR (500 MHz, CDCl₃) δ 1.48 (s, 9H), 1.96 (m, 1H), 2.16 (m, 2H), 3.01 (m, 1H), 3.42 (m, 2H), 4.96 (d, 1H, J=5.5 Hz), 7.22 (s, 1H), 7.46-7.55 (m, 4H) ppm; LC-MS (ESI): m/z 392.1 (M+H)⁺.

Step 3. Pd(dppf)Cl₂ (400 mg, 0.500 mmol) was added to a mixture of A-1 (4.90 g, 12.5 mmol), bis(pinacolato)diboron (7.10 g, 26.3 mmol), potassium acetate (3.20 g, 32.5 mmol) in 1,4-dioxane (100 mL). After stirring at 80° C. for 3 h, the reaction mixture was filtered and concentrated in vacuo. The residue was purified with silica gel column chromatography (2:1 PE/EA) to provide A-1a (3.0 g, 53%) as a gray solid: LCMS (ESI): m/z 440 (M+H)⁺.

Step 4. A sample of Pd (dppf)Cl₂ (0.270 g, 0.368 mmol) was added to a mixture of (S)-tert-butyl 2-(4-bromobenzylcarbamoyl)pyrrolidine-1-carboxylate A-2 (3.53 g, 9.21 mmol), the aryl 4,4,5,5-tetramethyl-1,3,2-dioxaborolane A-1a (4.05 g, 9.21 mmol) and NaHCO₃ (2.63 g, 31.3 mmol) in DME (78 mL) and water (26 mL). The reaction mixture was heated at 80° C. for 6 h then allowed to cool to rt. Water (100 mL) was added and the product was extracted with 20% MeOH/CHCl₃ (2×100 mL). The organic layers were combined, washed with brine and the solvent was removed in vacuo. The crude product was purified by column chromatography (50% EtOAc/DCM to 100% EtOAc), to give. (S)-tert-butyl 2-((4′-(2-((S)-1-(tert-butoxycarbonyl)pyrrolidin-2-yl)-1H-imidazol-5-yl)biphenyl-4-y)methylcarbamoyl)pyrrolidine-1-carboxylate. B-1A (4.62 g, 81% yield). ¹HNMR (DMSO-d₆, 400 MHz): δ 12.21-12.16/11.95-11.75 (m, 1H), 8.46-8.32 (m, 1H), 7.86-7.22 (m, 9H), 4.90-4.70 (m, 1H), 4.42-4.03 (m, 3H), 3.59-3.22 (m, 4H), 2.30-1.68 (m, 8H), 1.48-1.01 (m, 18H) ppm. LC-MS (ESI): m/z 616 (M+H)⁺.

The following biaryl cores, B-1B, B-1C, B-1D, B-1E, B-1F, and B-2A, B-2B, B-2C, B2D, B2E and B-2F were prepared similarly.

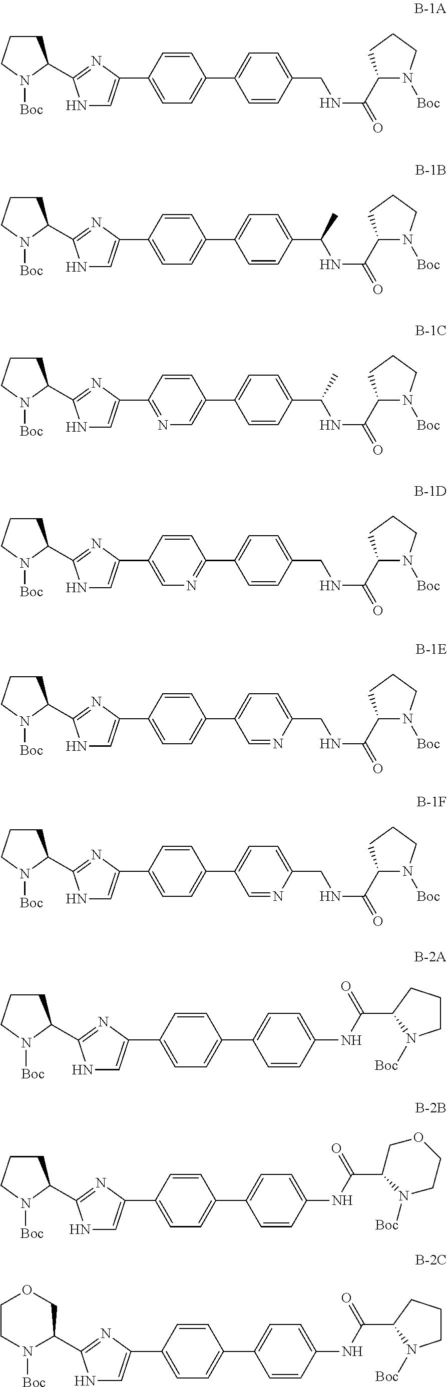

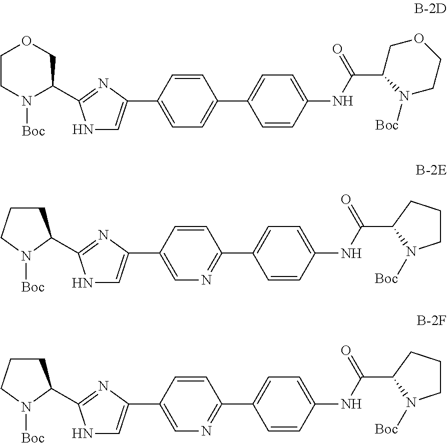

B-2A: ¹H NMR (500 MHz, CDCl₃) δ 9.26 (s, 1H), 7.70-7.74 (m, 3H), 7.55-7.59 (m, 5H), 7.36-7.46 (m, 10H), 7.24-7.26 (m, 1H), 5.66 (m 1H), 5.62 (d, J=7.0 Hz, 1H), 5.41 (d, J=7.0 Hz, 1H), 5.36 (m, 2H), 4.80 (d, J=7.5 Hz), 3.8-3.84 (m, 2H), 3.23 (m, 2H), 2.88 (m, 1H), 2.53 (m, 1H), 1.62-2.09 (m, 6H), 1.41-1.42 (m, 18H) ppm. LC-MS (ESI) (m/z): 602 (M+1)⁺.

B-2B: LC-MS (ESI): m/z: 618 (M+1)⁺.

B-2C: LC-MS (ESI): m/z 618 (M+1)⁺.

B-2D: LC-MS (ESI): m/z 634 (M+1)⁺.

B-2E: LC-MS (ESI): m/z 601 (M−1)⁺.

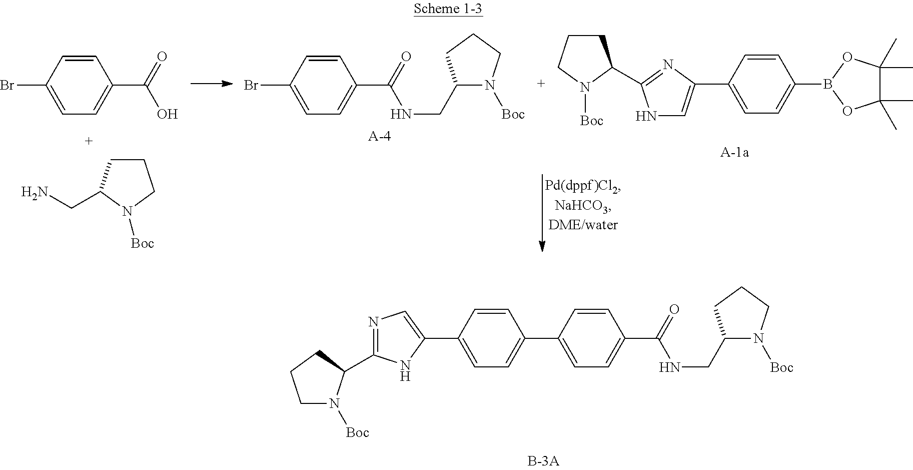

B-3A was prepared according to Scheme 1-3. LC-MS (ESI): m/z 616 (M+H)⁺, >95% purity. ¹H NMR (DMSO-d₆, 400 MHz): δ 12.01-11.55/11.75-11.55 (m, 1H), 8.37-8.25 (m, 1H), 7.74-7.23 (m, 9H), 4.65-4.48 (m, 1H), 3.75-3.61 (m, 1H), 3.38-2.94 (m, 6H), 1.89-1.44 (m, 8H), 1.48-1.01 (m, 18H) ppm.

B-3B, B-3C, B3-D, B-3E, and B-3F were prepared similarly to B-3A.

B-3B: LC-MS (ESI): m/z 617 (M+1)⁺.

B-3C: LC-MS (ESI): m/z 615 (M−1)⁺.

B-3D: LC-MS (ESI): m/z 591 (M+1)⁺.

B-3E: LC-MS (ESI): m/z 590 (M+1)⁺.

B-3F: LC-MS (ESI): m/z 591 (M+1)⁺.

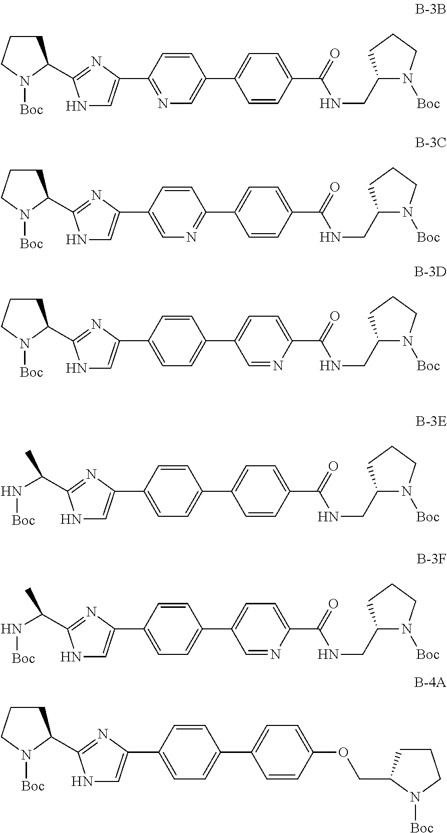

Compound B-4A is prepared by following the procedures described in the synthesis of B-1A and substituting (S)-tert-butyl 2-(4-bromobenzylcarbamoyl)pyrrolidine-1-carboxylate (A-3) with (S)-tert-butyl 2-((4-bromophenoxy)methyl)pyrrolidine-1-carboxylate (A-5). B-4A: LC-MS (ESI): m/z 589 (M+H)⁺, 90% purity. ¹H NMR (DMSO-d₆, 400 MHz, 373K): δ 11.57-11.42/11.30-11.10 (m, 1H), 7.59-6.92 (m, 9H), 4.64-4.57 (m, 1H), 3.94-3.87 (m, 1H), 3.78-3.72 (m, 1H), 3.34-3.27 (m, 1H), 3.22-3.00 (m, 3H), 2.02-1.50 (m, 8H), 1.24-0.82 (m, 18H) ppm.

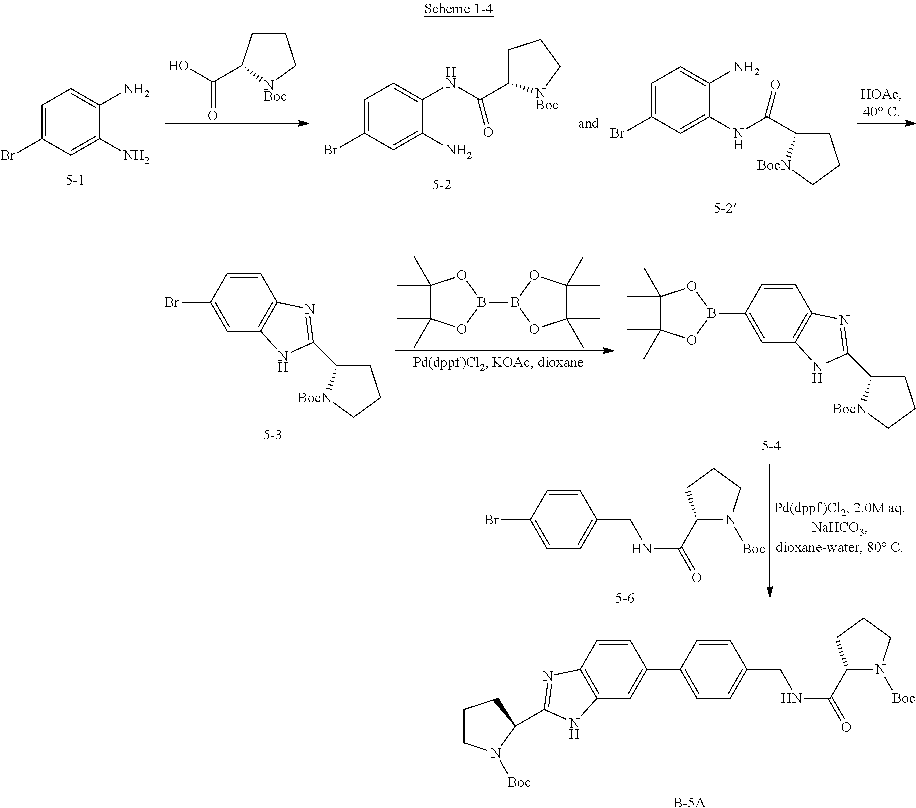

Step 1. Referring to Scheme 1-4, HATU (51 g, 135 mmol) was added to a solution of N-Boc-L-Pro-OH (29 g, 135 mmol) and DIPEA (29 g, 225 mmol) in THF (500 mL) rt. After stirring at rt for 10 min, 4-bromobenzene-1,2-diamine 5-1 (25 g, 135 mmol) was added. After stirring at rt for several hours, the reaction mixture was concentrated and the residue was diluted with EtOAc (500 mL). The resulting mixture was washed with water for several times (100 mL×3) and dried with anhydrous Na₂SO₄. The solvent was removed and the residue was dried in vacuo to give a mixture of crude compounds 5-2 and 5-2′, which were used for the next step without further purification. LC-MS (ESI): m/z 384.1 (M+H)⁺.

Step 2. A mixture of crude compounds 5-2 and 5-2′ in AcOH (1000 mL) was stirred at 40° C. for 12 h. Subsequently, the reaction mixture was carefully neutralized by adding saturated aqueous sodium bicarbonate solution to adjust to pH 8. The resulting mixture was extracted with EtOAc for several times (250 mL×3). The extracts were combined, washed with water, and dried with anhydrous Na₂SO₄. The solvent was removed and the residue was purified by silica gel chromatography (Petroleum ether/EtOAc=4/1 (v/v)) to give compound 5-3 (35 g, 71% yield) as a yellow solid. LC-MS (ESI): m/z 366.1 (M+H)⁺.

Step 3. Pd(dppf)Cl₂ (680 mg, 0.7 mmol) was added to a mixture of compound 5-3 (5.0 g, 13.7 mmol), bis(pinacolato)diboron (10.4 g, 41.1 mmol), potassium acetate (4.0 g, 41.1 mmol) in 1,4-dioxane (100 mL) at rt under an atmosphere of N₂. After stirring at 80° C. for 3 h under an atmosphere of N₂, the reaction mixture was filtered through CELITE™ 545 and the filter cake was washed with EtOAc for several times (50 mL×3). The filtrate was washed with brine and dried with anhydrous Na₂SO₄. The solvent was removed and the residue was purified by silica gel column chromatography (Petroleum ether/EtOAc=2/1 (v/v)) to give compound 5-4 (3.3 g, 58% yield). LC-MS (ESI): m/z 414.2 (M+H)⁺.

Step 4. A mixture of (S)-tert-butyl 2-(4-bromobenzylcarbamoyl)pyrrolidine-1-carboxylate 5-6 (1.54 g, 4.0 mmol), (S)-tert-butyl 2-(6-(4,4,5,5-tetramethyl-1,3,2-dioxaborolan-2-yl)-1H-benzo[d]imidazol-2-yl)pyrrolidine-1-carboxylate 5-4 (1.65 g, 4.0 mmol), Pd(dppf)Cl₂ (163 mg, 0.2 mmol), and Na₂CO₃ (1.44 g, 13.6 mmol) in a mixture of dioxane and water (30.0 mL/6.0 mL) was purged with nitrogen. The resulting mixture was heated at 95° C. for 7.5 h, and then all solvent was removed to give a crude mixture. The crude mixture was diluted with dichloromethane (100 mL), which was washed twice with water and brine, dried over Na₂SO₄, filtered, and concentrated. The crude mixture was purified by column chromatography eluting with EtOAc only to yield B-5A as a yellow solid (2.13 g, 90%). LC-MS (ESI): m/z 490.3 (M+H)⁺.

Compounds B-7B, B-7C, B-7D, B-7E, B-7F were obtained by reacting (5)-tert-butyl 2-(6-(4,4,5,5-tetramethyl-1,3,2-dioxaborolan-2-yl)-1H-benzo[d]imidazol-2-yl)pyrrolidine-1-carboxylate A-2a and the respective phenyl bromide coupling counter parts under similar conditions described in Step 4 above.

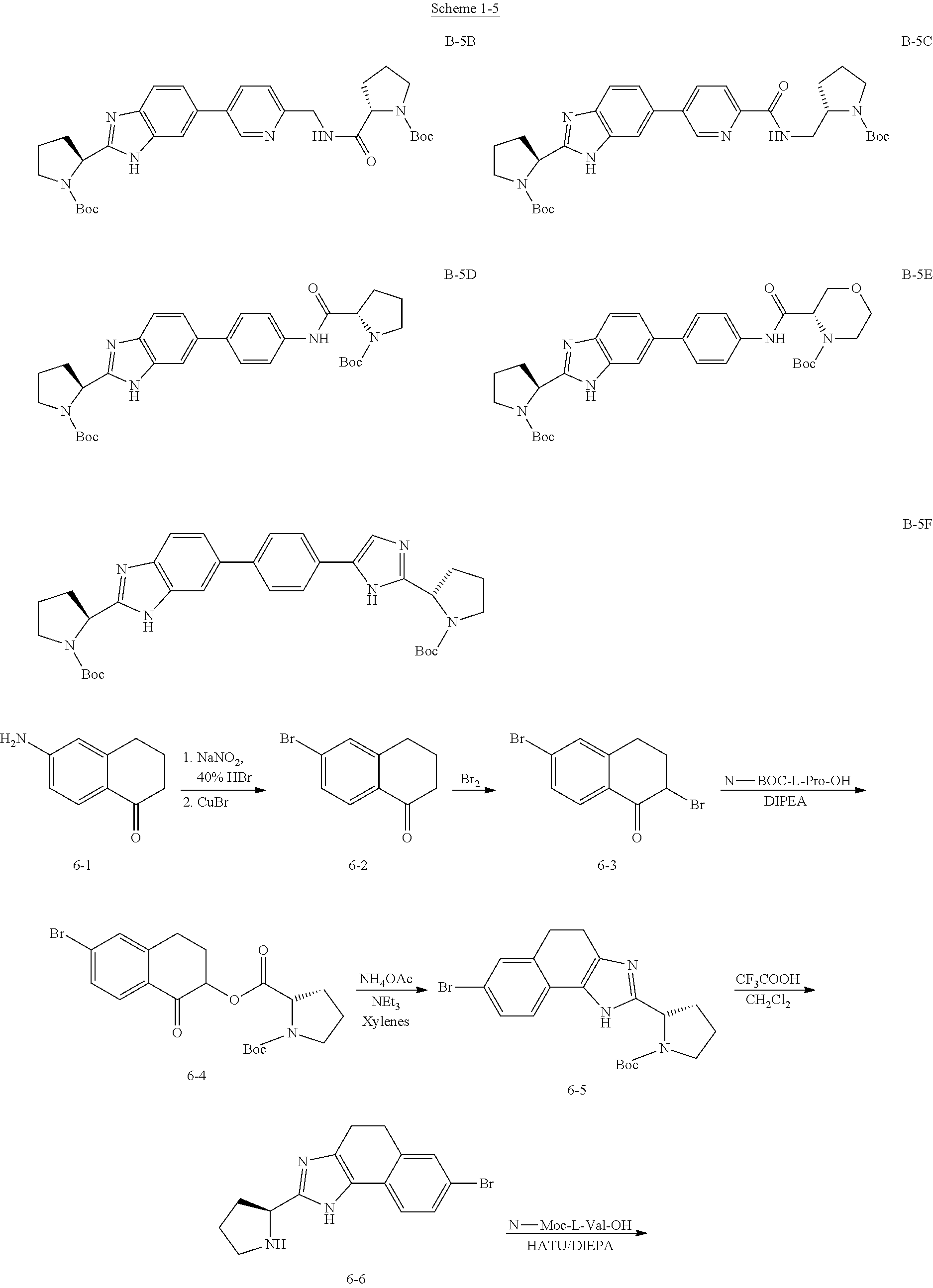

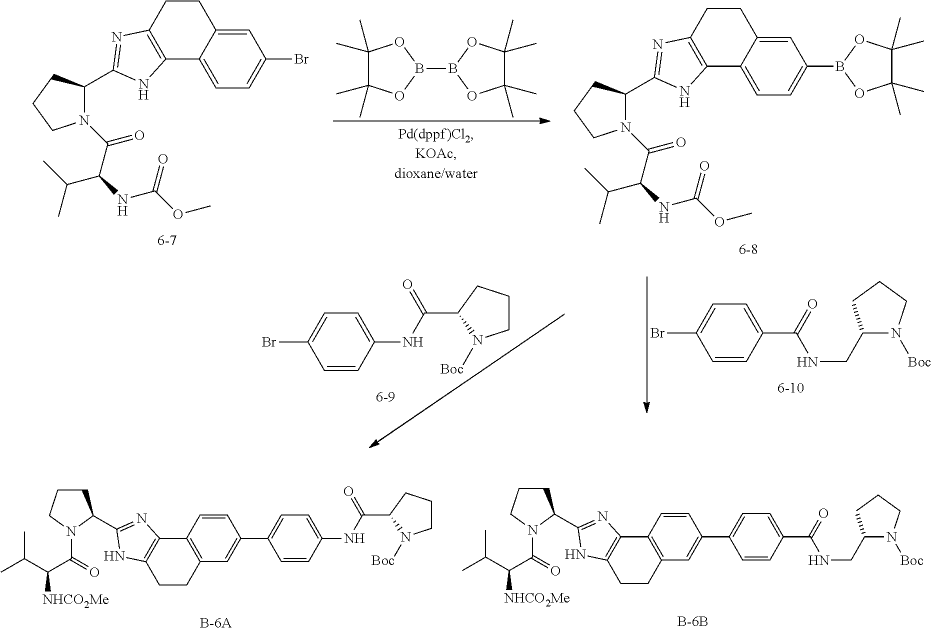

Scheme 1-5 and the conditions below were utilized in the preparation of core structures bearing a benzoimidazole moiety using conditions for the synthesis of B-6A and B-6B.

Step 1. A solution of 9.72 g (0.141 mol) of sodium nitrite in 18 mL of water was added to a solution of 6-1 (20.60 g, 0.128 mol) in 45 mL of 48% hydrobromic acid and 10 mL of water, maintaining a temperature below 5° C. After stirring at 5° C. for 1 h, CuBr (0.128 mol) was added and the resulting mixture was stirred at rt for 3 h. Subsequently, the mixture was extracted with EtOAc (2×200 mL). The extracts were combined, washed with brine, and dried with anhydrous Na₂SO₄. The solvent was removed and the residue was purified by silica gel column chromatography (Hexane/EtOAc=12/1 (v/v)) to afford 6-2 (13.3 g, 46% yield) as a powder. ¹H NMR (CDCl₃, 400 MHz) δ 7.90 (d, 1H), 7.44 (m, 2H), 2.96 (t, 2H), 2.64 (t, 2H), 2.15 (m, 2H) ppm.

Step 2. 3.1 mL of bromine was slowly added to a solution of 6-2 (12.49 g, 55.5 mmol) in methylene chloride (300 mL) and 0.30 mL of 48% hydrobromic acid at 0° C. The reaction mixture was gradually warmed up to rt, and kept stirring for another 2 h. The organic solution was washed with saturated NaHCO₃ twice, and then with water. The crude product was purified by silica gel column chromatography to afford 6-3 (11.9 g, 71% yield). ¹H NMR (CDCl₃, 400 MHz) δ 7.94 (d, 2H), 7.52 (m, 2H), 4.72 (t, 1H), 3.32 (m, 1H), 2.92 (m, 1H), 2.48 (m, 2H) ppm.

Step 3. A mixture of 6-3 (11.80 g, 38.8 mmol), N-Boc-L-Pro-OH (10.02 g, 46.6 mmol), and diisopropylethylamine (7.02 g, 54.3 mmol) in acetonitrile (200 mL) was stirred at 50° C. for 10 h. The solvent was evaporated and the residue was partitioned between methylene chloride and water. The organic layer was separated and concentrated to dryness. The crude product was purified by silica gel column chromatography (hexanes/ethyl acetate=1/7 to 1/4 (v/v)) to provide 6-4 (11.53 g, 68% yield) as a white solid. ¹H NMR (CDCl₃, 400 MHz) δ 7.84 (m, 1H), 7.48 (m, 2H), 5.58 (m, 1H), 4.40 (m, 1H), 3.60 (m, 1H), 3.40 (m, 1H), 3.18 (m, 1H), 3.04 (m, 1H), 2.37 (m, 2H), 2.04 (m, 1H), 1.96 (m, 1H), 1.46 (ds, 9H) ppm.

Step 4. A mixture of 6-4 (11.09 g, 25.3 mmol), ammonium acetate (29.25 g, 38.0 mmol) and triethylamine (38.45 g, 38.0 mmol) in xylenes (600 mL) in a sealed tube was stirred at 140° C. for 2 h. After being cooled, the reaction mixture was transferred into a flask and concentrated to dryness. The residue was partitioned between chloroform and water, and the organic layer was washed with water, and concentrated. The crude product was purified by silica gel column chromatography (NH₄OH/acetonitrile/ethyl acetate: 1/8/100=(v/v/v)) to afford 6-5 (8.22 g, 75% yield) as a white solid. LC-MS (ESI): m/z 420.1 (M+H)⁺.

Step 5. Trifluoroacetic acid (20 mL) was slowly added into a solution of 8-5 (4.80 g, 11.4 mmol) in methylene chloride (40 mL) at rt. After stirring at rt for 2 h, the reaction mixture was concentrated and the residue was dried in vacuo to give a TFA salt 6-6, which was used for the next step without further purification. LC-MS (ESI): m/z 318.1 (M+H)⁺.

Step 6. DIPEA (22.8 mL, 138 mmol) was added to a mixture of the TFA salt 6-6 (6.28 g, 11.5 mmol) in DMF (23 mL), followed by N-Moc-L-Val-OH (2.42 g, 13.8 mmol) and HATU (5.25 g, 13.8 mmol). After stirring at rt for 2 h, the reaction mixture was slowly dropped into water while stirring. The resulting precipitate was collected by filtration. The crude product was purified by silica gel column chromatography (Hexane/Ethyl Acetate=1/4 to 0/1 (v/v)) to afford 6-7 (4.43 g, 81% yield). LC-MS (ESI): m/z 475.3 (M+H)⁺.

Step 7. To a mixture of compound 6-7 (2.5 g, 5.27 mmol), bis(pinacolato)diboron (2.6 g, 10.5 mmol), potassium acetate (2.2 g, 15.8 mmol) in 1,4-dioxane (50 mL) was added Pd(dppf)Cl₂ (260 mg, 0.3 mmol) at rt under an atmosphere of N₂. After stirring at 80° C. for 3 h under an atmosphere of N₂, the reaction mixture was filtered through CELITE™ 545 and the filter cake was washed with three 30 mL aliquots of EtOAc. The filtrate was washed with brine and dried with anhydrous Na₂SO₄. The solvent was removed and the residue was purified by silica gel column chromatography (Petroleum ether/EtOAc=2/1 (v/v)) to give compound 6-8 (1.6 g, 58% yield). LC-MS (ESI): m/z 522.3 (M+H)⁺.

Step 8. Compounds B-6A and B-6B were obtained by reacting borate 6-8 with the respective bromide 6-9 and 6-10 under similar Suzuki cross coupling conditions described.

### Example 2

### Preparation of Aryl Ether Core Structures

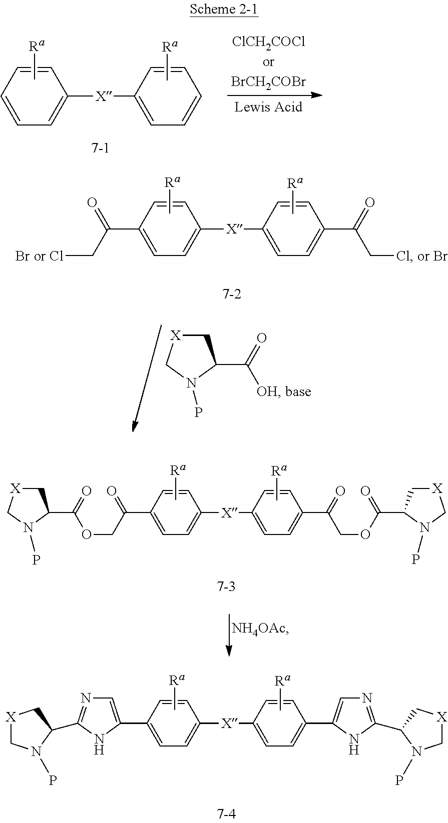

Scheme 2-1 illustrates one of the ways to prepare molecules containing an arylether, thioarylether moiety as the central scaffold. The R^{a}'s are each independently present or absent. The synthesis starts with a Friedel-Craft acylation reaction between a biaryleather or thiobiaryl ether compound 7-1 with chloroacetyl chloride (or bromoacetyl bromide to obtain the corresponding dibromide). Alkylation of the resulting bischloroacetylphenone, 7-2, with N-protected L-proline to give the bisprolinyl ester 7-3. When such a bis ester is treated with an excess amount (10 equivalents) of ammonium acetate in toluene or xylenes under heating, the bisimidazole compound 7-4 is formed. Those skilled in the art will know that other means to assemble such a structure do exist, including the formation of an amide equivalent of intermediate 7-3 prior to the imidazole ring formation, or the introduction of the imidazole moiety via a cross coupling operation between a suitably functionalized imidazole and a phenyl group by techniques such as Suzuki or Stille coupling.

Step 1. Several portions of AlCl₃ (47 g, 352.5 mmol) were added to a stirred solution of 7-1 (20 g, 117.5 mmol, X═CH₂, X″═O) in 250 mL DCM at 0° C. The mixture was stirred for half an hour. 2-Chloroacetyl chloride was added dropwise and the mixture was then removed to rt and stirred for another 2 h. After completion of the reaction, the reaction mixture was then poured into ice water (200 mL) under violent stirring, and extracted with EtOAc (200 mL×2). The organic layer was washed with water (50 mL×2) and then dried over Na₂SO₄. The solvent was removed and the residue was purified by silica gel column chromatography (PE:EA=6:1) to give 7-2 (26 g, 68%) as a yellow solid. LC-MS (ESI): m/z 322.9 (M+H)⁺, 344.9 (M+Na)³⁰ .

Step 2. A solution of 7-2 (10 g, 30.94 mmol) in 20 mL MeCN was added to a stirred solution of N-(tert-butoxycarbonyl)-L-proline (16.65 g, 77.35 mmol) in 200 mL MeCN followed by addition of Et₃N (12.53 g, 123.76 mmol). The mixture was stirred at rt overnight. The solvent was removed and the residue was purified by silica gel column chromatography (PE:EtOAc=3:1) to give 7-3 (13.05 g, 63.4%) as a white solid. ¹H NMR (500 MHz, CDCl₃) δ 1.42 (d, 18H), 1.86 (m, 2H), 2.02-2.14 (m, 3H), 2.24-2.36 (m, 4H), 3.41-3.62 (m, 4H), 4.39-4.49 (m, 2H), 5.18-5.58 (m, 4H), 7.06-7.14 (m, 4H), 7.95-8.01 (m, 4H) ppm; LC-MS: m/z 681.0 (M+H)⁺.

Step 3. A solution of 7-3 (10 g, 14.69 mmol) in 100 mL toluene was added NH₄OAc (22.65 g, 293.82 mmol). The mixture was heated to reflux and stirred at this temperature overnight. The next morning the reaction mixture was cooled to rt and washed with saturated NaHCO₃ until pH value equaled about 8, the organic phase was separated, dried over Na₂SO₄, concentrated and purified by silica gel column chromatography (PE:EA=50:1 to 2:1) to obtain 7-4 (2.6 g, 28%). LCMS: Anal. Calcd. for C₃₆H₄₄N₆O₅ 640.34. Found 641.1 (M+H)⁺.

Following the procedures description above and substituting diphenyl ether with thiophenyl ether in Step 1, the thioether analog of 7-4 (X″═S) was obtained.

### Example 3

### Preparation Biphenyl Analogs

Once the core scaffolds are built, they can be further converted to analogs intended for enhancing antiviral potency and physicochemical properties, primarily through the further functionalization of the terminal amino groups (pyrrolidines as in these examples shown).

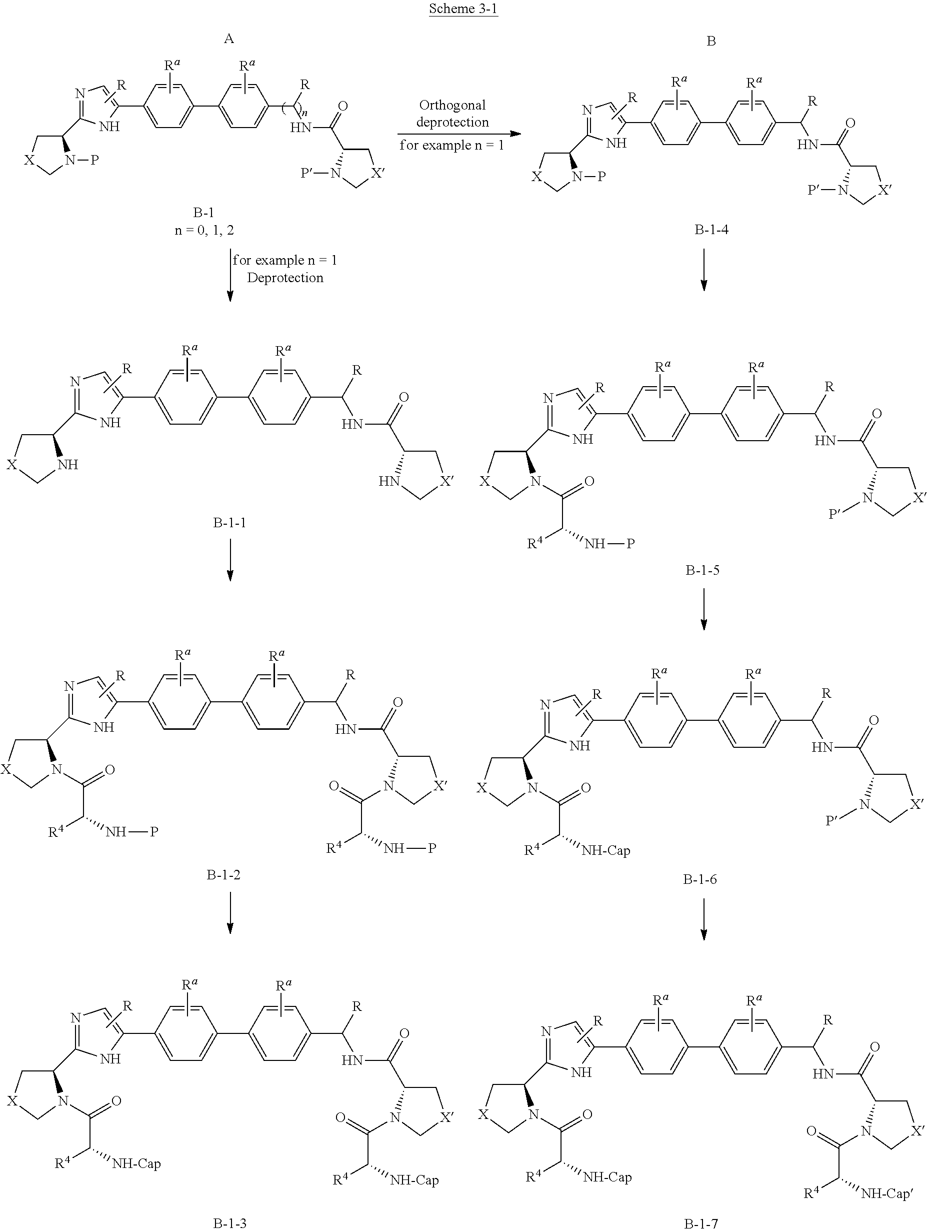

Scheme 3-1 illustrates two major routes (A and B) for further functionalizing the central scaffold. R₂ and R₃ in Scheme 3-1 are defined as R^{a} in formula I. R₁ and R₄ in Scheme 3-1 are defined as R in formula I. R in Scheme 3-1 is defined as R⁵ in formula I. In route A, where the nitrogen protecting groups, P and P′, are introduced to be the same or both are unmasked at the first step (B-1 to B-1-1), both ends of the molecule can undergo further transformations in parallel fashion. In Route B, the orthogonally protected nitrogen atoms of the pyrrolidines are unmasked selectively and the two ends of the molecules are functionalized individually, allowing for the introduction of different amino acid residues and the capping groups.

Starting from a properly protected B-1, the nitrogen protecting groups P and P′ can be removed simultaneously to give free diamines B-1-1. When B-1-1 is treated together with a properly protected amino acid under standard peptide coupling conditions, such as the combination of HATU and Hünig's base, the doubly coupled product B-1-2 is obtained. When P is one of the removable protecting groups, it is removed to free the amino group for further derivatization to B-1-3. The definition of Cap and Cap′ group is described previously. Selective removal of P over P′ will lead to B-1-4. Those skilled in the art will understand that the P′ group can generally be deprotected while the P group is preserved to give an alternative form of B-1-4 like structure. The free amino group of B-1-4 is coupled with another properly functionalized amino acid to give B-1-5. When this process of selective deprotection and functionalization is repeated, compound B-1-6 is obtained. The newly introduced amino acid in B-1-6 can be the same as the residue on the left-hand side of the molecule and can be a different one. From B-1-6, a variety of compounds (with a general formula of B-1-7) are prepared with differentially functionalized end pieces.

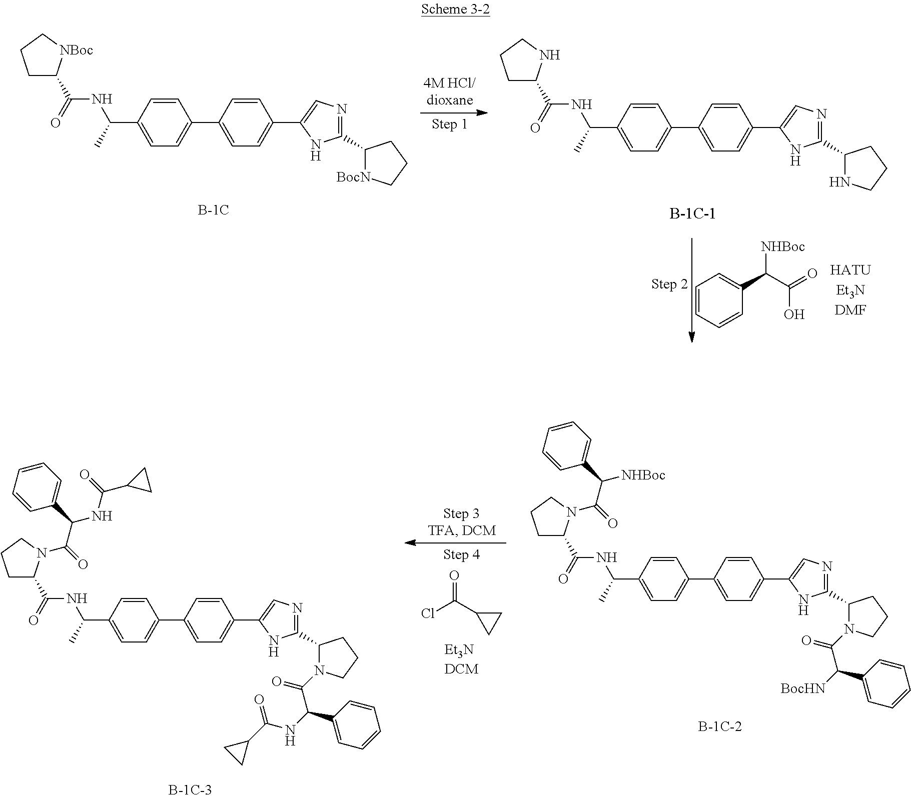

Scheme 3-2 illustrates further functionalization of core intermediates.

Step 1. 4 N HCl in dioxane (1.667 mL, 6.67 mmol) was added to a stirred solution of (S)-tert-butyl 2-((S)-1-(4′-(2-((S)-1-(tert-butoxycarbonyl)pyrrolidin-2-yl)-1H-imidazol-5-yl)biphenyl-4-yl)ethylcarbamoyl)pyrrolidine-1-carboxylate (1 g, 1.588 mmol) (B-1C) in dioxane (12 mL). After stirring at rt for 4 h, additional 4.0 N HCl in dioxane (0.85 mL) was added and the reaction stirred at rt for an additional 18 h. The solvents were removed in vacuo to give the desired compound (B-1C-1) which was used as is in subsequent steps.

Step 2. DIPEA (3.19 mL, 18.30 mmol) was added to a stirred solution of (S)—N—((S)-1-(4′-(2-((S)-pyrrolidin-2-yl)-1H-imidazol-5-yl)biphenyl-4-yl)ethylpyrrolidine-2-carboxamide (0.914 g, 2.128 mmol), N-Boc-D-phenylglycine-OH (1.176 g, 4.68 mmol) and HATU (1.699 g, 4.47 mmol) in 40 mL DMF at 0° C. After stirring at 0° C. for 45 min, water (150 mL) was added, followed by EtOAc (150 mL) and the layers were separated. The aqueous layer was washed with EtOAc (150 mL). The combined organic layers were washed with water (2×200 mL), brine (2×200 mL), dried over MgSO₄, filtered and concentrated in vacuo. The crude product was purified by column chromatography (10% EtOAc/DCM). Further purifications by SCX and column chromatography (0-5% MeOH/DCM) gave B-1C-2. ¹H NMR (500 MHz, d⁶-DMSO) δ 11.63 and 11.78-11.84 (m, m, 1H), 8.07-8.12 (m, 1H), 7.74-7.77 (m, 2H), 7.24-7.57 (m, 13H), 7.07 and 7.13 (m, m, 2H), 5.34-5.38 (m, 2H), 4.87-5.01 (m, 2H), 4.23-4.26 (m, 1H), 3.78-3.90 (m, 2H), 2.95-3.01 (m, 2H), 1.65-1.98 (m, 10H), 1.25-1.35 (m, 21H) ppm. LC-MS (ESI): m/z 896.7 (M+1)⁺.

Step 3. A solution of di-tert butoxycarbonyl (S)-1-((R)-2-amino-2-phenylacetyl)-N—((S)-1-(4′-(2-4S)-1-((R)-2-amino-2-phenylacetyl)pyrrolidin-2-yl)-1H-imidazol-5-yl)biphenyl-4-yl)ethyl)pyrrolidine-2-carboxamide (300 mg, 0.335 mmol) in 25% TFA/DCM (6 mL) was stirred at rt for 18 h. The solvents were removed in vacuo and the crude product was purified by SCX to give the free amino compound (182 mg, 0.262 mmol, 78% yield). LC-MS (ESI): m/z 696.3 (M+H)⁺, ˜95% purity @ 254 nm.

Step 4. Cyclopropane carbonylchloride (12.39 μL, 0.142 mmol) was added to a stirred solution of triethylamine (22.72 μL, 0.162 mmol) and the product from Step 3, (5)-1-((R)-2-(cyclopropanecarboxamido)-2-amino-2-phenylacetyl)pyrrolidin-2-yl)-1H-imidazol-5-yl)biphenyl-4-yl)ethyl)pyrrolidine-2-carboxamide (45 mg, 0.065 mmol) in DCM (2 mL), the mixture stirred at rt for 18 h. Water (3 mL) was added and the phases were separated using an ISP Phase separator cartridge. Evaporation of the solvent gave a yellow foam which was purified by SCX followed by column chromatography (1%-2.5% MeOH/DCM) Further purification by column chromatography (0-5% MeOH/DCM) gave B-1C-3 (28 mg, 0.034 mmol, 52.0% yield) as a white solid. B-1C-3: ¹H NMR (500 MHz, d⁶-DMSO) δ 11.93 and 11.62-11.67 (m, 1H), 8.78-8.92 (m, 2H), 8.06 (d, J=8.0 Hz, 1H), 7.77-7.83 (m, 2H), 7.30-7.68 (m, 15H), 6.94-7.12 (m, 2H), 5.73-5.75, and 5.63-5.67 (m, 2H), 5.07-5.09 (m, 1H), 4.90-4.97 (m, 1H), 4.30-4.33 (m, 1H), 3.88-3.93 (m, 2H), 3.16-3.30 (m, 2H), 1.78-2.05 (m, 10H), 1.42 (d, J=6.8 Hz, 6H), 0.52-0.72 (m, 8H) ppm. LCMS (ESI): (m/z) 833 (M+1)⁺ 98% purity.

Following the steps described above and by substituting the bis-N-Boc protected pyrrolidine building block B-1C with the amide building block B-1B, the following analogs were prepared.

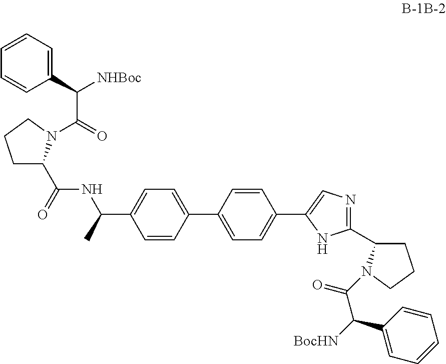

B-1B-2: LC-MS (ESI): m/z 896.3 (M+1)⁺.

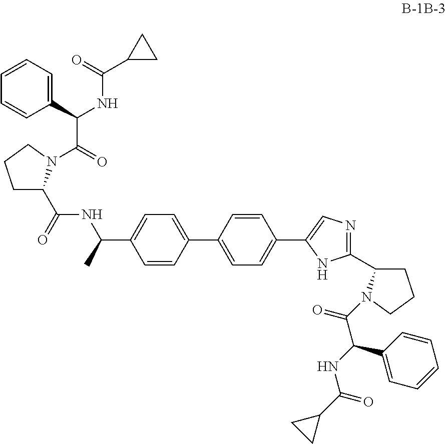

B-1B-3: ¹H NMR (500 MHz, d⁶-DMSO) δ 11.46 (m, 1H), 8.35-8.36 (m, 2H), 7.30-7.78 (m, 20H), 5.65-5.73 (m, 2H), 4.99 ans 5.17 (m, m, 2H), 4.42 (m, 1H), 3.79 (m, 2H), 3.32 (m, 2H), 2.94-3.00 (m, 2H), 1.77-1.98 (m, 10H), 1.39-1.40 (m, 3H), 0.63-0.74 (m, 8H) ppm. LCMS (ESI): m/z 832.4 (M+1)⁺.

Following the steps described above and by substituting the bis-N-Boc protected pyrrolidine building block B-1C with the amide building block B-1A, the following analogs were prepared.

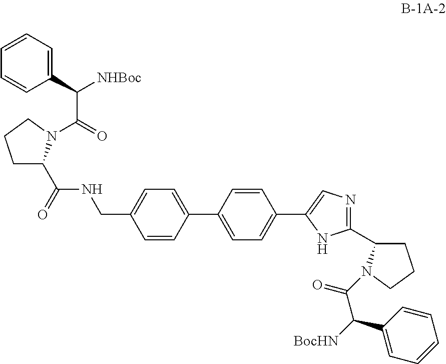

B-1A-2: LC-MS (ESI): m/z 882.5 (M+1)⁺.

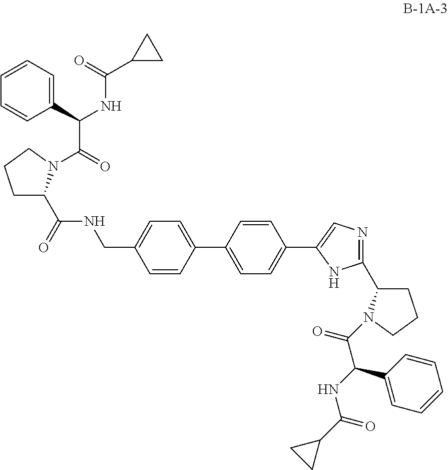

B-1A-3: ¹H NMR (500 MHz, CDCl₃) δ 7.79 (m, 2H), 7.20-7.60 (m, 17H), 6.91-6.93 and 6.68-6.69 (m, m, 2H), 5.59-5.61 (m, 2H), 5.33-5.35 (m, 1H), 4.41-4.67 (m, 3H), 3.80-3.84 (m, 2H), 3.20-3.30 (m, 2H), 2.80-2.87 (m, 1H), 2.37-2.39 (m, 1H), 1.82-2.20 (m, 6H), 1.25-1.35 (m, 2H), 0.90-1.04 (m, 2H), 0.68-0.95 (m, 6H) ppm. LC-MS (ESI): m/z 818.4 (M+1)⁺.

Following the steps described above and by substituting the bis-N-Boc protected pyrrolidine building block B-1C with the amide building block B-2A, the following analogs were prepared.

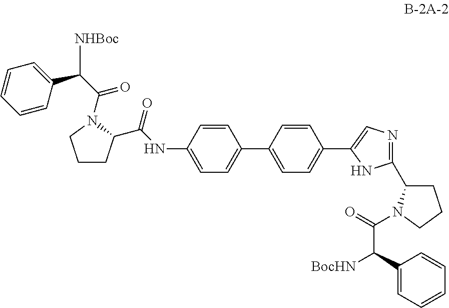

B-2A-2: ¹H NMR (500 MHz, d⁶-DMSO) δ 10.06 (m, 1H), 7.80 (d, J=8.0 Hz, 2H), 7.51-7.69 (m, 6H), 7.50 (m, 1H), 4.78-4.84 (m, 1H), 4.21-4.28 (m, 1H), 3.33-3.64 (m, 4H), 2.19 (m, 2H), 1.78-2.00 (m, 6H), 1.40 (s, 9H), 1.29 (s, 9H) ppm. LC-MS (ESI): m/z 602.3 (M+1)⁺.

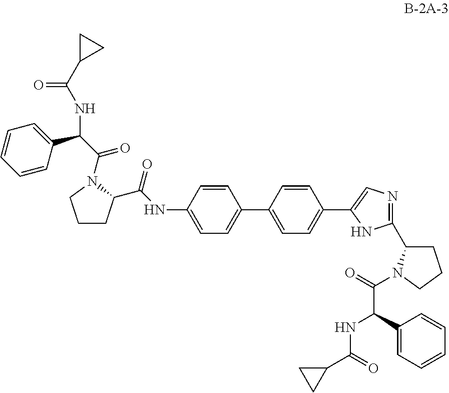

B-2A-3: ¹H NMR (500 MHz, CDCl₃) δ 9.05 (s, 1H), 7.73 (m, 4H), 7.50-7.73 (m, 4H), 7.48-7.49 (m, 4H), 7.38-7.43 (m, 6H), 6.82-6.88 (m, 2H), 5.60-5.68 (m, 2H), 5.37 (m, 1H), 4.78 (m, 1H), 3.92 (m, 1H), 3.84 (m, 1H), 3.28 (m, 2H), 2.82 (m, 1H), 2.45 (m, 1H), 2.05-2.15 (m, 4H), 1.85-1.95 (m, 4H), 1.45-1.46 (m, 2H), 0.95-0.98 (m, 4H), 0.82-0.85 (m, 2H), 0.74-0.78 (m, 4H) ppm.

Following the steps described above and by substituting the bis-N-Boc protected pyrrolidine building block B-1C with the amide building block B-3A, the following analogs were prepared.

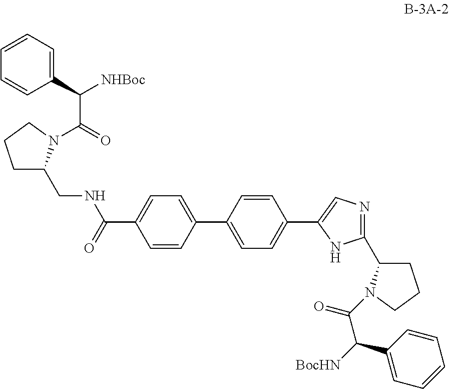

B-3A-2: LC-MS (ESI): m/z 882.5 (M+1)⁺.

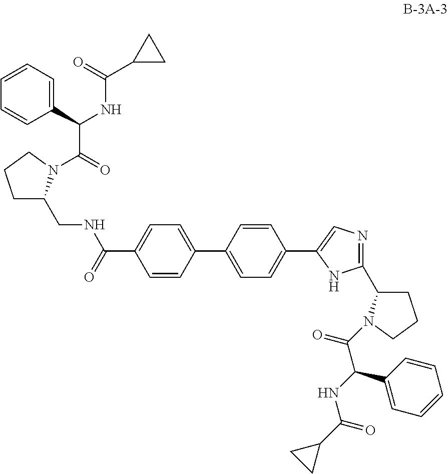

B-3A-3: LC-MS (ESI): m/z 818.3 (M+1)⁺.

Following the steps described above and by substituting the bis-N-Boc protected pyrrolidine building block B-1C with the benzylether building block B-4A, the following analogs were prepared.

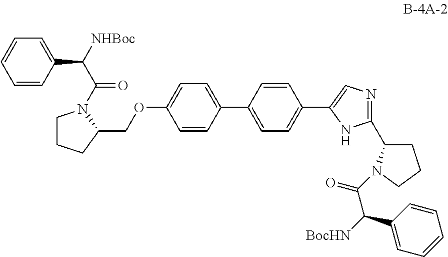

B-4A-2: ¹H NMR (500 MHz, d⁶-DMSO) δ 11.40 (m, 1H), 7.76-7.78)ₘ, 2H), 7.55-7.58 (m, 4H), 7.27-7.40 (m, 13H), 7.02 (m, 2H), 6.61 (m, 2H), 5.30-5.42 (m, 2H), 5.14 (m, 1H), 4.36 (m, 1H), 4.03-4.21 (m, 2H), 3.61-3.80 (m, 2H), 3.27 (m, 2H), 1.75-2.17 (m, 8H), 1.37 (m, 18H) ppm. LC-MS (ESI): m/z 855.5 (M+1)⁺.

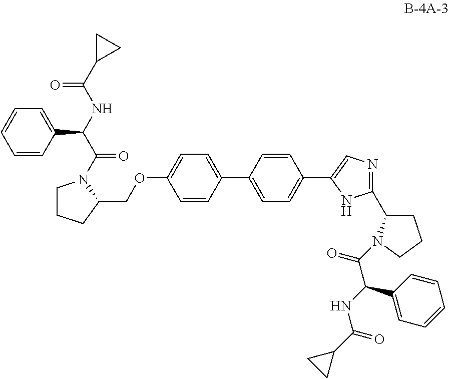

B-4A-3: ¹H NMR (500 MHz, CDCl₃) δ 10.64 and 10.40 (m, m, 1H), 7.77-7.81 and 7.65-7.69 (m, m, 2H), 7.34-7.60 (m, 14H), 7.16-7.19 (m, 1H), 7.04-7.07 (m, 3H), 6.83-6.84 and 6.70-6.71 (m, 1H), 5.73-5.75 (m, 1H), 5.64-5.66 and 5.58-5.60 (m, m, 1H), 5.31-5.37 (m, 1H), 4.46-4.50 (m, 1H), 4.31-4.33 (m, 1H), 4.04-4.08 (m, 1H), 3.77-3.85 (m, 1H), 3.61-3.55 (m, 1H), 3.24-3.26 (m, 1H), 3.07-3.09 (m, 1H), 2.91-2.95 (m, 1H), 2.80-2.82 (m, 1H), 2.00-2.15 (m, 6H), 1.87-1.93 (m, 2H), 1.72-1.78 (m, 1H), 1.38-1.48 (m, 2H), 0.86-1.03 (m, 4H), 0.68-0.83 (m, 4H) ppm. LC-MS (ESI): m/z 791.4 (M+1)⁺.

TABLE 1
Additional Analogs

| Com- | | Retention | |
|---|---|---|---|
| pound | | time | |
| # | Structure | (min, LC-MS) | (M + H)⁺ |
| B-1A-4 | 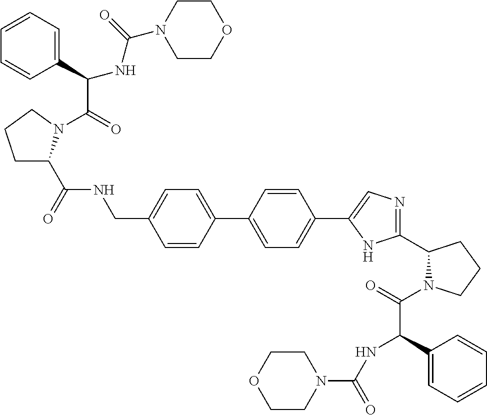 | 2.76 | 908 |
| B-1A-5 | 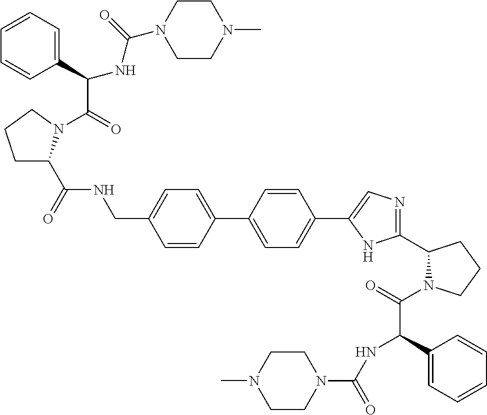 | 1.99 | 934 |
| B-1B-4 | 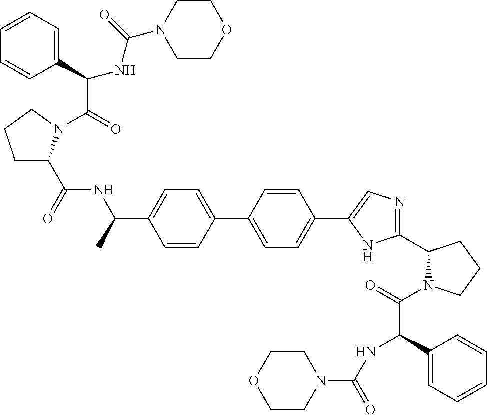 | 2.04 | 948 |
| B-1C-4 | 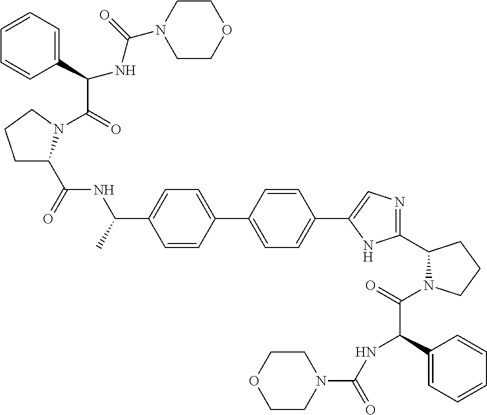 | 2.29 | 922 |
| B-1C-5 | 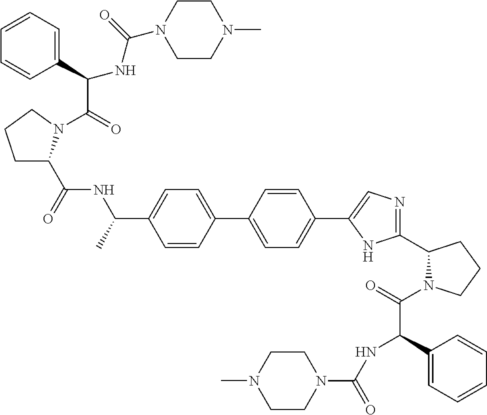 | 1.47 | 948 |
| B-2A-6 | 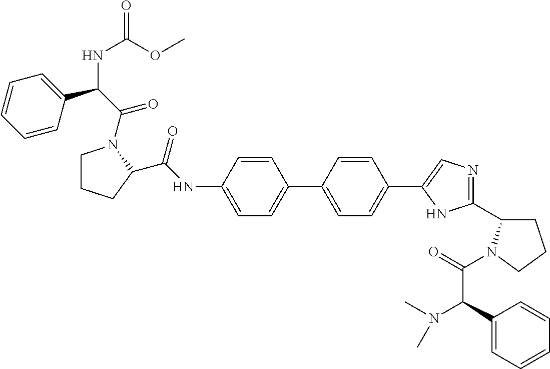 | 1.42 | 754 |
| B-2A-7 | 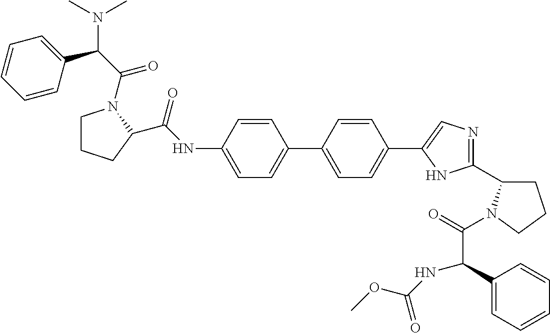 | 1.68 | 754 |
| B-2D-3 | 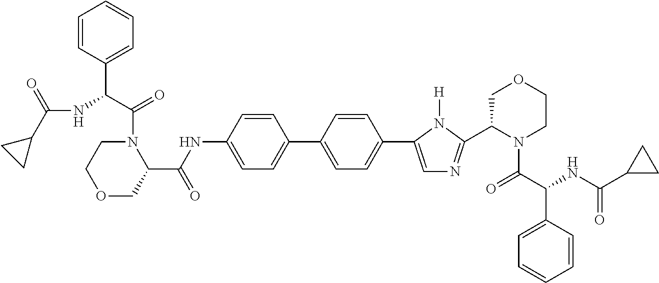 | | 836 |
| B-3A-4 | 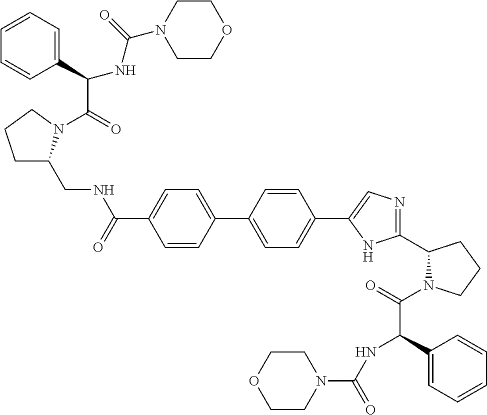 | 2.22 | 909 |
| B-3A-5 | 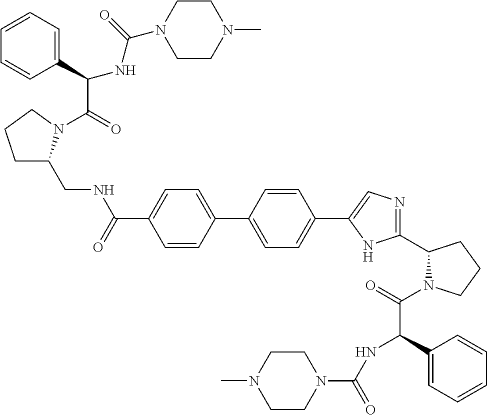 | 1.53 | 935 |
| B-4A-4 | 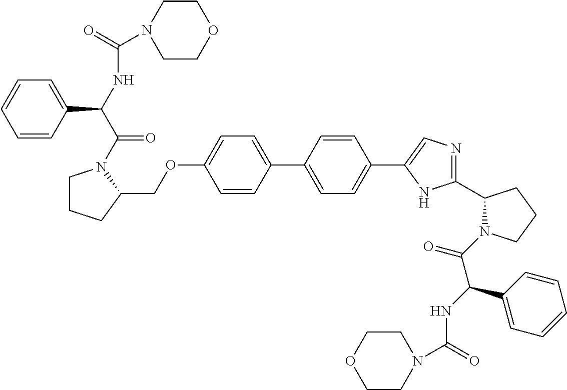 | | 881 |
| B-4A-5 | 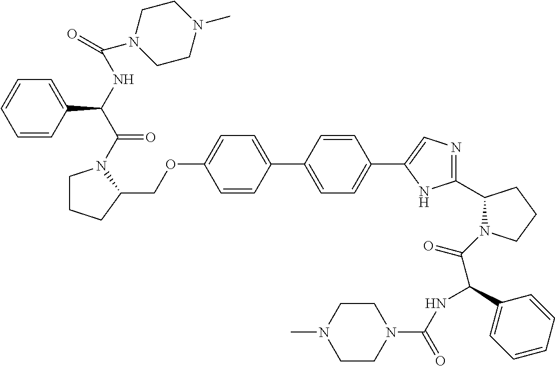 | 2.21 | 907 |

### Example 4

### Preparation Aryl Ether Analogs

Following the steps described above and by substituting the bis-N-Boc protected pyrrolidine building block B-1C with the amide building block B-5A, the following analogs were prepared.

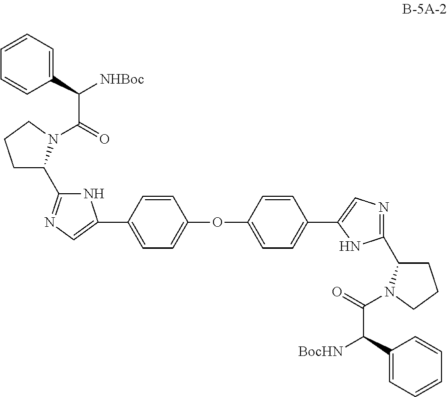

B-5A-2: ¹H NMR (500 MHz, CDCl₃) δ 7.64-7.66 (m, 4H), 7.36-7.45 (m, 10H), 7.14 (s, 2H), 7.01 (d, J=8.5 Hz, 4H), 5.75 (d, J=6.0 Hz, 2H), 5.36 (d, J=7.0 Hz, 2H), 5.31 (d, J=7.5 Hz, 2H), 3.78 (m, 2H), 3.22 (m, 2H), 2.85 (m, 2H), 2.08 (m, 2H), 2.02 (m, 2H), 1.90 (m, 2H), 1.42 (s, 18H) ppm. LC-MS (ESI): m/z 907.0 (M+1)⁺.

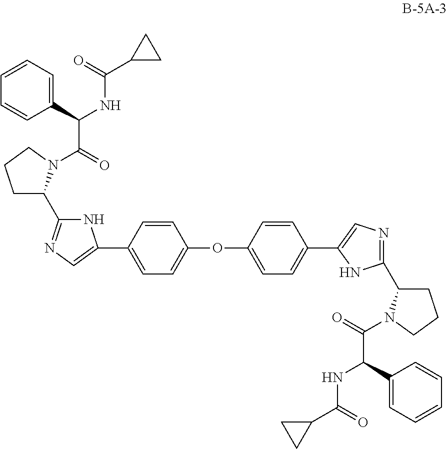

B-5A-3: ¹H NMR (500 MHz, CDCl₃) δ 7.65-7.67 (m, 4H), 7.38-7.47 (m, 10H), 7.16 (s, 2H), 7.01-7.03 (m, 6H), 5.59 (d, J=6.0 Hz, 2H), 5.32 (d, J=6.0 Hz, 2H), 3.84 (m, 2H), 3.26-3.27 (m, 2H), 2.73 (m, 2H), 2.07 (m, 4H), 1.90 (m, 2H), 1.45 (m, 2H), 0.89-0.94 (m, 6H), 0.73-0.74 (m, 4H) ppm. LC-MS (ESI): m/z 843.0 (M+1)⁺.

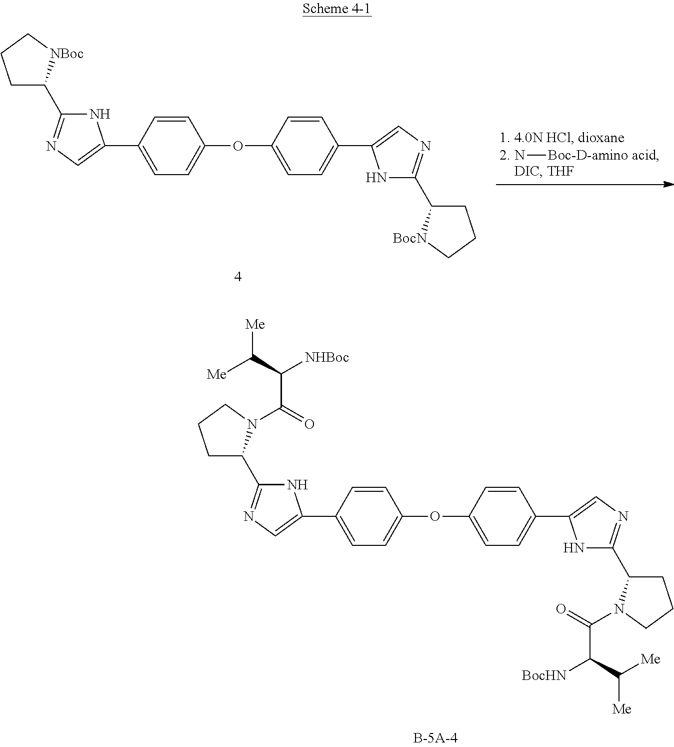

Step 1. Referring to Scheme 4-1, 15 mL 4.0 N HCl/dioxane was added dropwise to a stirred solution of 4 (1.5 g, 2.43 mmol) in 20 mL dioxane. The mixture was stirred at rt for 4 h, then concentrated to yield a yellowish solid (1.5 g), which was used directly for the next step.

Step 2. The obtained solid (500 mg, 0.81 mmol) was suspended in THF and 0.5 mL DIPEA was added slowly while stirring, followed by N-Boc-D-Valine (443 mg, 2.34 mmol). 15 min. later, N,N′-Diisopropylcarbodiimide was added dropwisely and the mixture was stirred at rt for 2 h. The solvent was evaporated and the residue was re-dissolved with EtOAc and filtered. The filtrate was concentrated to yield a residue which was purified by silica gel column chromatography (DCM/MeOH=100:1) to obtain B-5A-4 (300 mg, 47%): ¹H NMR (500 MHz, CDCl₃) δ 1.03 (d, 12H, J=6.5 Hz), 1.30-1.45 (m, 15H), 2.02-2.15 (m, 8H), 2.84 (m, 2H), 3.57-3.59 (m, 2H), 3.92-4.14 (m, 4H), 5.28-5.33 (m, 4H), 6.99 (d, 4H, J=8.0 Hz), 7.06 (s, 2H), 7.62 (brs, 4H) ppm; LCMS: Anal. Calcd. for C₄₆H₆₂N₈O₇ 838.47. Found 839.3 (M+H)¹; HPLC showed 100% purity. Retention time=16.85 min. 214 and 254 nm (UV detection wavelength).

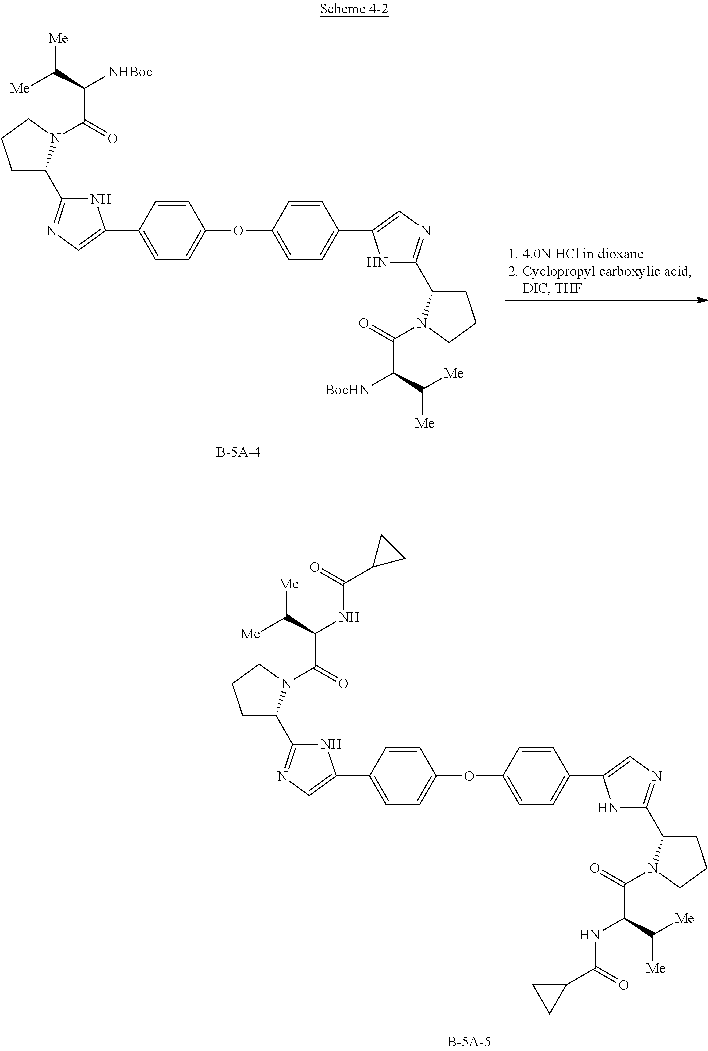

3 mL 4.0N HCl/dioxane was added dropwise to a stirred solution of B-5A-4 (150 mg, 0.18 mmol) in 5 mL dioxane, the mixture was stirred at rt for 4 h, then concentrated to yield a yellowish solid (132 mg), which was used directly for the next step. The solid (132 mg, 0.1788 mmol) was suspended in THF with stirring. DIPEA (00.1 mL) was added, followed by cyclopropanecarboxylic acid (67.6 mg, 0.54 mmol) and DIC. The mixture was stirred at rt for 2 h and concentrated. The mixture was re-dissolved in EtOAc, filtered and the filtrate was concentrated. The filtrate was purified by prep-HPLC to obtain target compd. B-5A-5 (30 mg, 22%). ¹H NMR (500 MHz, CDCl₃) δ 0.4-0.44 (m, 4H), 0.67-0.74 (m, 2H), 1.02-1.10 (m, 14H), 1.99-2.01 (m, 2H), 2.09-2.18 (m, 4H), 2.38-2.41 (m, 2H), 3.57-3.59 (m, 2H), 4.14-4.19 (m, 4H), 5.43 (d, 2H, J=7.5 Hz), 6.98-7.01 (m, 6H), 7.58-7.62 (m, 4H), 8.05 (brs, 1H) ppm; LCMS: Anal. Calcd. for C₄₄H₅₄N₈O₅ 774.4. Found 775.2 (M+H)¹; HPLC showed 100% purity. Retention time=14.81 min. 214 and 254 nm (UV detection wavelength).

Following the steps described above and by substituting the bis-N-Boc protected pyrrolidine building block B-1C with the amide building block B-6A, the following analogs were prepared.

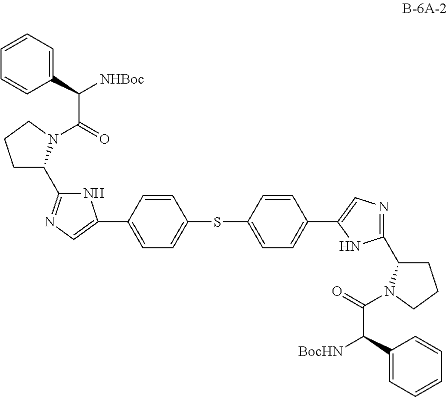

B-6A-2: LC-MS (ESI): m/z 924.4 (M+1)⁺.

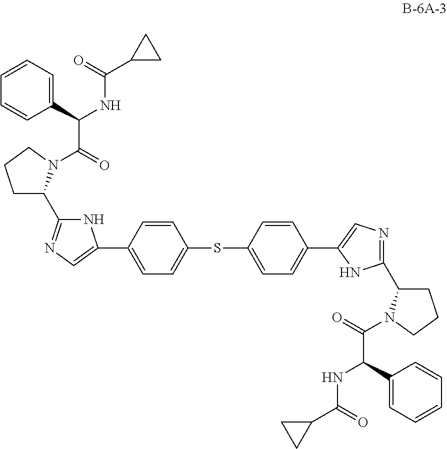

B-6A-3: LC-MS (ESI): m/z 859.4 (M+1)⁺.

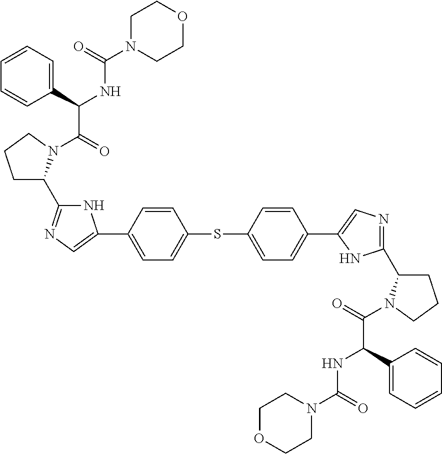

B-6A-4: LC-MS (ESI): m/z 949 (M+1)⁺.

The phenyl-benzimidazole containing core B-7A was prepared using similar procedures described for the synthesis of B-1A. The further derivatization of this core was achieved by following the steps described above and by substituting the bis-N-Boc protected pyrrolidine building block B-1C with the amide building block B-7A. The following analogs were prepared.

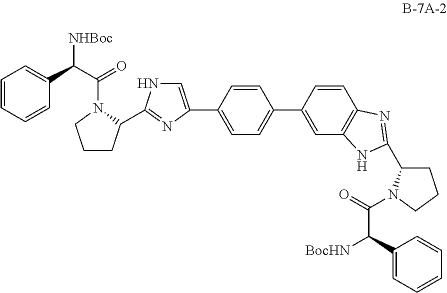

B-7A-2: ¹H NMR (500 MHz, CDCl₃) δ 11.02 and 11.50 (m, m, 2H), 7.71-7.76 (m, 2H), 7.63-7.64 (m, 2H), 7.36-7.56 (m, 11H), 7.25-7.27 (m, 2H), 5.33-5.78 (m, 6H), 3.68-3.85 (m, 2H), 3.21-3.31 (m, 2H), 3.05 and 2.92 (m, m, 2H), 1.97-2.21 (m, 6H), 1.44-1.49 (m, 18H) ppm. LC-MS (ESI): m/z 865.2 (M+1)⁺.

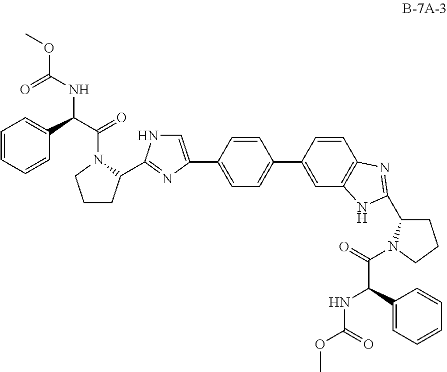

B-7A-3: (a pair of diastereomers) ¹H NMR (500 MHz, CDCl₃) δ 7.75 (m, 2H), 7.62-7.67 (m, 2H), 7.38-7.52 (m, 11H), 7.26-7.28 (m, 2H), 6.06-6.07 (m, 2H), 5.42-5.52 (m, 3H), 5.30-5.32 (m, 1H), 3.74-3.80 (m, 2H), 3.79 (s, 3H), 3.66 (s, 3H), 3.21-3.27 (m, 2H), 2.95 (m, 2H), 1.62-2.22 (m, 8H) ppm. LC-MS (ESI): m/z 781.0 (M+1)⁺.

Biological Activity

Biological activity of the compounds of the invention was determined using an HCV replicon assay. The HCV 1b_Huh-Luc/Neo-ET cell line persistently expressing a bicistronic genotype 1b replicon in Huh 7 cells was obtained from ReBLikon GMBH. This cell line was used to test compound inhibition using luciferase enzyme activity readout as a measurement of compound inhibition of replicon levels.

On Day 1 (the day after plating cells), each compound is added in triplicate to the cells. Plates are incubated for 72 h prior to determining luciferase levels. Enzyme activity was measured using a Bright-Glo Kit (cat. number E2620) manufactured by Promega Corporation. The following equation was used to generate a percent control value for each compound.

% Control=(Average Compound Value/Average Control)*100

The EC₅₀ value was determined using GraphPad Prism and the following equation:

Y=Bottom+(Top−Bottom)/(1+10̂((LogIC50−X)*HillSlope))

EC₅₀ values of compounds are determined several times in the replicon assay.

Example compounds of the disclosed invention are illustrated in Table 2. The table shows inhibitory activity of many of the example compounds with respect to HCV 1b. The biological activity is indicated as being *, **, *** or ****, which corresponds to EC₅₀ ranges of >1000 nM, 999 nM to 10 nM, 9.9 nM to 1 nM, or <1 nM respectively. The tables further provide mass spectrometry results for the synthesized example compounds.

Pharmaceutical Compositions

A tenth aspect of the invention provides a pharmaceutical composition comprising the compounds of the invention. In a first embodiment, the pharmaceutical composition further comprises one or more pharmaceutically acceptable excipients or vehicles, and optionally other therapeutic and/or prophylactic ingredients. Such excipients are known to those of skill in the art. The compounds of the present invention include, without limitation, basic compounds such as free bases. A thorough discussion of pharmaceutically acceptable excipients and salts is available in Remington's Pharmaceutical Sciences, 18th Edition (Easton, Pa.: Mack Publishing Company, 1990).

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers and optionally other therapeutic and/or prophylactic ingredients.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate and the like.

For oral administration, the composition will generally take the form of a tablet, capsule, a softgel capsule nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents and the like.

A eleventh aspect of the invention provides use of the compounds of the invention in the manufacture of a medicament.

In a first embodiment of the eleventh aspect the medicament is for the treatment of hepatitis C.

A twelfth aspect of the invention provides a method of treating hepatitis C comprising administering to a subject in need thereof, a therapeutically effective amount of a compound of the invention, optionally in a pharmaceutical composition. A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. The subject may be administered as many doses as is required to reduce and/or alleviate the signs, symptoms or causes of the disorder in question, or bring about any other desired alteration of a biological system. One of ordinary skill in the art of treating such diseases will be able, without undue experimentation and in reliance upon personal knowledge and the disclosure of this application, to ascertain a therapeutically effective amount of the compounds of this invention for a given disease.

Combination Therapy

The compounds of the present invention and their isomeric forms and pharmaceutically acceptable salts thereof are useful in treating and preventing HCV infection alone or when used in combination with other compounds targeting viral or cellular elements or functions involved in the HCV lifecycle. Classes of compounds useful in the invention may include, without limitation, all classes of HCV antivirals. For combination therapies, mechanistic classes of agents that may be useful when combined with the compounds of the present invention include, for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes and useful in the invention include, but are not limited to, macrocyclic, heterocyclic and linear HCV protease inhibitors such as telaprevir (VX-950), boceprevir (SCH-503034), narlaprevir (SCH-900518), ITMN-191 (R-7227), TMC-435350 (a.k.a. TMC-435), MK-7009, BI-201335, BI-2061 (ciluprevir), BMS-650032, ACH-1625, ACH-1095 (HCV NS4A protease co-factor inhibitor), VX-500, VX-813, PHX-1766, PHX2054, IDX-136, IDX-316, ABT-450 EP-013420 (and congeners) and VBY-376; the Nucleosidic HCV polymerase (replicase) inhibitors useful in the invention include, but are not limited to, R7128, PSI-7851, IDX-184, IDX-102, R1479, UNX-08189, PSI-6130, PSI-938 and PSI-879 and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nuclosidic HCV polymerase (replicase) inhibitors useful in the invention, include, but are not limited to, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, NS5A inhibitors of the present invention may be used in combination with cyclophyllin and immunophyllin antagonists (eg, without limitation, DEBIO compounds, NM-811 as well as cyclosporine and its derivatives), kinase inhibitors, inhibitors of heat shock proteins (e.g., HSP90 and HSP70), other immunomodulatory agents that may include, without limitation, interferons (-alpha, -beta, -omega, -gamma, -lambda or synthetic) such as Intron A™, Roferon-A™, Canferon-A300™, Advaferon™, Infergen™, Humoferon™, Sumiferon MP™, Alfaferone™, IFN-β™, Feron™ and the like; polyethylene glycol derivatized (pegylated) interferon compounds, such as PEG interferon-α-2a (Pegasys™), PEG interferon-α-2b (PEGIntron™), pegylated IFN-α-con1 and the like; long acting formulations and derivatizations of interferon compounds such as the albumin-fused interferon, Albuferon™, Locteron™ and the like; interferons with various types of controlled delivery systems (e.g. ITCA-638, omega-interferon delivered by the DUROS™ subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effective amount of a TNF-α antagonist. Exemplary, non-limiting TNF-α antagonists that are suitable for use in such combination therapies include ENBREL™, REMICADE™ and HUMIRA™.

In addition, NS5A inhibitors of the present invention may be used in combination with antiprotozoans and other antivirals thought to be effective in the treatment of HCV infection, such as, without limitation, the prodrug nitazoxanide. Nitazoxanide can be used as an agent in combination the compounds disclosed in this invention as well as in combination with other agents useful in treating HCV infection such as peginterferon alfa-2a and ribavarin (see, for example, Rossignol, J F and Keeffe, E B, Future Microbiol. 3:539-545, 2008).

NS5A inhibitors of the present invention may also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. No. 5,807,876; U.S. Pat. No. 6,498,178; U.S. Pat. No. 6,344,465; U.S. Pat. No. 6,054,472; WO97/40028; WO98/40381; WO00/56331, WO 02/04425; WO 03/007945; WO 03/010141; WO 03/000254; WO 01/32153; WO 00/06529; WO 00/18231; WO 00/10573; WO 00/13708; WO 01/85172; WO 03/037893; WO 03/037894; WO 03/037895; WO 02/100851; WO 02/100846; EP 1256628; WO 99/01582; WO 00/09543; WO02/18369; WO98/17679, WO00/056331; WO 98/22496; WO 99/07734; WO 05/073216, WO 05/073195 and WO 08/021,927.

Additionally, combinations of, for example, ribavirin and interferon, may be administered as multiple combination therapy with at least one of the compounds of the present invention. The present invention is not limited to the aforementioned classes or compounds and contemplates known and new compounds and combinations of biologically active agents (see, Strader, D. B., Wright, T., Thomas, D. L. and Seeff, L. B., AASLD Practice Guidelines. 1-22, 2009 and Manns, M. P., Foster, G. R., Rockstroh, J. K., Zeuzem, S., Zoulim, F. and Houghton, M., Nature Reviews Drug Discovery. 6:991-1000, 2007, Pawlotsky, J-M., Chevaliez, S, and McHutchinson, J. G., Gastroenterology. 132:179-1998, 2007, Lindenbach, B. D. and Rice, C. M., Nature 436:933-938, 2005, Klebl, B. M., Kurtenbach, A., Salassidis, K., Daub, H. and Herget, T., Antiviral Chemistry & Chemotherapy. 16:69-90, 2005, Beaulieu, P. L., Current Opinion in Investigational Drugs. 8:614-634, 2007, Kim, S-J., Kim, J-H., Kim, Y-G., Lim, H-S, and Oh, W-J., The Journal of Biological Chemistry. 48:50031-50041, 2004. Okamoto, T., Nishimura, Y., Ichimura, T., Suzuki, K., Miyamura, T., Suzuki, T., Moriishi, K. and Matsuura, Y., The EMBO Journal. 1-11, 2006, Soriano, V., Peters, M. G. and Zeuzem, S. Clinical Infectious Diseases. 48:313-320, 2009, Huang, Z., Murray, M. G. and Secrist, J. A., Antiviral Research. 71:351-362, 2006 and Neyts, J., Antiviral Research. 71:363-371, 2006, each of which is incorporated by reference in their entirety herein). It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent (for example, where each treatment comprises a different compound of the invention or where one treatment comprises a compound of the invention and the other comprises one or more biologically active agents) or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

TABLE 2

| Compound | | Inhibition of HCV | MS |
|---|---|---|---|
| ID | Structure | genotype 1b | (M + H)⁺ |
| 1 | 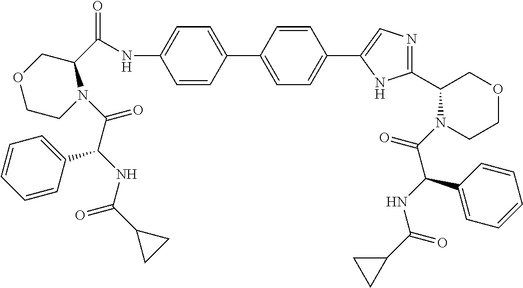 | **** | 836.4 |
| 2 | 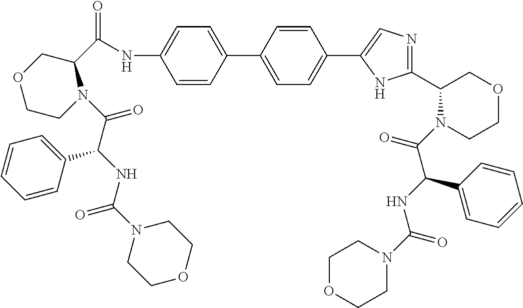 | *** | 926.4 |
| 3 | 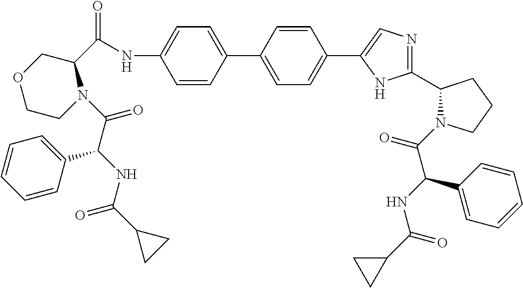 | **** | 820.4 |
| 4 | 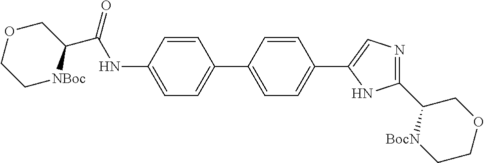 | * | 634.3 |
| 5 | 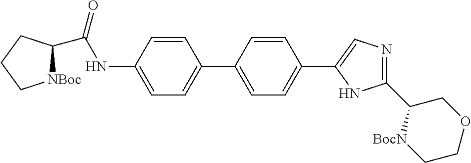 | * | 618.3 |
| 6 | 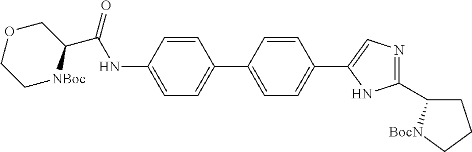 | ** | 618.3 |
| 7 | 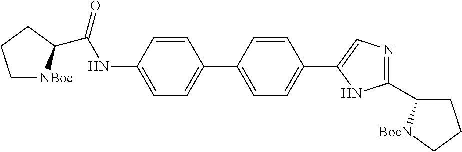 | * | 603.3 |
| 8 | 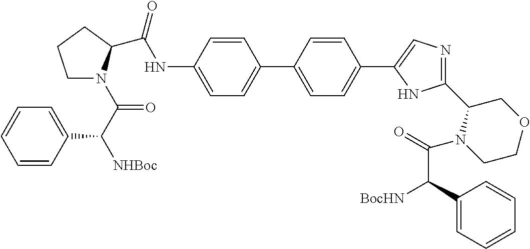 | **** | 884.4 |
| 9 | 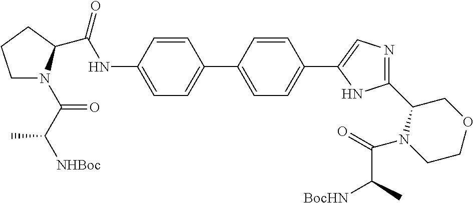 | ** | 760.4 |
| 10 | 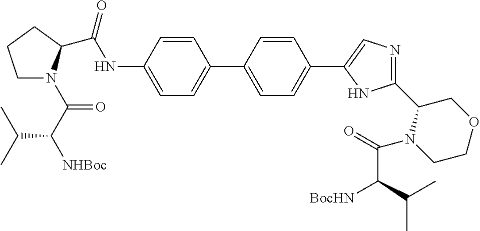 | ** | 816.5 |
| 11 | 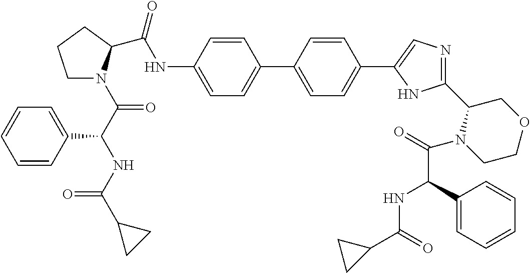 | **** | 820.4 |
| 12 | 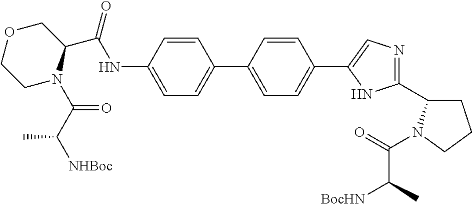 | ** | 760.4 |
| 13 | 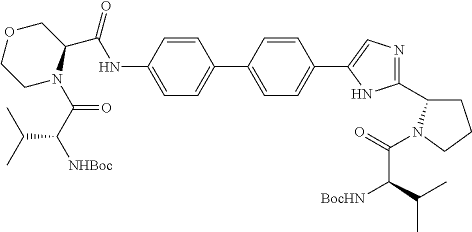 | ** | 816.5 |
| 14 | 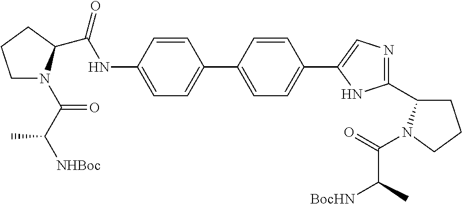 | *** | 744.4 |
| 15 | 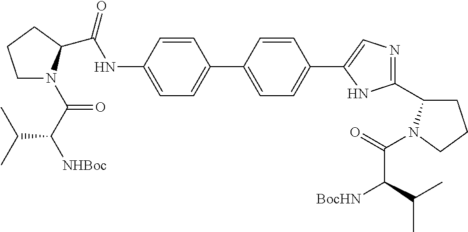 | *** | 800.5 |
| 16 | 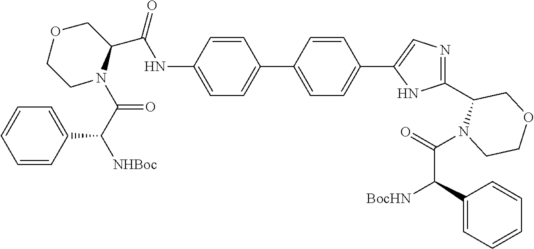 | **** | 900.4 |
| 17 | 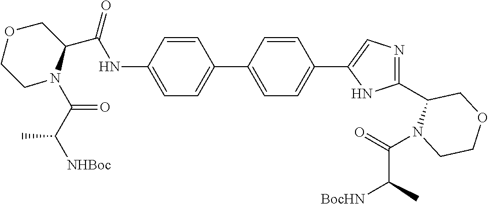 | ** | 776.4 |
| 18 | 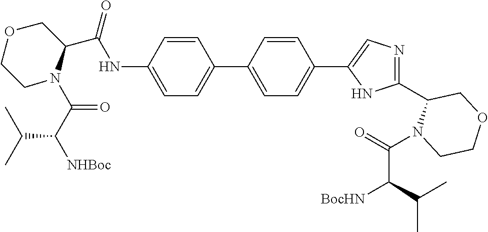 | ** | 832.5 |
| 19 | 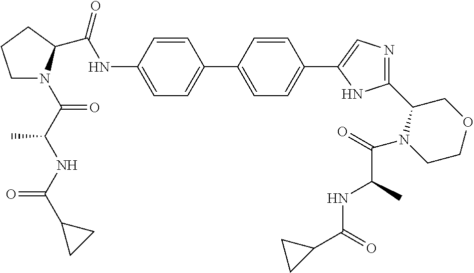 | *** | 696.3 |
| 20 | 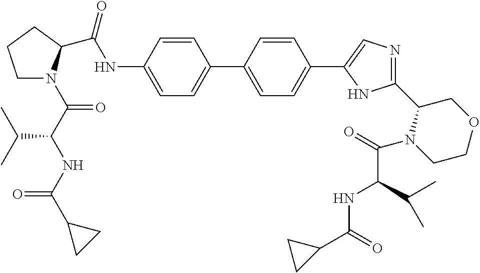 | *** | 752.4 |
| 21 | 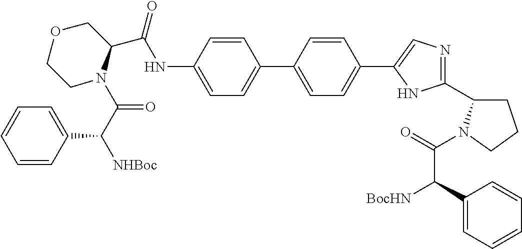 | **** | 884.4 |
| 22 | 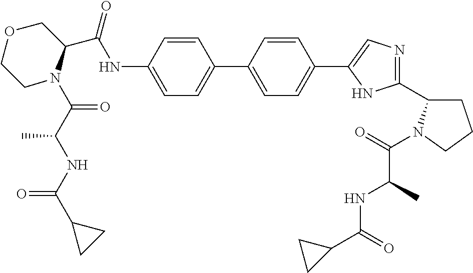 | *** | 696.4 |
| 23 | 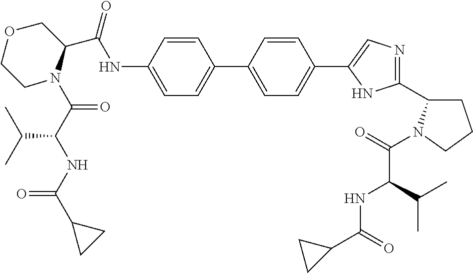 | *** | 752.4 |
| 24 | 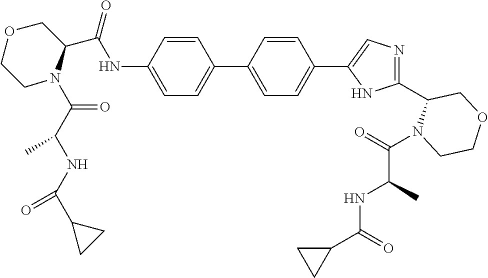 | ** | 712.3 |
| 25 | 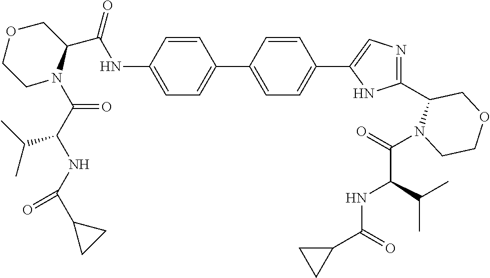 | *** | 768.4 |
| 26 | 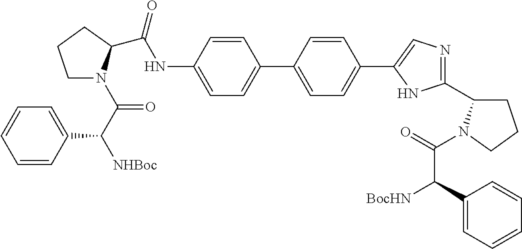 | **** | 868.4 |
| 27 | 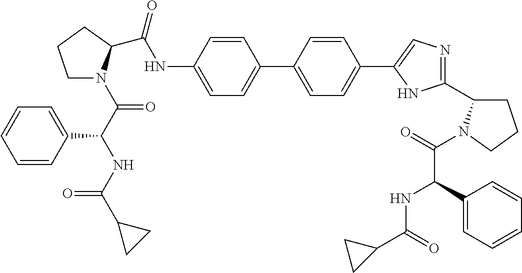 | **** | 804.4 |
| 28 | 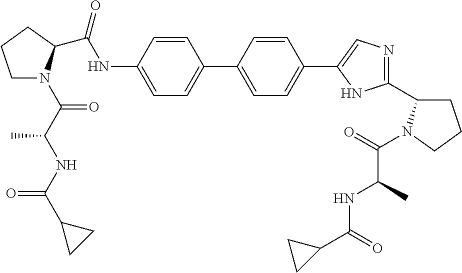 | *** | 680.3 |
| 29 | 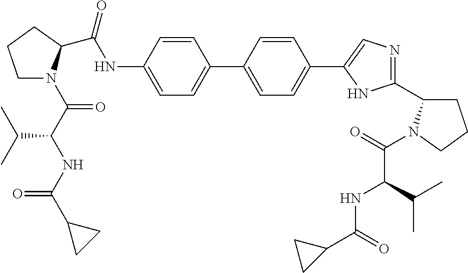 | *** | 736.4 |
| 30 | 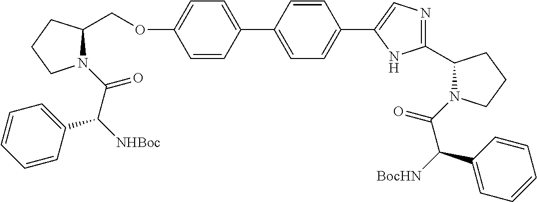 | **** | 855.4 |
| 31 | 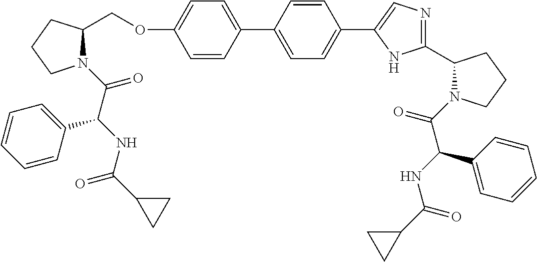 | **** | 791.4 |
| 32 | 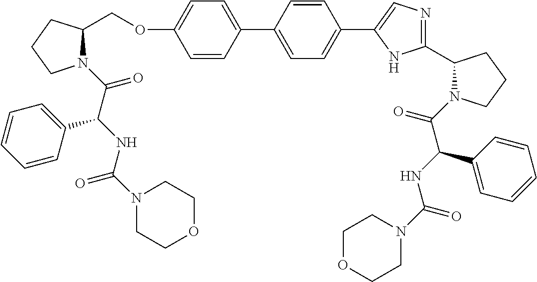 | **** | 881.4 |
| 33 | 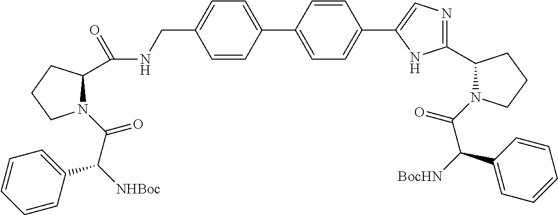 | **** | 882.4 |
| 34 | 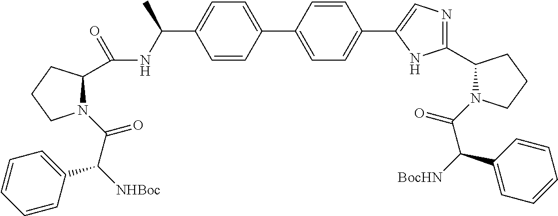 | **** | 896.5 |
| 35 | 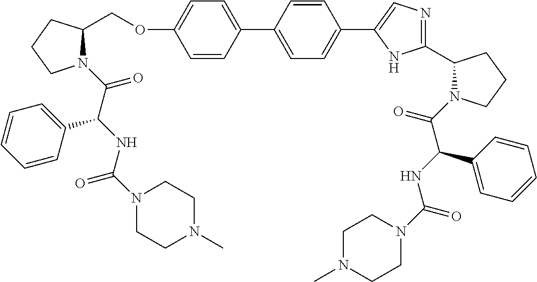 | *** | 907.5 |
| 36 | 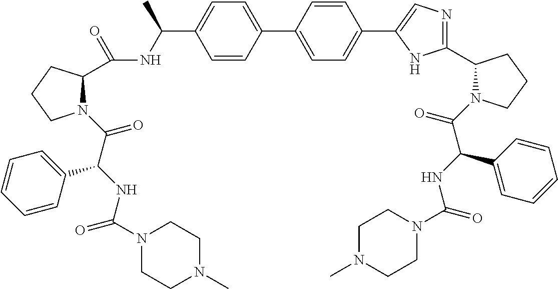 | *** | 948.5 |
| 37 | 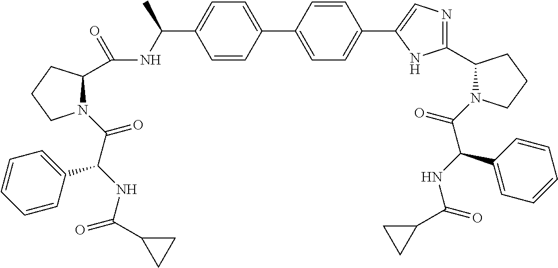 | **** | 832.4 |
| 38 | 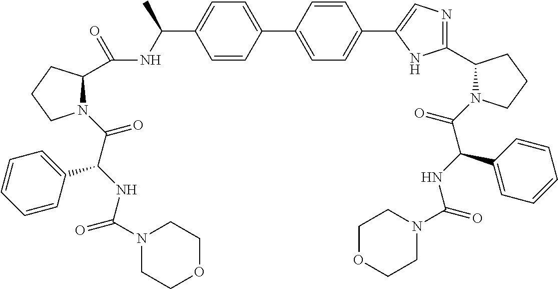 | *** | 922.5 |
| 39 | 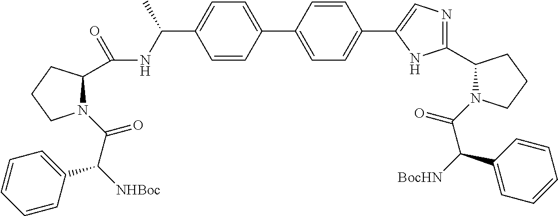 | **** | 896.5 |
| 40 | 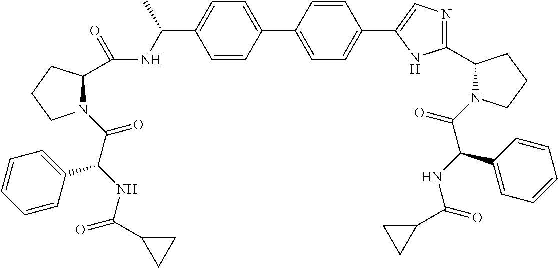 | **** | 832.4 |
| 41 | 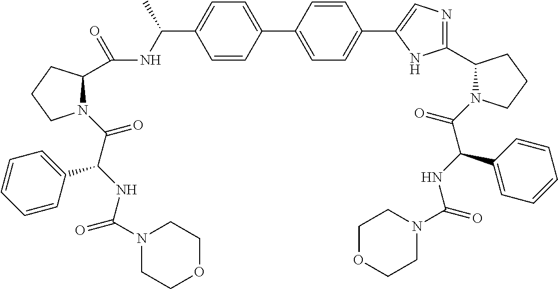 | *** | 922.5 |
| 42 | 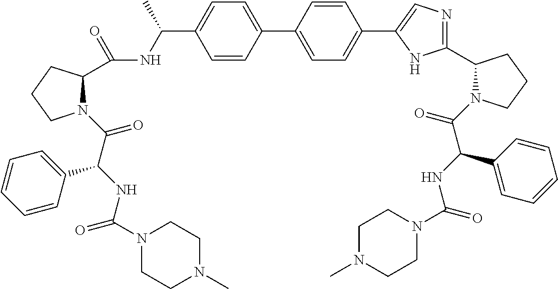 | *** | 948.5 |
| 43 | 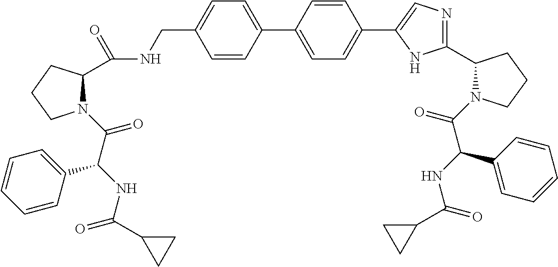 | **** | 818.4 |
| 44 | 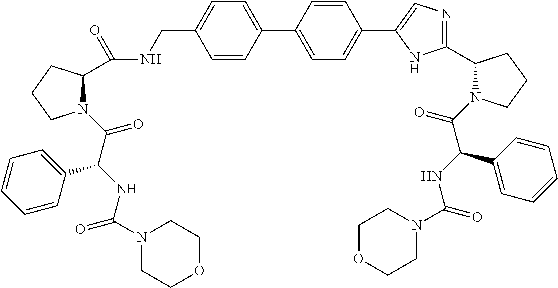 | *** | 908.4 |
| 45 | 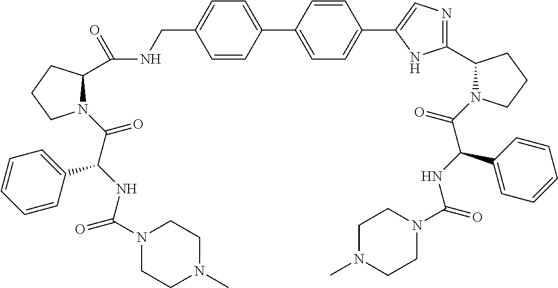 | *** | 934.5 |
| 46 | 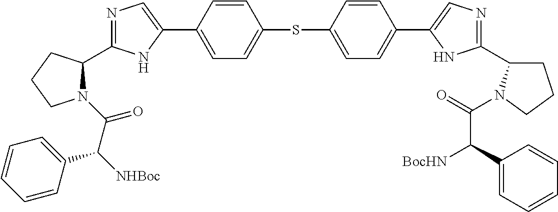 | **** | 923.4 |
| 47 | 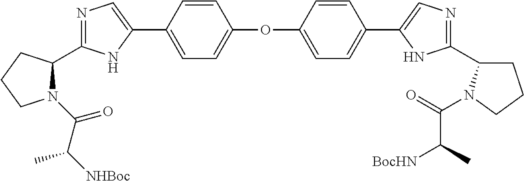 | *** | 783.4 |
| 48 | 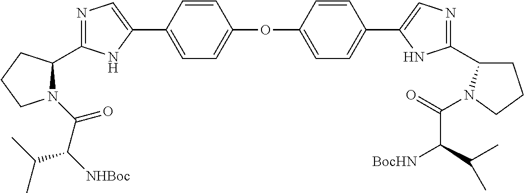 | *** | 839.5 |
| 49 | 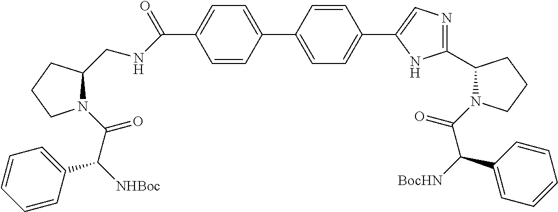 | **** | 882.4 |
| 50 | 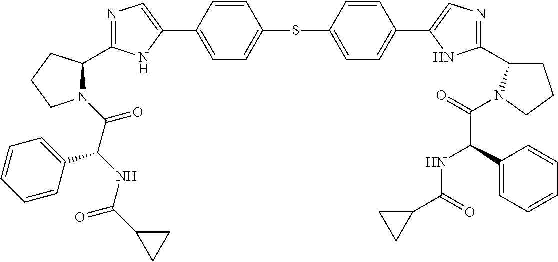 | **** | 859.4 |
| 51 | 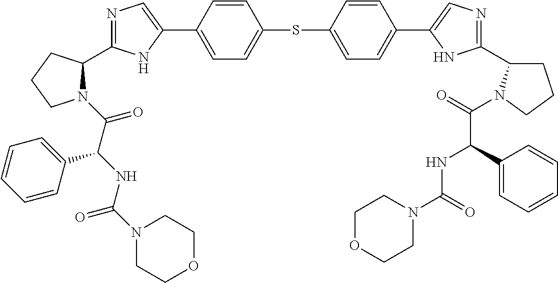 | **** | 949.4 |
| 52 | 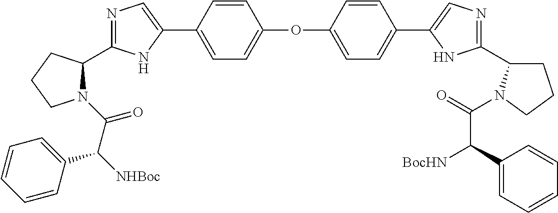 | **** | 907.4 |
| 53 | 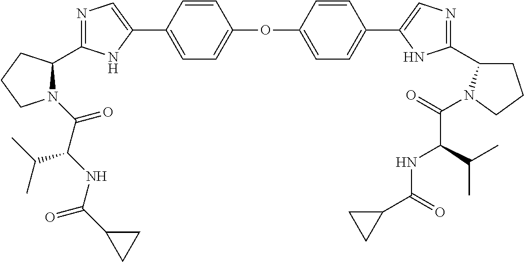 | *** | 775.4 |
| 54 | 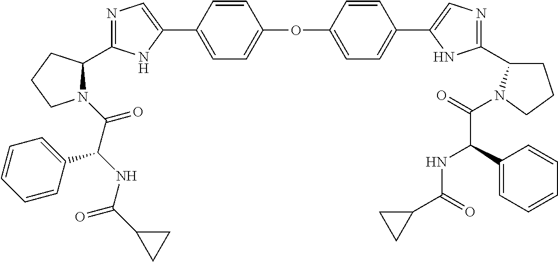 | **** | 843.4 |
| 55 | 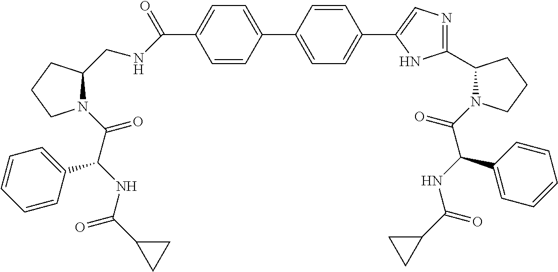 | **** | 818.4 |
| 56 | 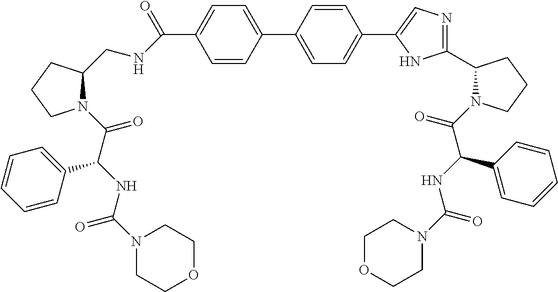 | **** | 908.4 |
| 57 | 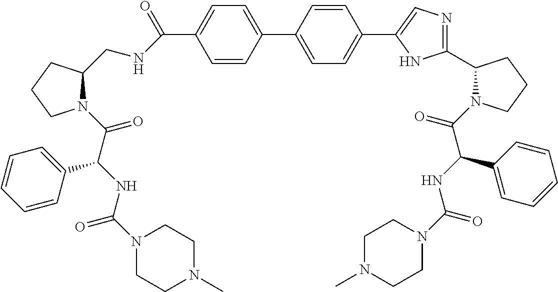 | *** | 934.5 |
| 58 | 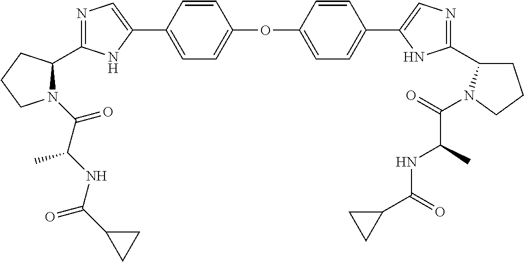 | ** | 719.4 |
| 59 | 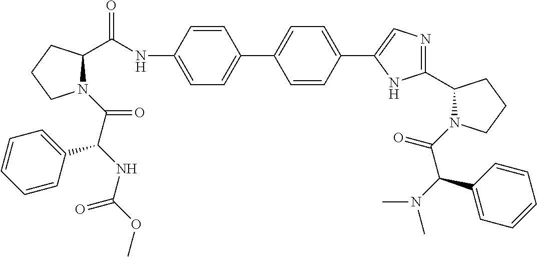 | **** | 754.4 |
| 60 | 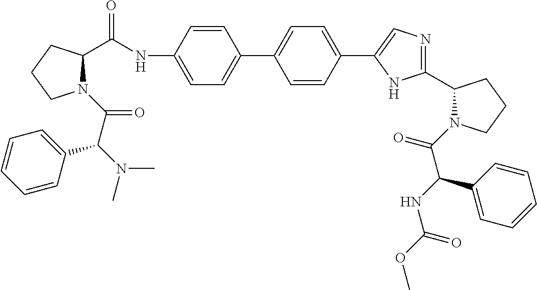 | **** | 754.4 |
| 61 | 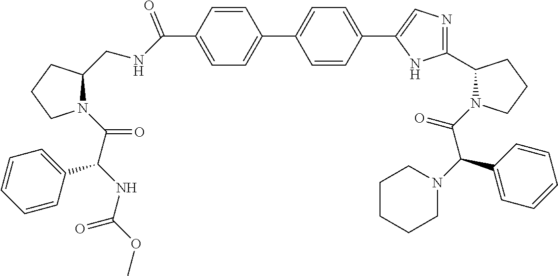 | **** | 808.4 |
| 62 | 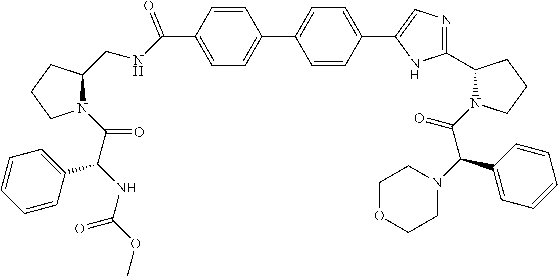 | **** | 810.4 |
| 63 | 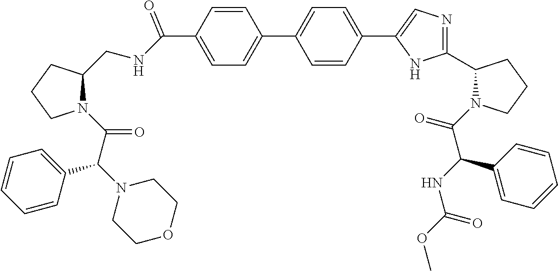 | **** | 810.4 |
| 64 | 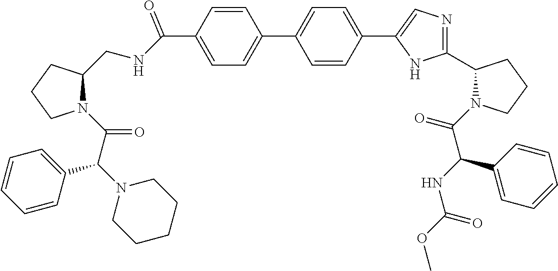 | **** | 808.4 |
| 65 | 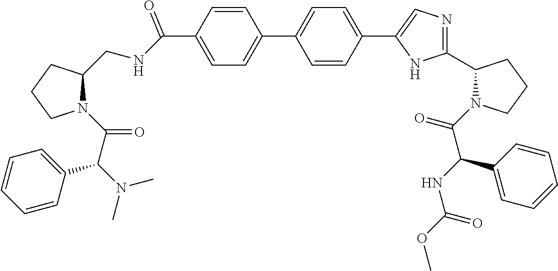 | **** | 768.4 |
| 66 | 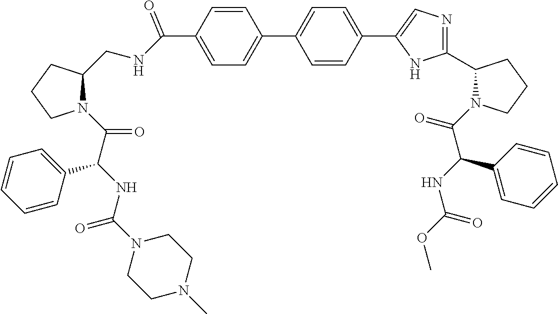 | **** | 866.4 |
| 67 | 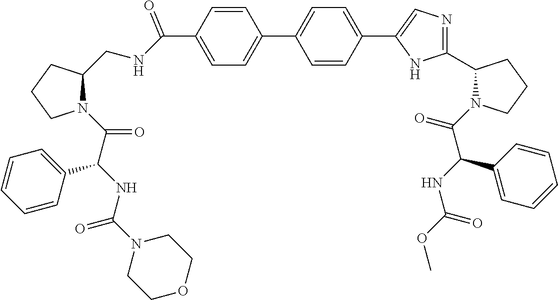 | **** | 853.4 |
| 68 | 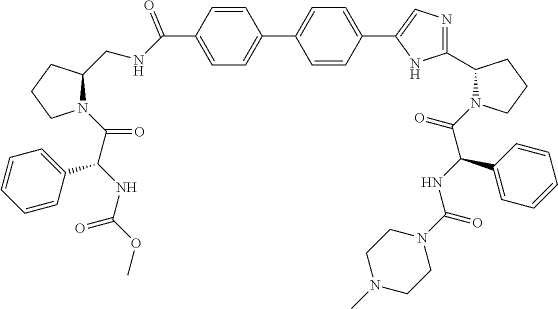 | **** | 866.4 |
| 69 | 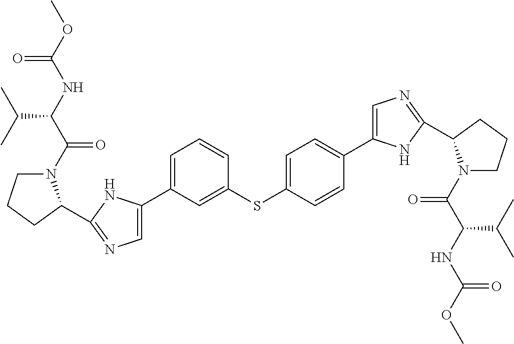 | **** | 771.4 |
| 70 | 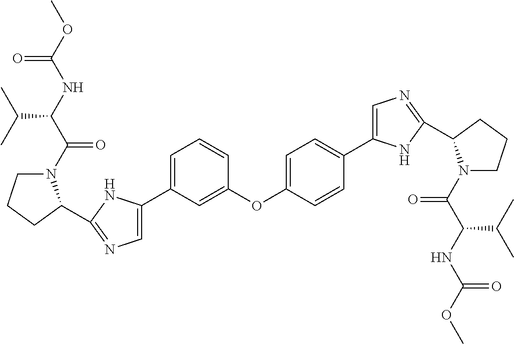 | **** | 755.4 |
| 71 | 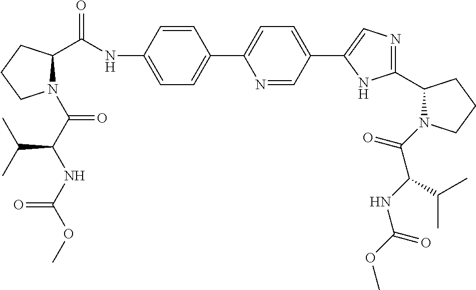 | *** | 717.4 |
| 72 | 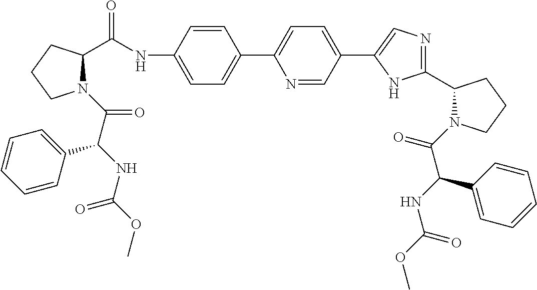 | **** | 785.3 |
| 73 | 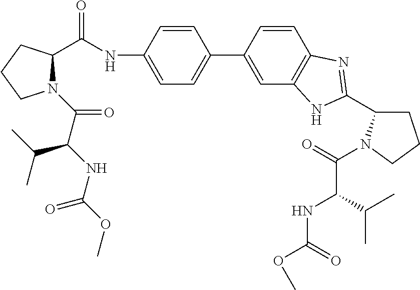 | *** | 690.4 |
| 74 | 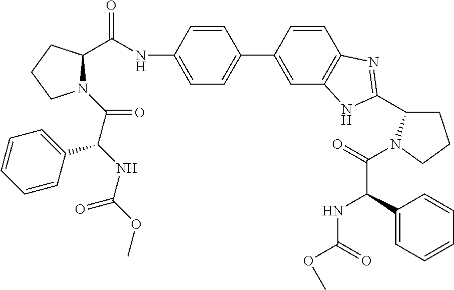 | **** | 758.3 |
| 75 | 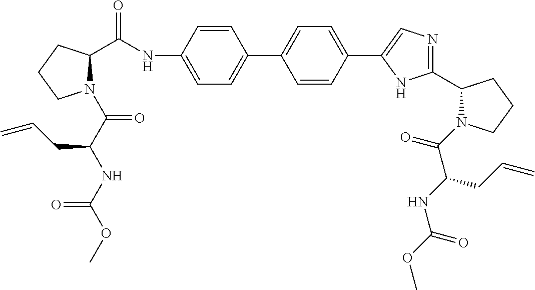 | *** | 712.3 |
| 76 | 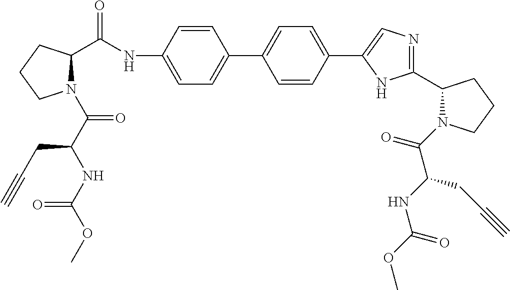 | *** | 708.3 |
| 77 | 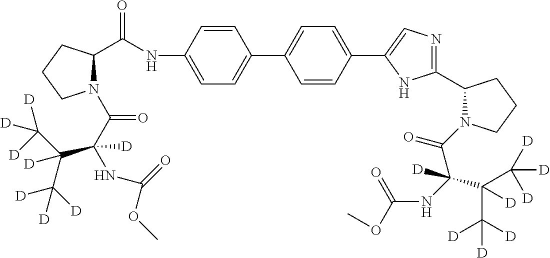 | **** | 732.5 |
| 78 | 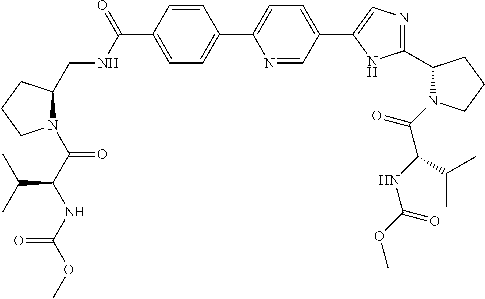 | **** | 731.4 |
| 79 | 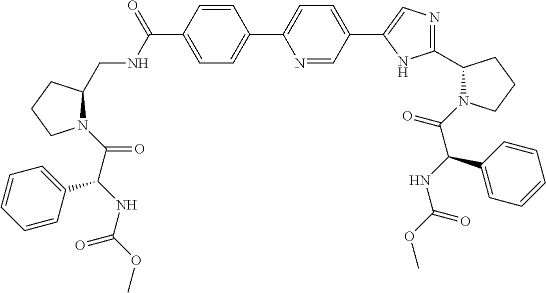 | **** | 799.3 |
| 80 | 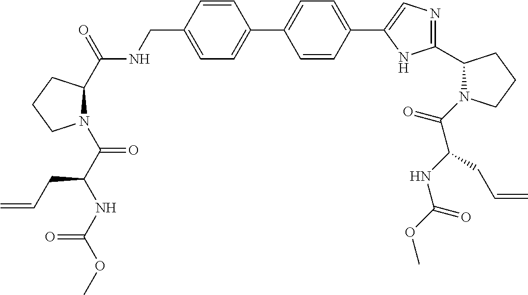 | *** | 726.4 |
| 81 | 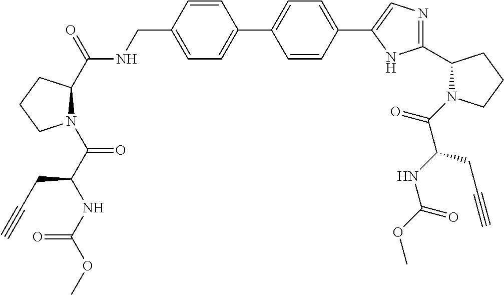 | ** | 722.3 |
| 82 | 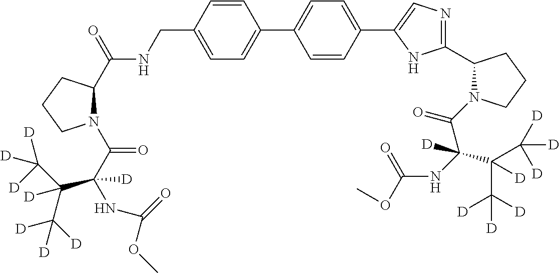 | *** | 746.5 |
| 83 | 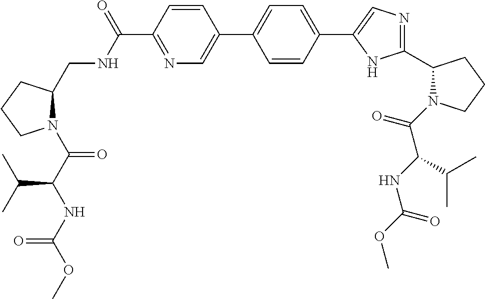 | *** | 731.4 |
| 84 | 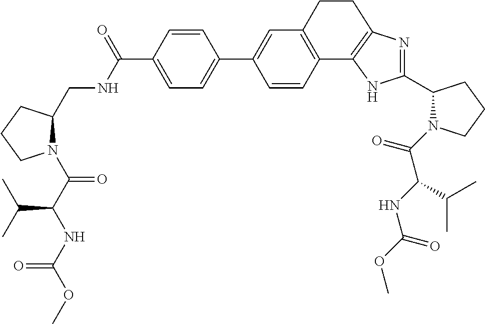 | **** | 756.4 |
| 85 | 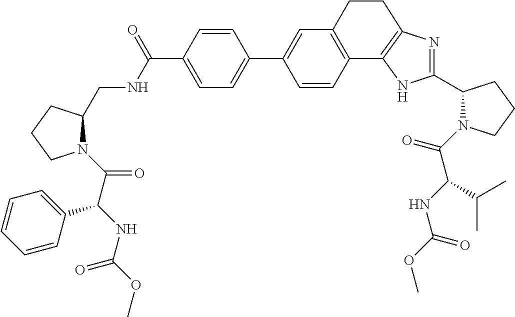 | **** | 790.4 |
| 86 | 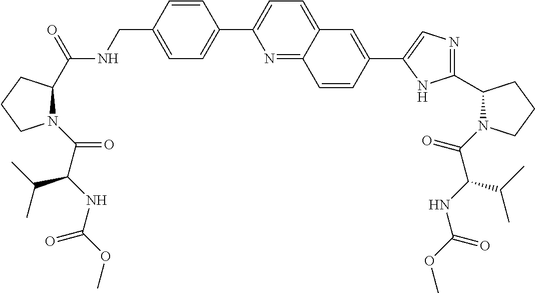 | *** | 781.4 |
| 87 | 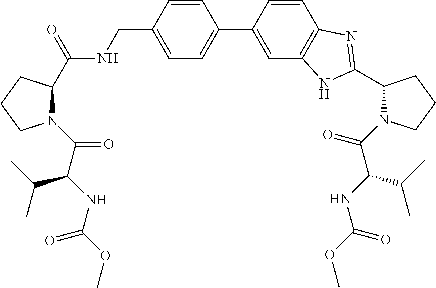 | *** | 704.4 |
| 88 | 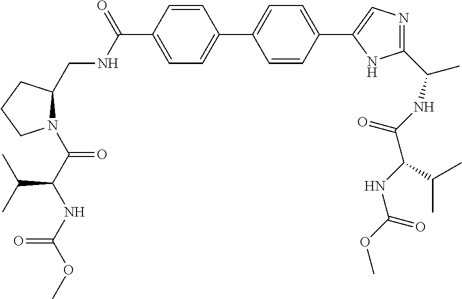 | **** | 704.4 |
| 89 | 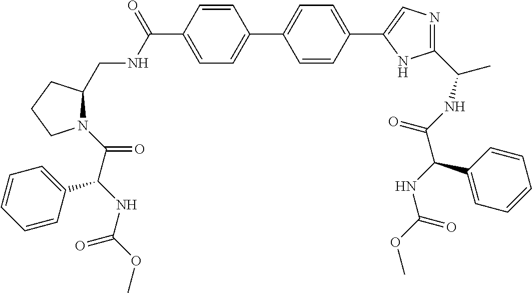 | **** | 772.3 |
| 90 | 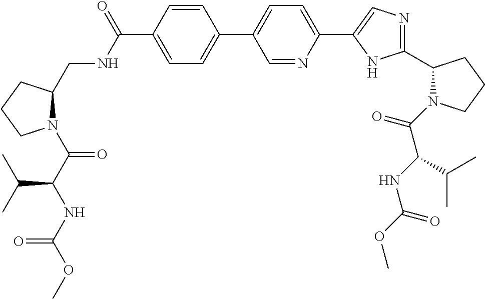 | *** | 731.4 |
| 91 | 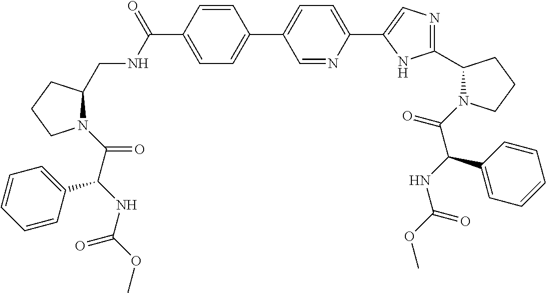 | **** | 799.3 |
| 92 | 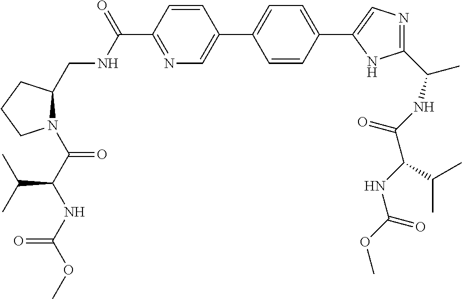 | *** | 705.4 |
| 93 | 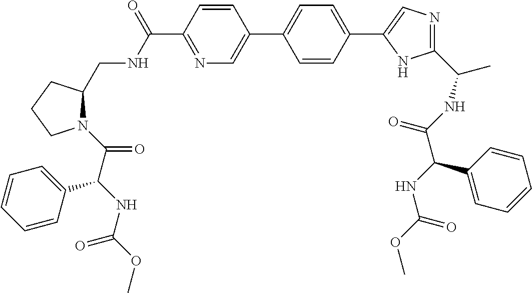 | **** | 773.3 |
| 94 | 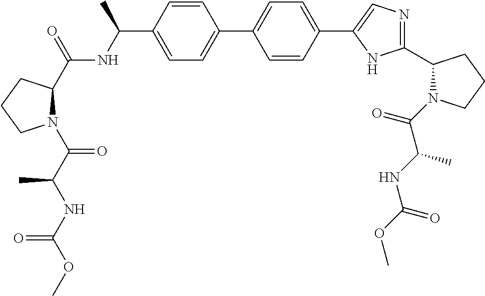 | *** | 688.3 |
| 95 | 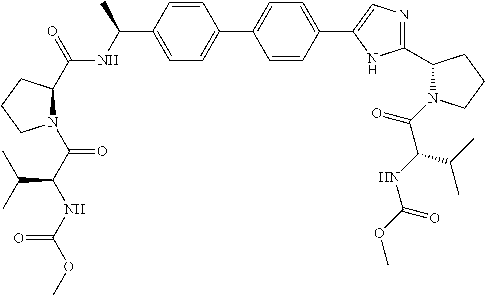 | **** | 744.4 |
| 96 | 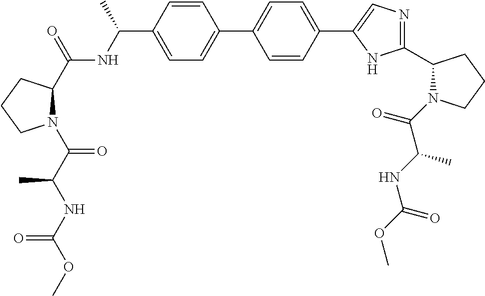 | *** | 688.3 |
| 97 | 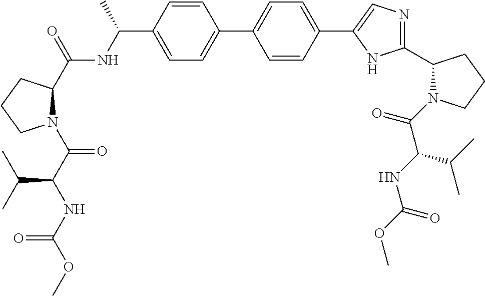 | *** | 744.4 |
| 98 | 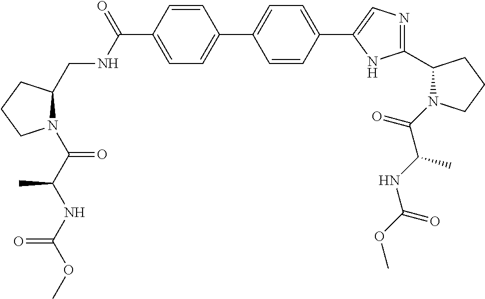 | *** | 674.3 |
| 99 | 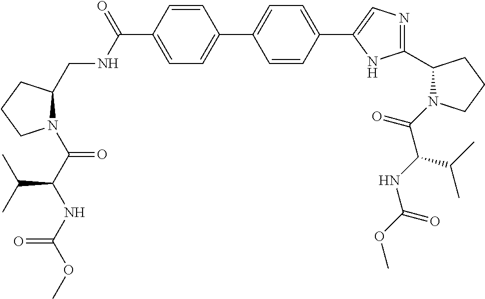 | **** | 730.4 |
| 100 | 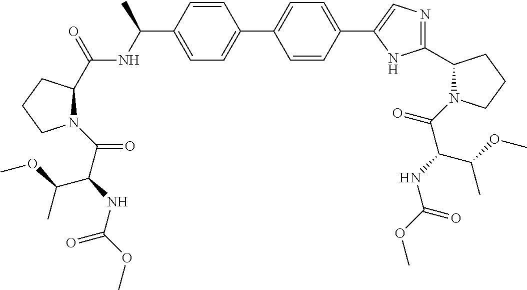 | **** | 776.4 |
| 101 | 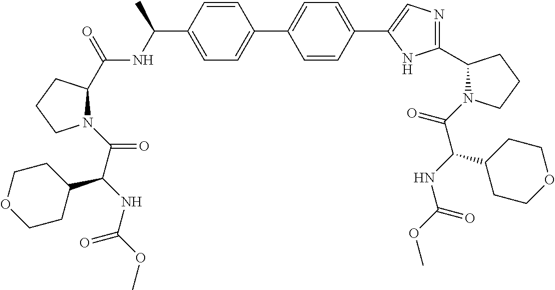 | *** | 828.4 |
| 102 | 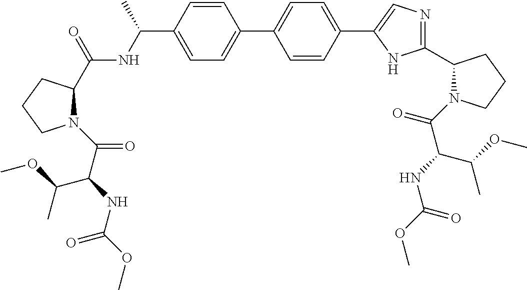 | ** | 776.4 |
| 103 | 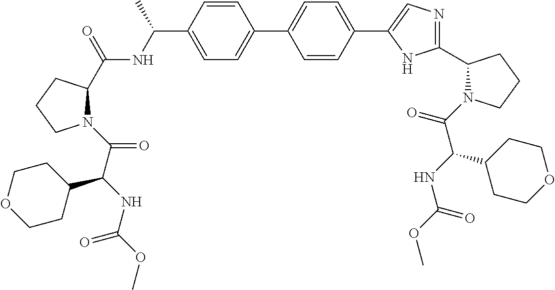 | ** | 828.4 |
| 104 | 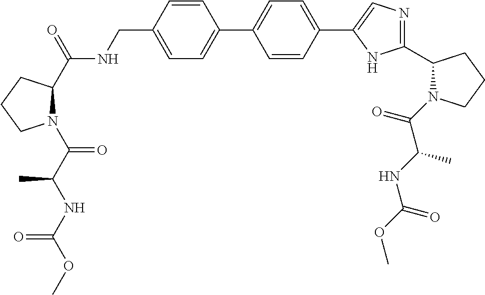 | ** | 674.3 |
| 105 | 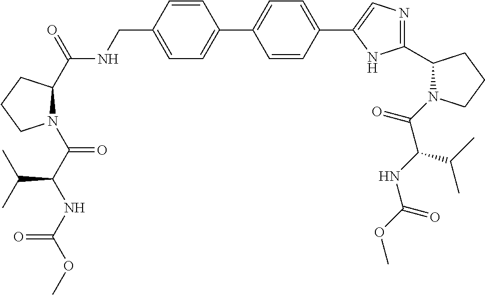 | ** | 730.4 |
| 106 | 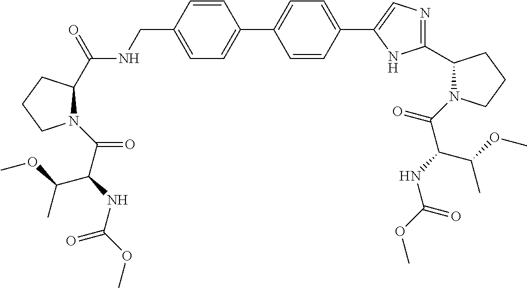 | ** | 762.4 |
| 107 | 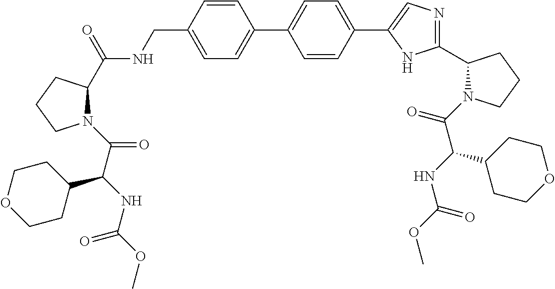 | ** | 814.4 |
| 108 | 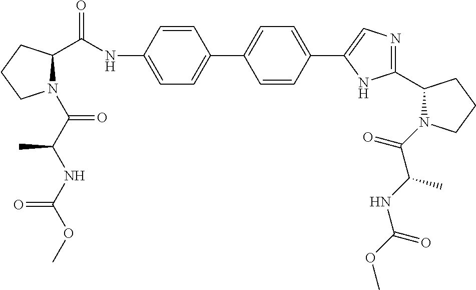 | ** | 660.3 |
| 109 | 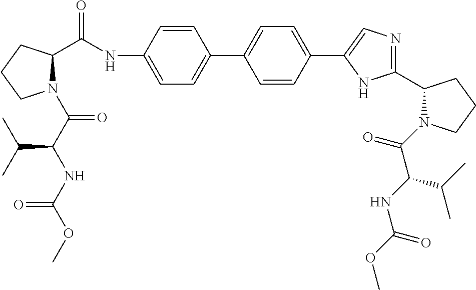 | ** | 716.4 |
| 110 | 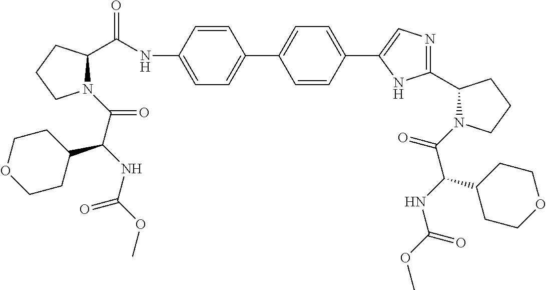 | *** | 800.4 |
| 111 | 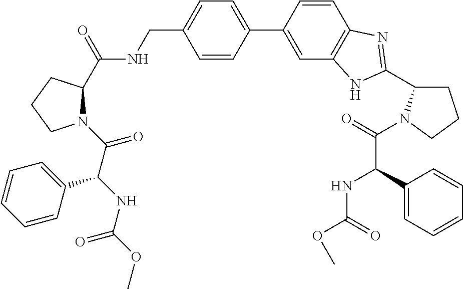 | **** | 772.3 |
| 112 | 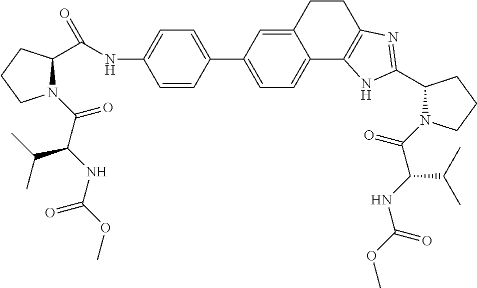 | *** | 742.4 |
| 113 | 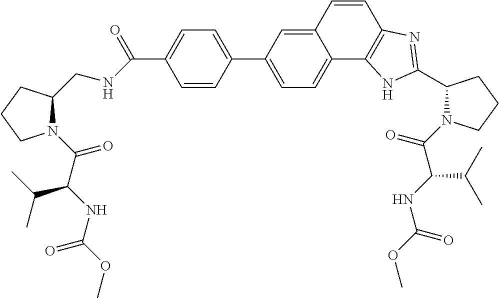 | **** | 754.4 |
| 114 | 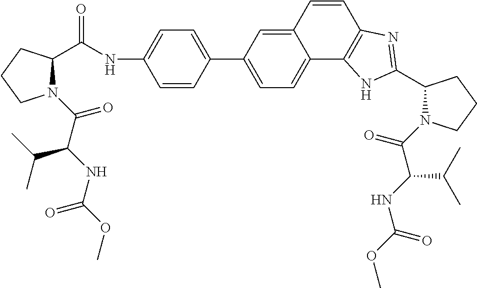 | **** | 740.4 |
| 115 | 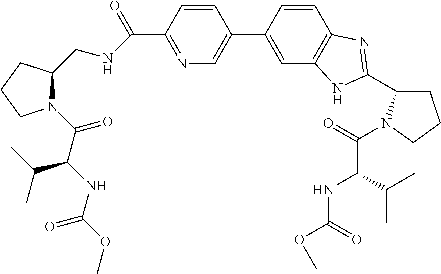 | ** | 705.4 |
| 116 | 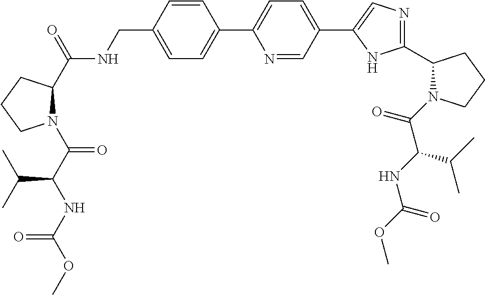 | ** | 731.4 |
| 117 | 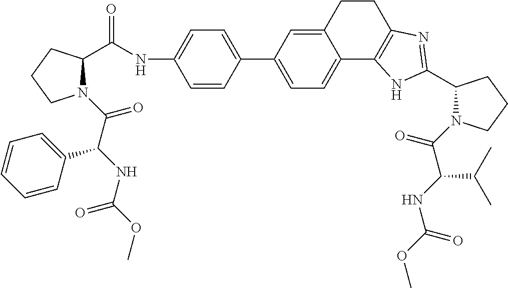 | **** | 776.4 |
| 118 | 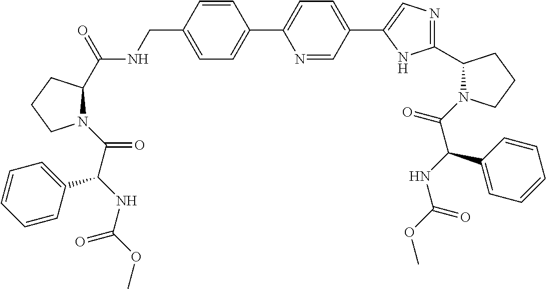 | **** | 799.3 |
| 119 | 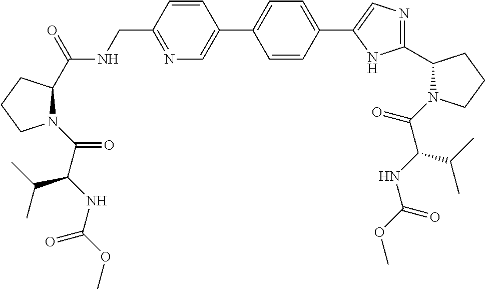 | ** | 731.4 |
| 120 | 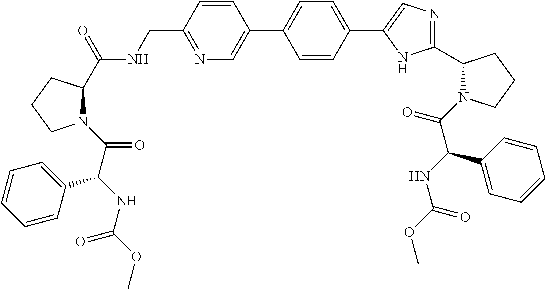 | **** | 799.3 |
| 121 | 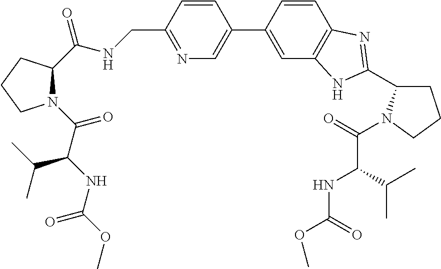 | ** | 705.4 |
| 122 | 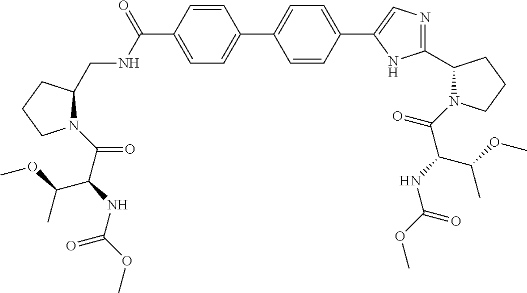 | **** | 762.4 |
| 123 | 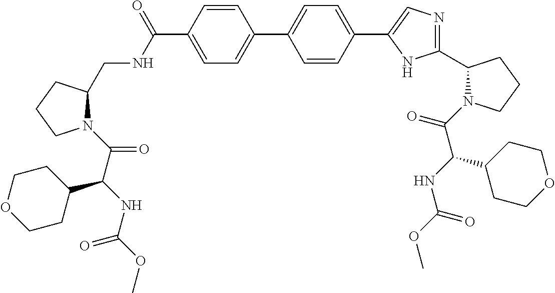 | *** | 814.4 |
| 124 | 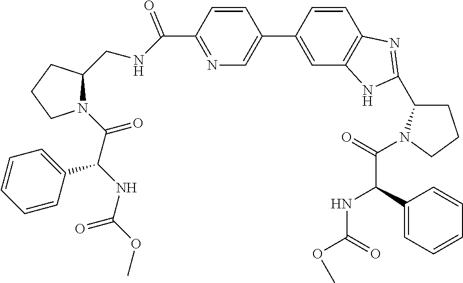 | | 773.3 |

1-55. (canceled)

56. A compound of the formula, I: 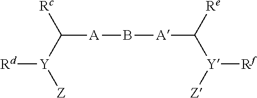
wherein,
A and A′ are independently selected from the group consisting of a single bond, —(CR²)ₙ—C(O)—(CR²)ₚ—, —(CR²)_{μ}—O—(CR²)ₚ—, —(CR²)ₙ—N(R^{N})—(CR²)ₚ—, (CR²)ₙS(O)ₖ—(CR²)ₚ—, —(CR²)ₙS(O)ₖ—N(R^{N})—(CR²)ₚ—, —(CR²)ₙ—C(O)—N(R^{N})—(CR²)ₚ—, —(CR²)ₙ—N(R^{N})—C(O)—N(R^{N})—(CR²)ₚ—, —(CR²)ₙ—C(O)—O—(CR²)ₚ—, —(CR²)ₙ—N(R^{N})—S(O)ₖ—N(R^{N})—(CR²)ₚ— and —(CR²)ₙ—N(R^{N})—C(O)—O—(CR²)ₚ— and a heteroaryl group selected from the group consisting of 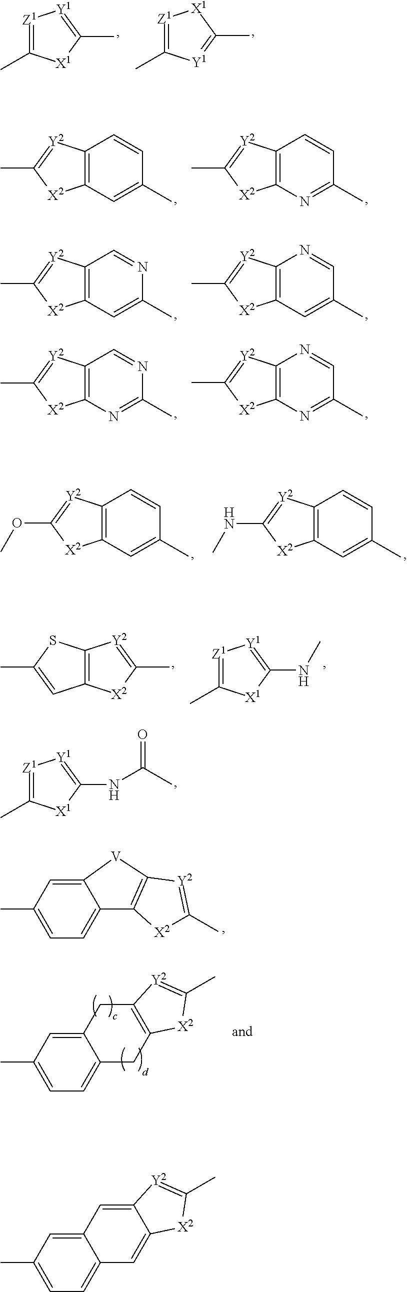
wherein:
X¹ is CH₂, NH, O or S,
Y¹, Y² and Z¹ are each independently CH or N,
X² is NH, O or S,
V is —CH₂—CH₂—, —CH═CH—, —N═CH—, (CH₂)ₐ—N(R^{N})—(CH₂)_{b}— or —(CH₂)—O—(CH₂)_{b}—, wherein a and b are independently 0, 1, 2 or 3 with the proviso that a and b are not both 0, 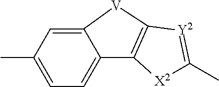
optionally includes 1 or 2 nitrogens as heteroatoms on the phenyl residue,
the carbons of the heteroaryl group are each independently optionally substituted with a substituent selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
the nitrogens, if present, of the heteroaryl group are each independently optionally substituted with a substituent selected from the group consisting of —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
a and b are independently 1, 2 or 3.
c and d are independently 1 or 2,
n and p are independently 0, 1, 2 or 3,
k is 0, 1 or 2,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino,
each R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide,
wherein for each A and A′, B may be attached to either side of A and A′ so that in the example of A or A′ being 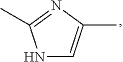
the A-B-A′ can be any of: 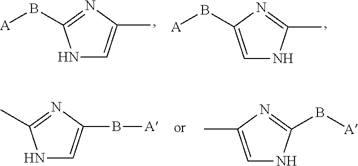
and
wherein only one of A and A′ is a 5-membered heteroaryl ring if B is W—W;
B is W—W or W—X″—W wherein:
each W is an aryl group or a heteroaryl group and X″ is selected from the group consisting of —O—, —S(O)ₖ, —N(R^{N})— and —CR′₂—,
each R′ is independently selected from the group consisting of hydrogen, —OH, —CN, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino and the two R′ are optionally joined to form a 3- to 8-membered ring, and
each W is independently optionally substituted with one or more substituents each independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
R^{e}, R^{d}, R^{e} and R^{f} are each independently selected from the group consisting of: hydrogen, C₁ to C₈ alkyl, C₁ to C₈ heteroalkyl, aralkyl and a 4- to 8-membered ring which may be cycloalkyl, heterocycle, heteroaryl or aryl, wherein,
each hetero atom, if present, is independently N, O or S,
each of R^{c}, R^{d}, R^{e} and R^{f} may optionally be substituted by C₁ to C₈ alkyl, C₁ to C₈ heteroalkyl, aralkyl or a 4- to 8-membered ring which may be cycloalkyl, heterocycle, heteroaryl or aryl and wherein each heteroatom, if present, is independently N, O or S,
R^{c} and R^{d} are optionally joined to form a 4- to 8-membered heterocycle which is optionally fused to another 3- to 6-membered heterocycle or heteroaryl ring, and
R^{e} and R^{f} are optionally joined to form a 4- to 8-membered heterocycle which is optionally fused to another 3- to 6-membered heterocycle or heteroaryl ring;
Y and Y′ are independently carbon or nitrogen; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

57. The compound of claim 56 wherein B is selected from the group consisting of 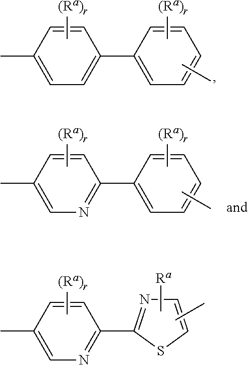
wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

58. The compound of claim 56 wherein B is selected from the group consisting of 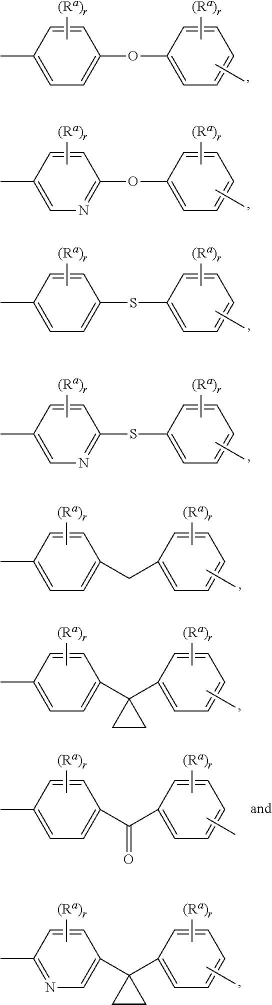
wherein:
each R^{a} is independently selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino; and
each r is independently from 0 to 4.

59. The compound of claim 56 wherein A is —(CR²)ₙ—O—(CR²)ₚ— or —(CR²)ₙ—C(O)—N(R^{N})—(CR²)ₚ—.

60. The compound of claim 56 wherein A′ is selected from the group consisting of

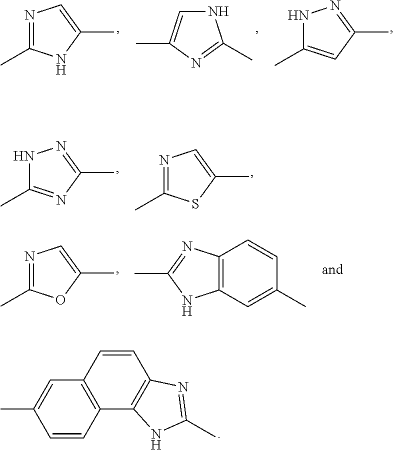

61. The compound of claim 56 wherein R′ and R^{d} are joined and form a heterocyclic fused ring system selected from the group consisting of: 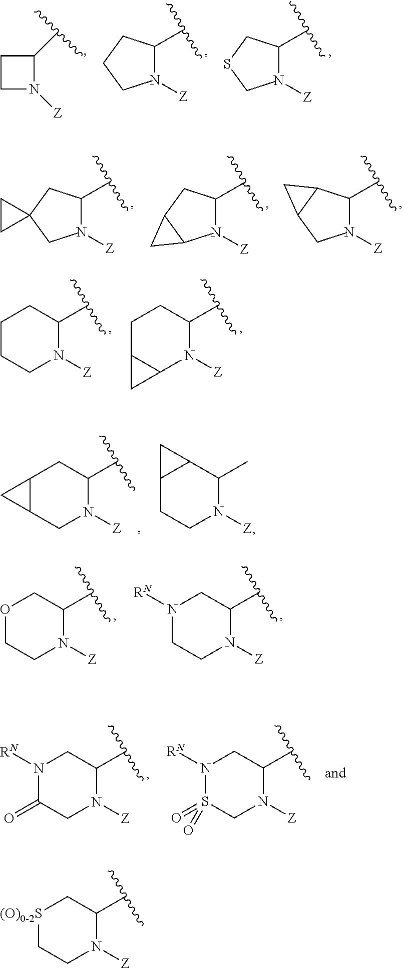
wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide.

62. The compound of claim 56 wherein R^{e} and R^{f} are joined and form a heterocyclic fused ring system selected from the group consisting of: 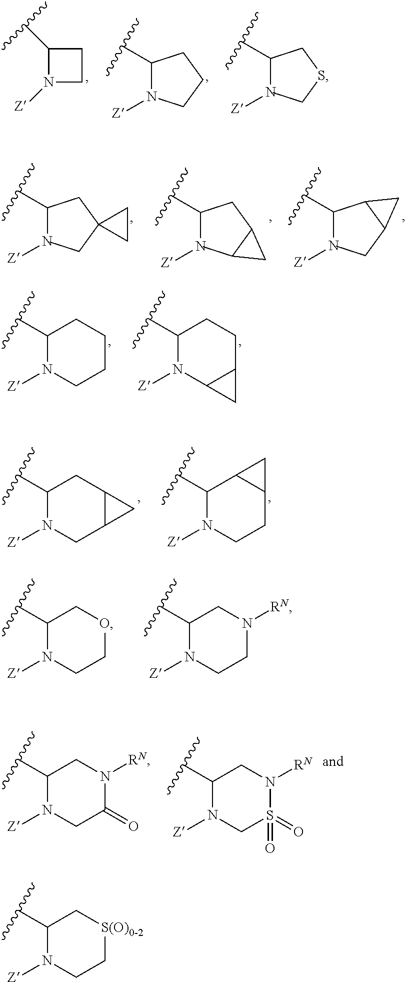
wherein R^{N} is selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide

63. The compound of claim 56 having formula II: 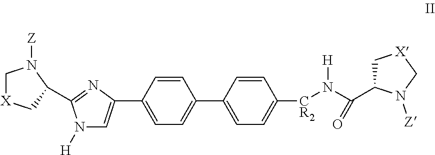
wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

64. The compound of claim 56 having formula III: 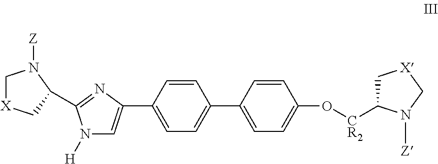
wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

65. The compound of claim 56 having formula IV: 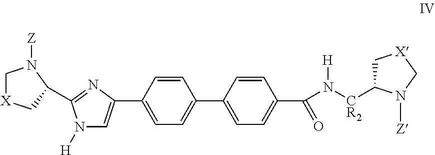
wherein,
each R is independently selected from the group consisting of hydrogen, —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino;
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)₁—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

66. The compound of claim 56 having formula V: 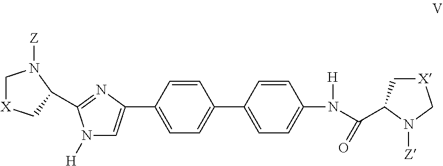
wherein,
X and X′ are each independently selected from the group consisting of a bond, —CH₂—, —CH₂—CH₂—, —CH═CH—, —O—, —S—, S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)₁—R⁸ and —[U—(CR⁴₂)ₜ—NR⁵—(CR⁴₂)ₜ]ᵤ—U—(CR⁴₂)ₜ—O—(CR⁴₂)₁—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

67. The compound of claim 56 having formula VI: 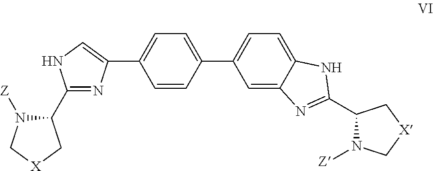
wherein,
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

68. The compound of claim 56 having a formula selected from the group consisting of: 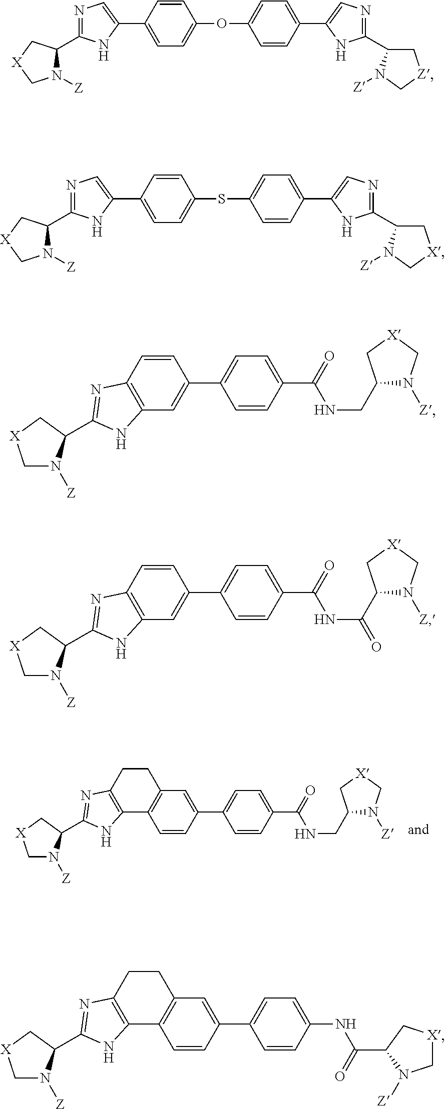
wherein:
X and X′ are each independently selected from the group consisting of a bond, —CH₂—, —CH₂—CH₂—, —CH═CH—, —O—, —S—, —S(O)₁₋₂—, —CH₂O—, —CH₂S—, —CH₂S(O)₁₋₂— and —CH₂N(R¹)—, wherein R¹ is chosen from the group consisting of hydrogen, C₁ to C₈ alkyl, C₁ to C₈ heteroalkyl, cycloalkyl, heterocycle, aryl, heteroaryl, aralkyl, alkanoyl, alkoxycarbonyl, carbamoyl and substituted sulfonyl; and
Z and Z′ are independently selected from the group consisting of hydrogen, C₁ to C₈ alkyl, C₁ to C₈ heteroalkyl, cycloalkyl, heterocycle, aryl, heteroaryl, aralkyl, 1-3 amino acids, [U—(CR⁴₂)ₜ—NR⁵—(CR⁴₂)ₜ]ᵤ—U—(CR⁴₂)ₜ—NR⁷—(CR⁴₂)ₜ—R⁸, —U—(CR⁴₂)₁—R⁸ and —[U—(CR⁴₂)ₜ—NR⁵—(CR⁴₂)ₜ]ᵤ—U—(CR⁴₂)ₜ—O—(CR⁴₂)₁—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3 or 4, and
u is 0, 1 or 2.

69. The compound of claim 56 wherein one or both of Z and Z′ are —C(O)—(CR⁴₂)ₜ—NR⁷—(CR⁴₂)ₜ—R⁸.

70. The compound of claim 56 wherein one or both of Z and Z′ are —C(O)—(CR⁴₂)ₙ—NR⁷—C(O)—O—R⁸¹.

71. The compound of claim 56 wherein one or both of Z and Z′ are —C(O)—(CR⁴₂)ₜ—R⁸.

72. The compound of claim 56 wherein one or both of Z and Z′ are —C(O)—(CR⁴₂)ₜ—O—(CR⁴₂)ₜ—R⁸.

73. The compound of claim 56 having one of the following formulas:

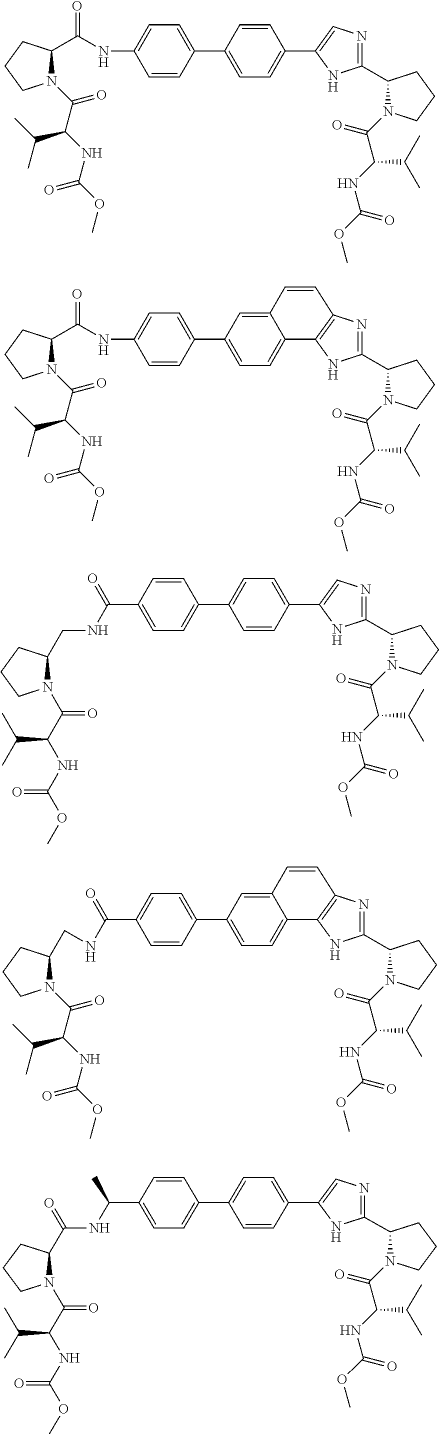

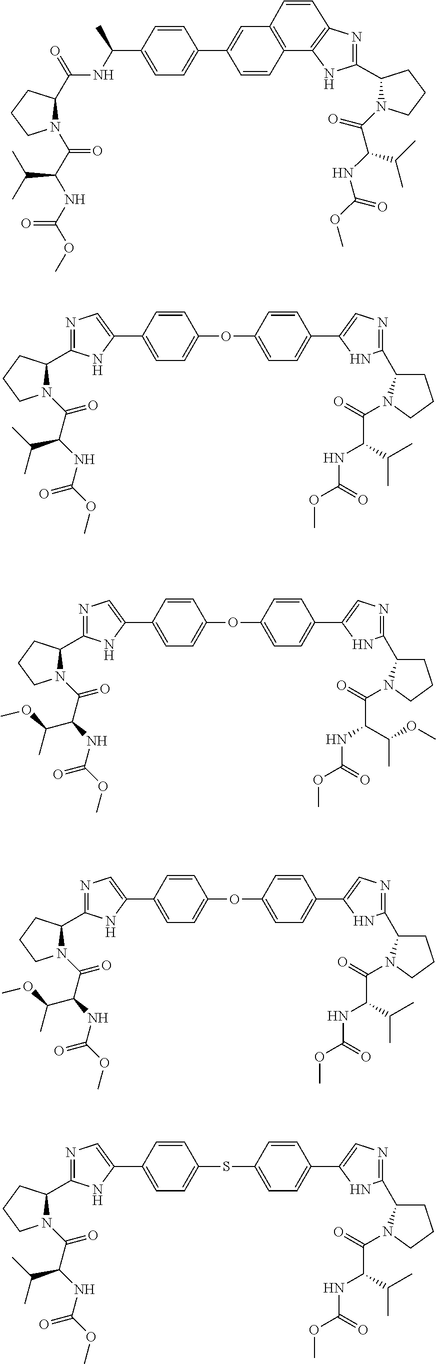

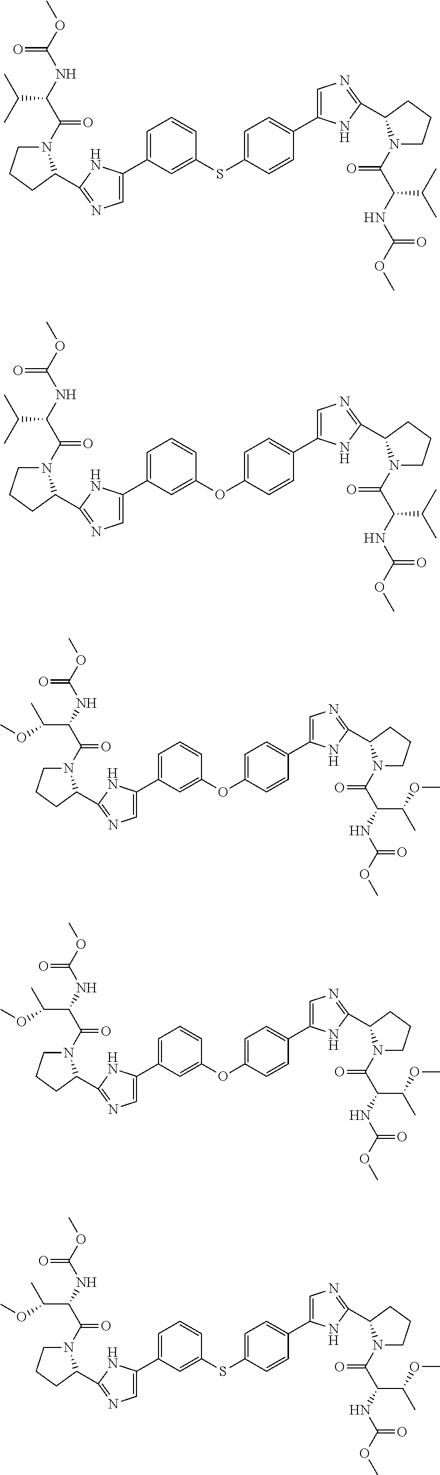

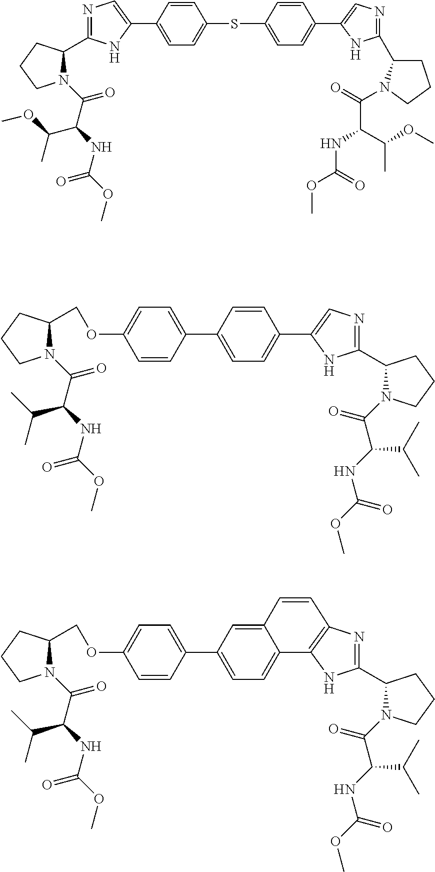

74. A pharmaceutical composition comprising the composition of claim 56.

75. A method of treating hepatitis C comprising administering to a subject in need thereof, a therapeutically effective amount of the compound of claim 56.
